SEQUENCE LISTING (44 sequences)

SEQ ID NO: 1 - protein, 11 aa, Artificial Sequence
  Description of Artificial Sequence Synthetic peptide
RASQDVSTAVA

SEQ ID NO: 2 - protein, 7 aa, Artificial Sequence
  Description of Artificial Sequence Synthetic peptide
SASFLYS

SEQ ID NO: 3 - protein, 9 aa, Artificial Sequence
  Description of Artificial Sequence Synthetic peptide
QQSNRAPAT

SEQ ID NO: 4 - protein, 10 aa, Artificial Sequence
  Description of Artificial Sequence Synthetic peptide
GFTFNGTYIH

SEQ ID NO: 5 - protein, 17 aa, Artificial Sequence
  Description of Artificial Sequence Synthetic peptide
GIYPAGGATYYADSVKG

SEQ ID NO: 6 - protein, 9 aa, Artificial Sequence
  Description of Artificial Sequence Synthetic peptide
WWAWPAFDY

SEQ ID NO: 7 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSNRAPATFGQGTKVEIKR

SEQ ID NO: 8 - protein, 118 aa, Artificial Sequence
  Description of Artificial Sequence Synthetic polypeptide
EVQLVESGGGLVQPGGSLRLSCAASGFTFNGTYIHWVRQAPGKGLEWVGGIYPAGGATYYADSVKGRFTISRDNSKNTLYLQMNSLRAEDTAVYYCAKWWAWPAFDYWGQGTLVTVSS

SEQ ID NO: 9 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSNRAPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 225 aa, Artificial Sequence
  Description of Artificial Sequence Synthetic polypeptide
EVQLVESGGGLVQPGGSLRLSCAASGFTINGTYIHWVRQAPGKGLEWVGGIYPAGGATYYADSVKGRFTISADTSKNTAYLQMNSLRAEDTAVYYCAKWWAWPAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 11 - protein, 214 aa, Artificial Sequence; identical to SEQ ID NO: 9
  Description of Artificial Sequence Synthetic polypeptide

SEQ ID NO: 12 - protein, 224 aa, Artificial Sequence
  Description of Artificial Sequence Synthetic polypeptide
EVQLVESGGGLVQPGGSLRLSCAASGFTINGTYIHWVRQAPGKGLEWVGGIYPAGGATYYADSVKGRFTISADTSKNTAYLQMNSLRAEDTAVYYCAKWAWPAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 13 - protein, 9 aa, Artificial Sequence
  Description of Artificial Sequence Synthetic peptide
AAAHHHHHH

SEQ ID NO: 14 - protein, 650 aa, Homo sapiens
MGRWAWVPSPWPPPGLGPFLLLLLLLLLLPRGFQPQPGGNRTESPEPNATATPAIPTILVTSVTSETPATSAPEAEGPQSGGLPPPPRAVPSSSSPQAQALTEDGRPCRFPFRYGGRMLHACTSEGSAHRKWCATTHNYDRDRAWGYCVEATPPPGGPAALDPCASGPCLGSCSNTQDPQSYHCSCPRAFTGKDCGTEKCFDETRYEYLEGGDRWARVRQGHVEQCECGRTWCEGTRHTACLSSPCLNGGTCHLIVATGTTVCACPPGFAGRLCNIEPDERCFLGNGTGYRGVASTSASGLSCLAWNSDLLYQELHVDSVGAAALLGLGPHAYCRNPDNDERPWCYVKDSALSWEYCRLEACESLTRVQLSPDLLATLPEPASPGRQACGRRHKKRTFLRPRIIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDENVSGYSSSLREALVPLVADHKCSSPEVYGADISPNMLCAGYFDCKSDACQGDSGGPLACEKNGVAYLYGIISWGDGCGRLHKPGVYTRVANYVDWINDRIRPPRRLVAPS

SEQ ID NO: 15 - protein, 283 aa, Homo sapiens
VQLSPDLLATLPEPASPGRQACGRRHKKRTFLRPRIIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDENVSGYSSSLREALVPLVADHKCSSPEVYGADISPNMLCAGYFDCKSDACQGDSGGPLACEKNGVAYLYGIISWGDGCGRLHKPGVYTRVANYVDWINDRIRPPRRLVAPS

SEQ ID NO: 16 - protein, 239 aa, Homo sapiens
IIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDENVSGYSSSLREALVPLVADHKCSSPEVYGADISPNMLCAGYFDCKSDACQGDSGGPLACEKNGVAYLYGIISWGDGCGRLHKPGVYTRVANYVDWINDRIR

SEQ ID NO: 17 - protein, 108 aa, Artificial Sequence; identical to SEQ ID NO: 7
  Description of Artificial Sequence Synthetic polypeptide

SEQ ID NO: 18 - protein, 108 aa, Artificial Sequence; identical to SEQ ID NO: 7
  Description of Artificial Sequence Synthetic polypeptide

SEQ ID NO: 19 - protein, 118 aa, Artificial Sequence; identical to SEQ ID NO: 8
  Description of Artificial Sequence Synthetic polypeptide

SEQ ID NO: 20 - protein, 117 aa, Artificial Sequence
  Description of Artificial Sequence Synthetic polypeptide
EVQLVESGGGLVQPGGSLRLSCAASGFTFNGTYIHWVRQAPGKGLEWVGGIYPAGGATYYADSVKGRFTISRDNSKNTLYLQMNSLRAEDTAVYYCAKWAWPAFDYWGQGTLVTVSS

SEQ ID NO: 21 - protein, 4 aa, Artificial Sequence
  Description of Artificial Sequence Synthetic peptide
KQLR

SEQ ID NO: 22 - protein, 4 aa, Artificial Sequence
  Description of Artificial Sequence Synthetic peptide
RQLR

SEQ ID NO: 23 - protein, 9 aa, Artificial Sequence
  Description of Artificial Sequence Synthetic peptide
QQSYTTPPT

SEQ ID NO: 24 - protein, 6 aa, Artificial Sequence
  Description of Artificial Sequence Synthetic peptide
NGTYIH

SEQ ID NO: 25 - protein, 18 aa, Artificial Sequence
  Description of Artificial Sequence Synthetic peptide
GGIYPAGGATYYADSVKG

SEQ ID NO: 26 - protein, 10 aa, Artificial Sequence
  Description of Artificial Sequence Synthetic peptide
KWWAWPAFDY

SEQ ID NO: 27 - protein, 9 aa, Artificial Sequence
  Description of Artificial Sequence Synthetic peptide
KWAWPAFDY

SEQ ID NO: 28 - protein, 248 aa, Homo sapiens
IIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDENVSGYSSSLREALVPLVADHKCSSPEVYGADISPNMLCAGYFDCKSDACQGDSGGPLACEKNGVAYLYGIISWGDGCGRLHKPGVYTRVANYVDWINDRIRPPRRLVAPS

SEQ ID NO: 29 - protein, 223 aa, Homo sapiens
IVGGYTCGANTVPYQVSLNSGYHFCGGSLINSQWVVSAAHCYKSGIQVRLGEDNINVVEGNEQFISASKSIVHPSYNSNTLNNDIMLIKLKSAASLNSRVASISLPTSCASAGTQCLISGWGNTKSSGTSYPDVLKCLKAPILSDSSCKSAYPGQITSNMFCAGYLEGGKDSCQGDSGGPVVCSGKLQGIVSWGSGCAQKNKPGVYTKVCNYVSWIKQTIASN

SEQ ID NO: 30 - protein, 241 aa, Homo sapiens [sequence omitted]

SEQ ID NO: 31 - protein, 255 aa, Homo sapiens
IVGGRDTSLGRWPWQVSLRYDGAHLCGGSLLSGDWVLTAAHCFPERNRVLSRWRVFAGAVAQASPHGLQLGVQAVVYHGGYLPFRDPNSEENSNDIALVHLSSPLPLTEYIQPVCLPAAGQALVDGKICTVTGWGNTQYYGQQAGVLQEARVPIISNDVCNGADFYGNQIKPKMFCAGYPEGGIDACQGDSGGPFVCEDSISRTPRWRLCGIVSWGTGCALAQKPGVYTKVSDFREWIFQAIKTHSEASGMVTQL

SEQ ID NO: 32 - protein, 253 aa, Homo sapiens
IIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 33 - protein, 254 aa, Homo sapiens [sequence omitted]

SEQ ID NO: 34 - protein, 259 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 35 - protein, 243 aa, Homo sapiens
VVGGLVALRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYVQPVCLPSGAARPSETTLCQVAGWGHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS

SEQ ID NO: 36 - protein, 252 aa, Homo sapiens
IKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 37 - protein, 51 aa, Homo sapiens
CLASNKVGRCRGSFPRWYYDPTEQICKSFVYGGCLGNKNNYLREEECILAC

SEQ ID NO: 38 - protein, 51 aa, Homo sapiens
CLVSKVVGRCRASMPRWWYNVTDGSCQLFVYGGCDGNSNNYLTKEECLKKC

SEQ ID NO: 39 - protein, 51 aa, Homo sapiens
CVDLPDTGLCKESIPRWYYNPFSEHCARFTYGGCYGNKNNFEEEQQCLESC

SEQ ID NO: 40 - protein, 51 aa, Homo sapiens
CTANAVTGPCRASFPRWYFDVERNSCNNFIYGGCRGNKNSYRSEEACMLRC

SEQ ID NO: 41 - protein, 51 aa, Homo sapiens
CAFKADDGPCKAIMKRFFFNIFTRQCEEFIYGGCEGNQNRFESLEECKKMC

SEQ ID NO: 42 - protein, 51 aa, Homo sapiens
CFLEEDPGICRGYITRYFYNNQTKQCERFKYGGCLGNMNNFETLEECKNIC

SEQ ID NO: 43 - protein, 51 aa, Homo sapiens
CLTPADRGLCRANENRFYYNSVIGKCRPFKYSGCGGNENNFTSKQECLRAC

SEQ ID NO: 44 - protein, 51 aa, Homo sapiens
CLEPPYTGPCKARIIRYFYNAKAGLCQTFVYGGCRAKRNNFKSAEDCMRTC

(19) United States
(12) Patent Application Publication
(10) Pub. No.: US 2011/0206704 A1
(43) Pub. Date: Aug. 25, 2011

(54) Methods and compositions for modulating hepatocyte growth factor activator

(75) Inventors: Rajkumar Ganesan, North Potomac, MD (US); Daniel Kirchhofer, Los Altos, CA (US)

(73) Assignee: Genentech, Inc., South San Francisco, CA (US)

(21) Appl. No.: 12/925,379

(22) Filed: Oct. 18, 2010

Related U.S. Application Data
(60) Provisional application No. 61/253,014, filed on Oct. 19, 2009.
(60) Provisional application No. 61/281,981, filed on Nov. 25, 2009.

(51) Int. Cl. A61K 39/395; G01N 33/68; C12N 9/96; C12N 9/64; C07K 5/11; C07K 16/40; A61P 35/00; A61P 35/04; G06T 15/00

(52) U.S. Cl. 424/178.1; 435/7.1; 435/188; 435/226; 530/330; 530/389.3; 345/419

(57) ABSTRACT

The present invention relates generally to the fields of molecular biology and growth factor regulation. More specifically, the invention concerns modulators of hepatocyte growth factor activator function, and the identification and uses of said modulators.

### REFERENCE TO TABLES SUBMITTED ON A COMPACT DISC

This application is accompanied by Tables submitted on a compact disc (in duplicate copies) which contains the file titled “Table 7.txt”, created on 23 Nov. 2009, that is 480,704 bytes in size, the file titled “Table 8.txt”, created on 23 Nov. 2009, that is 723,492 bytes in size, and the file titled “Table 9.txt”, created on 23 Nov. 2009, that is 446,483 bytes in size, the contents of which are incorporated in their entirety herein by reference. The table submission contains Tables 7, 8 and 9.

LENGTHY TABLES
The patent application contains a lengthy table section. A copy of the table is available in electronic form from the USPTO web site (http://seqdata.uspto.gov/?pageRequest=docDetail&DocID=US20110206704A1). An electronic copy of the table will also be available from the USPTO upon request and payment of the fee set forth in 37 CFR 1.19(b)(3).

### SEQUENCE LISTING

The instant application contains a Sequence Listing which has been submitted in ASCII format via EFS-Web and is hereby incorporated by reference in its entirety. Said ASCII copy, created on Oct. 14, 2010, is named P4324R1U.txt and is 52,067 bytes in size.

### TECHNICAL FIELD

The present invention relates generally to the fields of molecular biology and growth factor regulation. More specifically, the invention concerns modulators of hepatocyte growth factor activator function, and the identification and uses of said modulators.

### BACKGROUND

Hepatocyte growth factor activator (HGFA) is a plasma trypsin-like serine protease secreted mainly by the liver that regulates the mitogenic, motogenic, and morphogenic activities of hepatocyte growth factor (HGF, also known as scatter factor (SF)) (Shimomura et al., Cytotech., 8:219-229 (1992)). HGF is implicated in embryonic development, tissue regeneration and invasive tumor growth. This activity requires proteolytic processing of HGF into a two-chain, disulfide-linked α,β-heterodimeric form. HGFA is among the most potent activators of HGF identified so far. (Shimomura et al., Eur. J. Biochem. 229 (1995)). HGFA expression has been reported in normal gastrointestinal renal tissues, and in the central nervous system, as well as in pancreatic, hepatocellular, colorectal, prostatic, and lung cancer cells. (Itoh et al., Biochim. Biophys. Acta, 1491:295-302 (2000); van Adelsberg et al., J. Biol. Chem., 276:15099-15106 (2001); Hayashi et al., Brain Res., 799:311-316 (1998); Moriyama et al., FEBS Lett., 372:78-82 (1995); Parr et al., Int. J. Oncol., 19:857-863 (2001); Kataoka et al., Cancer Res., 60:6148-6159 (2000); Nagata et al., Biochem. Biophys. Res. Comm., 289:205-211 (2001)). Recently, HGFA secretion from multiple myeloma cells has been linked to the potent para- and/or autocrine effect of HGF. (Tjin et al., Blood, 104:2172-2175 (2004)).

HGFA is secreted as a 96 kDa zymogen (proHGFA) with a domain structure like that of coagulation factor XII (FXIIa), comprising 6 domains. Those domains include an N-terminal fibronectin type II domain, an epidermal growth factor (EGF)-like domain, a fibronectin type 1 domain, another EGF-like domain, a kringle domain, and a C-terminal trypsin homology serine protease domain. (Miyazawa, et al., J. Biol. Chem., 268:10024-10028 (1993)). Cleavage at a kallikrein-sensitive site between residues Arg372 and Val373 can produce a short 34 kDa form that lacks the first 5 domains. Both the 96 kDa and 34 kDa forms of proHGFA can be cleaved between residues Arg407 and Ile408 into active HGFA by thrombin. (Shimomura et al., J. Biol. Chem., 268, 22927-22932 (1993)). Thrombin is the ultimate effector of pro-coagulant stimuli and generation of active HGFA would be consistent with the activity of HGF in wound repair. (Bussolino et al., J. Cell Biol., 119:625-641 (1992)).

Among factors influencing HGF/Met signaling are the activation of proHGFA and subsequent inhibition of HGFA. The identified physiological inhibitors of HGFA are the splice variants HAI-1 and HAI-1B (hepatocyte growth factor activator inhibitor-1), and HAI-2 (also known as placental bikunin). (Shimomura et al., J. Biol. Chem., 272:6370-6376 (1997); Kawaguchi et al., J. Biol. Chem., 272:27558-27564 (1997); Kirchhofer et al., J. Biol. Chem. 278:36341-36349 (2003)). HGFA has restricted substrate specificity (Kataoka et al., Cancer metastasis reviews 22, 223-239 (2005); Miyazawa et al., J Biol Chem 268, 10024-10028 (1993)): only two macromolecular substrates, pro-hepatocyte growth factor (pro-HGF) (Shinomura et al., Eur J Biochem 229, 257-261 (1995) and pro-macrophage stimulating protein (pro-MSP) (Kawaguchi et al, Febs J 276(13)3481-3490 (2009), are known to be processed by HGFA, exemplifying the enzyme's highly restricted substrate specificity. HGFA is inhibited by the Kunitz-type inhibitor HGFA inhibitor-1, which utilizes the N-terminal Kunitz domain-1 (KD1) to inhibit HGFA by a canonical inhibition mechanism (Shia et al., J Mol Biol 346, 1335-13492005). HGFA effects tissue regeneration and promotes cancer growth via pro-HGF processing and ensuing activation of the HGF/Met signaling pathway (Parr and Jiang, Int'l J of Oncol 19, 857-863 (2001)).

Allosteric regulation of an enzyme, by definition, involves an altered catalytic activity originating from a remote effector interaction site. In fact, all dynamic proteins (monomeric and multimeric) seem to have a potential for allosterism (Gunasekaran et al., Proteins 57, 433-443 (2004)). Elucidation of allosteric modulation and its pathways of communication have received considerable attention (Swain and Gierasch, Curr Op in Structural Biol 16, 102-108 (2006); Yu and Koshland, PNAS 98, 9517-9520 (2001)). A classic example of allostery is observed in hemoglobin (Perutz, Nature 228, 726-739 (1970)), which offered the first mechanistic insights on allosteric regulation. Several X-ray crystallographic studies emerged thereafter describing the conformational changes during allosteric regulation (Changeux and Edelstein, Science 308, 1424-1428 (2005); Di Cera, J Biol Chem 281, 1305-1308 (2006); Pellicena and Kuriyan, Nature 228, 726-739 (2006); Xu et al., Nature 388, 741-750 (1997)). Allostery is also a quite common and powerful mechanism to regulate the catalytic activity of proteases (Hauske et al., Chembiochem 9, 2920-2928 (2008); Turk, Nature Reviews 5, 785-799 (2006)). Unlike active sites, distally located allosteric sites are usually less conserved and can be exploited to achieve specificity (Hauske et al., supra). Allosteric anti-protease protein-based agents have great therapeutic potential, since they are potent and highly specific and are safeguarded from any inadvertent processing by their target protease. Examples of allosteric regulators in the serine protease family (Clan PA, Family S1 in MEROPS nomenclature (Rawlings et al., Nucleic Acids Res 36, D320-325 (2008))) are the accessory PDZ domains in the HtrA protease family (Sohn et al., Cell 131, 572-583 (2007)), calcium for many coagulation factors (Bjelke et al., J Biol Chem 283, 25863-25870 (2008)), sodium for thrombin (Huntington, Biological chemistry 389, 1025-1035 (2008); Wells and Di Cera, Biochem 31, 11721-11730 (1992)), cofactors such as tissue factor for coagulation factor VIIa (Eigenbrot and Kirchhofer, Trends in Cardiovascular Med 12, 19-26 (2002)) and N-terminal peptide insertion into the “activation pocket” (Friedrich et al., Nature 425, 535-539 (2003); Huber and Bode, Acc Chem Res 11, 114-122 (1978)).

Proteases have been implicated in many human pathological processes (Barrett et al., (1998). Handbook of Proteolytic Enzymes. San Diego: Academic Press (1998); Egeblad and Werb, Nature Rev Cancer 2, 161-174 (2002); Hooper, Proteases in Biology and Medicine. In Essays in Biochemistry, London: Portland Press (2002); Luttun et al., Curr Atheroscler. Rep 2, 407-416 (2000)). Therefore, regulation of proteolytic activity by allosteric inhibitors might represent a promising alternative approach to active site inhibitors (Peterson and Golemis, J Cell Biochem 93, 68-73 (2004)), which often suffer from inadequate specificity, since active site topologies are generally conserved among members of the same family (Hedstrom, Chem Revs 102, 4501-4524 (2002)). Unlike active sites, distally located allosteric sites are usually less conserved and can be exploited to achieve specificity (Hauske et al., supra). Excellent examples of specific and potent allosteric inhibitors have been described for coagulation factor VIIa and caspases (Hardy et al., PNAS101, 12461-12466 (2004); Hardy and Wells, Curr Op Structural Biol 14, 706-715 (2009)).

Since activation of pro-HGF requires cleavage by a convertase such as HGFA, modulation of HGFA function and/or its interaction with its substrate could prove to be an efficacious therapeutic approach. In this regard, there is a clear need to identify clinically relevant agents capable of modulating activity of and/or specifically interacting with HGFA. The invention fulfills this need and provides other benefits.

All publications, patents, and patent documents are incorporated by reference herein, as though individually incorporated by reference.

### SUMMARY OF THE INVENTION

The invention provides methods, composition, kits and articles of manufacture for modulating hepatocyte growth factor activator (HGFA) function, thereby modulating physiological effects of HGFA activity. Described herein is the identification of a hydrophobic binding pocket, termed an allosteric binding site, of HGFA that favorably associates with an allosteric inhibitor, whereby HGFA enzymatic activity is altered (inhibited) from a location that is different from the catalytic site. An exemplary allosteric inhibitor compound is described. Structural and kinetic studies described herein provide a detailed view of how an allosteric inhibitor inhibits HGFA catalysis. The data described herein provide a basis for design of HGFA antagonists capable of inhibiting wild type HGFA activation. These antagonists can be used as advantageous therapeutic agents for treating pathological conditions wherein reduced HGF/c-met biological activity is desirable. Methods and compositions of the invention are based generally on these findings, which are described in greater detail below. Thus, the invention provides methods, compositions, kits and articles of manufacture for identifying and using substances that are capable of modulating the HGF/c-met pathway through modulation of HGFA activation, and for modulation of biological/physiological activities associated with HGF/c-met signaling.

In one aspect, the amino acid residues that form a binding site for an allosteric inhibitor of HGFA are identified and are useful, for example, in methods to model the structure of an HGFA allosteric binding site and methods to identify agents that can bind or fit into the binding site. This use includes the rational design of modulators of HGFA activity. For example, these modulators include ligands that interact with HGFA and modulate HGFA activities, such as HGFA activation of pro-HGF, HGFA catalytic activity, cell migration, and Met phosphorylation and signaling.

In one aspect, the invention provides methods of identifying (screening) a candidate inhibitor substance that inhibits (e.g., allosterically inhibits) HGFA activation of HGFA substrate, said method comprising: (a) contacting a candidate substance with a first sample comprising HGFA and a HGFA substrate, (b) comparing amount of HGFA substrate activation in the sample with amount of HGFA substrate activation in a reference sample comprising similar amounts of HGFA and HGFA substrate as the first sample but that has not been contacted with said candidate substance, whereby a decrease in amount of HGFA substrate activation in the first sample compared to the reference sample indicates that the candidate substance is capable of inhibiting HGFA activation of HGFA substrate; and (c) detecting binding, if any, of the candidate substance to HGFA allosteric binding site.

In one aspect, the invention provides methods of identifying (screening) a candidate inhibitor substance that allosterically inhibits HGFA activation of HGFA substrate, said method comprising: detecting binding, if any, of the candidate substance to a HGFA allosteric binding site, wherein the candidate substance is identified by a method comprising comparing amount of HGFA substrate activation in a sample with amount of HGFA substrate activation in a reference sample comprising similar amounts of HGFA and HGFA substrate as the first sample but that has not been contacted with said candidate substance, whereby a decrease in amount of HGFA substrate activation in the first sample compared to the reference sample indicates that the candidate substance is capable of inhibiting HGFA activation of HGFA substrate (eg, single chain HGF (pro-HGF)), wherein the sample comprises HGFA, the candidate substance and the HGFA substrate.

In one aspect, the invention provides methods of screening for (identifying) a candidate inhibitory substance that inhibits HGFA activation, the method comprising screening for a substance that binds (in some embodiments, specifically binds) HGFA allosteric binding site and inhibits HGFA activity.

In one aspect, the invention provides methods of screening for (identifying) a candidate inhibitory substance that inhibits (e.g., allosterically blocks) HGFA activation, the method comprising screening for a substance that binds HGFA in the absence of a compound that blocks HGFA active (catalytic) site, but does not bind HGFA in the presence of the compound that blocks HGFA active site. In some embodiments, the compound that blocks HGFA active site is Ac-KQLR-chloromethyl ketone (“KQLR” disclosed as SEQ ID NO: 21). In some embodiments, the compound that blocks HGFA active site is KD1.

In one aspect, the invention provides methods of screening for (identifying) a candidate inhibitory substance that inhibits (e.g., allosterically blocks) HGFA activation, the method comprising screening for a substance that competes for binding to HGFA with HGFA active site blocker KD1 or Ac-KQLR-chloromethyl ketone (“KQLR” disclosed as SEQ ID NO: 21), but does not compete for binding to HGFA with benzamidine.

In one aspect, the invention provides methods of screening for (identifying) a candidate inhibitory substance that inhibits (e.g., allosterically blocks) HGFA activation, the method comprising screening for a substance that competes for binding to HGFA with an allosteric inhibitor of HGFA. In one embodiment, the allosteric HGFA inhibitor is antibody Fab40. In some embodiments, the methods further comprise detecting binding of the substance to HGFA allosteric binding site. In some embodiments, the methods further comprise determining whether the substance competes for binding to HGFA with HGFA active site blockers (eg, Ac-KQLR-chloromethyl ketone (“KQLR” disclosed as SEQ ID NO: 21)).

In some embodiments of methods of screening (identifying) of the invention, the methods comprise determining binding affinity of a candidate substance with respect to a target antigen which comprises, consists or consists essentially of a portion or all of HGFA. Such target antigens can include any polypeptide that comprises, consists or consists essentially of an HGFA allosteric binding site, or portions thereof. In some embodiments, the polypeptides comprise, consist or consist essentially of an HGFA allosteric binding site amino acid sequence fused to a heterologous polypeptide sequence.

In another aspect, the invention provides a method of screening for an HGFA antagonist which blocks HGFA activity (such as allosterically blocks HGFA activity), said method comprising selecting for a substance that binds to at least one, two, three, four, or any number up to all of residues 446, 449, 450, 452, 453, 455, 480, 481, 482, 483, 484, 485, 486, 487, 488, 489, 490, 491, 496, 499, 501, 578, 579, 580, 636, 637, 640, 643, 644 of HGFA. In some embodiments, said method comprises selecting for a substance that binds to at least one, two, three, four, or any number up to all of residues 449, 450, 452, 482, 484, 485, 486, 487, 488, 489, 490, 491 of HGFA. In some embodiments, said method comprises selecting for a substance that binds to at least one, two, three, four, or any number up to all of residues 446, 482, 484, 490, 499, 501 of HGFA.

In one embodiment, the substance binds to a sequence comprising one or more of (a) residues 449-455 of HGFA, (b) residues 480-491 of HGFA, (c) residues 579-581 of HGFA, and/or (d) residues 637-645 of HGFA. In one embodiment, the substance binds to a sequence comprising one or more of (a) residues 451-454 of HGFA and/or (b) residues 490-493 of HGFA.

In some embodiments, HGFA substrate is a synthetic HGFA substrate (e.g., Chromogenix S-2266 (H-D-Val-Leu-Arg-pNA). In some embodiments HGFA substrate is pr-HGF (single chain HGF). In some embodiments, the pro-HGF substrate is a polypeptide comprising HGF or fragment thereof comprising a wild type form of the R494-V495 peptide linkage. In some embodiments, the pro-HGF substrate comprises a cleavage site of human HGF that fits the consensus cleavage site of proteases wherein the cleavage site comprises basic residue at position P₁ and two hydrophobic amino acid residues in positions P₁′ and P₂′. In some embodiments, HGFA in the sample is in an effective amount for activating HGFA substrate.

In some embodiments of methods of screening (identifying), the methods further comprise determining a crystal structure of a candidate substance bound to HGFA (e.g., bound to HGFA allosteric binding site).

As would be evident to one skilled in the art, screening assays consistent with those described above can also comprise a first step of screening based on a functional readout to obtain a first set of candidate modulatory substance, followed by a second step of screening based on ability of the first set of candidate modulatory substance to allosterically inhibit HGFA activation (e.g., by binding HGFA allosteric binding site, whereby HGFA activity, such as HGFA enzymatic activity is reduced or inhibited). In some embodiments, HGFA enzymatic activity comprises cleavage of polypeptide substrate of HGFA. In one embodiment, the polypeptide substrate of HGFA is pro-HGF. A functional readout can be any that would be evident to one skilled in the art, based on a knowledge of biological activities associated with the HGF/c-met signaling pathway. These biological activities include but are not limited to C-met phosphorylation, phosphorylation of cellular molecules that are substrates of C-met kinase, cellular growth (proliferation, survival, etc.), angiogenesis, cell migration, cell morphogenesis, etc.

In one aspect, the invention provides HGFA antagonists that disrupt the HGF/c-met signaling pathway. For example, the invention provides a molecule that binds (in some embodiments, specifically binds) HGFA and inhibits (in some embodiments, allosterically inhibits) HGFA activity.

In some embodiments, the HGFA antagonist substance binds to at least one, two, three, four, or any number up to all of residues 446, 449, 450, 452, 453, 455, 480, 481, 482, 483, 484, 485, 486, 487, 488, 489, 490, 491, 496, 499, 501, 578, 579, 580, 636, 637, 640, 643, 644 of HGFA. In some embodiments, the substance binds to at least one, two, three, four, or any number up to all of residues 449, 450, 452, 482, 484, 485, 486, 487, 488, 489, 490, 491 of HGFA. In some embodiments, the substance binds to at least one, two, three, four, or any number up to all of residues 446, 482, 484, 490, 499, 501 of HGFA. In one embodiment, the substance binds to a sequence comprising one or more of (a) residues 449-455 of HGFA, (b) residues 480-491 of HGFA, (c) residues 579-581 of HGFA, and/or (d) residues 637-645 of HGFA. In one embodiment, the substance binds to a sequence comprising one or more of (a) residues 451-454 of HGFA and/or (b) residues 490-493 of HGFA.

In some embodiments, the substance or molecule is or comprises a small molecule, peptide, antibody, antibody fragment, aptamer, or a combination thereof.

In some embodiments, the substance or molecule comprises an anti-HGFA antibody comprising: at least one, two, three, four, five, and/or six hypervariable region (HVR) sequences selected from the group consisting of: (a) HVR-L1 comprising sequence RASQDVSTAVA (SEQ ID NO:1); (b) HVR-L2 comprising sequence SASFLYS (SEQ ID NO:2); (c) HVR-L3 comprising sequence QQSNRAPAT (SEQ ID NO:3); (d) HVR-H1 comprising sequence GFTFNGTYIH (SEQ ID NO:4); (e) HVR-H2 comprising sequence GIYPAGGATYYADSVKG (SEQ ID NO:5); and (f) HVR-H3 comprising sequence WWAWPAFDY (SEQ ID NO:6). In some embodiments, the substance or molecule comprises an anti-HGFA antibody comprising a light chain variable domain having the sequence:

DIQMTQSPSSLSASVGDRVTITCRASQDVSTAVAWYQQKPGKAPKLLIYS ASFLYSGVPSRFSGSGSGTDFTLTISSLQPEDFATYYCQQSNRAPATFGQGTKVEI KR (SEQ ID NO:7); and further comprises a heavy chain variable domain. In some embodiments, the substance or molecule comprises an anti-HGFA antibody comprising a heavy chain variable domain having the sequence: EVQLVESGGGLVQPGGSLRLSCAASGFTFNGTYIHWVRQAPGKGLEWVGGIYPA GGATYYADSVKGRFTISRDNSKNTLYLQMNSLRAEDTAVYYCAKWWAWPAFD YWGQGTLVTVSS (SEQ ID NO:8); and further comprises a light chain variable domain. In some embodiments, the substance or molecule comprises an anti-HGFA antibody comprising a light chain variable domain having the sequence:

DIQMTQSPSSLSASVGDRVTITCRASQDVSTAVAWYQQKPGKAPKLLIYS ASFLYSGVPSRFSGSGSGTDFTLTISSLQPEDFATYYCQQSNRAPATFGQGTKVEI KR (SEQ ID NO:7); and a heavy chain variable domain having the sequence: EVQLVESGGGLVQPGGSLRLSCAASGFTFNGTYIHWVRQAPGKGLEWVGGIYPA GGATYYADSVKGRFTISRDNSKNTLYLQMNSLRAEDTAVYYCAKWWAWPAFD YWGQGTLVTVSS (SEQ ID NO:8).

In some embodiments, binding of a substance or molecule of the invention to HGFA inhibits HGFA activation of HGFA substrate, eg, pro-HGF. In some embodiments, binding or the substance or molecule of the invention to HGFA competitively in inhibits HGFA activation of HGFA substrate. In one embodiment, the substance binds HGFA in the absence of a compound that blocks HGFA active (catalytic) site, but does not bind HGFA in the presence of the compound that blocks HGFA active site. In some embodiments, the compound that blocks HGFA active site is Ac-KQLR-chloromethyl ketone (“KQLR” disclosed as SEQ ID NO: 21). In some embodiments, the compound that blocks HGFA active site is KD1. In some embodiments, the substance competes for binding to HGFA with HGFA active site blocker KD1 or Ac-KQLR-chloromethyl ketone (“KQLR” disclosed as SEQ ID NO: 21), but does not compete for binding to HGFA with benzamidine.

In some embodiments, binding of said substance or molecule to HGFA (such as HGFA allosteric binding site) inhibits cell growth (such as cell proliferation, survival, angiogenesis, morphogenesis, migration) induced by HGF. In some embodiments, binding of said substance or molecule to HGFA inhibits c-met receptor activation. In some embodiments, binding of a substance or molecule of the invention to HGFA inhibits HGFA substrate binding to HGFA. In some embodiments, binding of a substance or molecule of the invention to HGFA does not inhibit HGFA does not inhibit substrate binding to HGFA. In some embodiments, binding of a substance or molecule of the invention to HGFA inhibits HGFA activity, such as HGFA enzymatic activity. In some embodiments, HGFA enzymatic activity comprises cleavage of polypeptide substrate of HGFA. In one embodiment, the polypeptide substrate of HGFA is pro-HGF.

In some embodiments, a substance or molecule of the invention is obtained by a screening or identification method of the invention as described herein.

In one aspect, the invention provides use of a modulator molecule of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a nucleic acid of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of an expression vector of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a host cell of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of an article of manufacture of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a kit of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder

In one aspect, the invention provides a method of inhibiting c-met activated cell proliferation, said method comprising contacting a cell or tissue with an effective amount of a modulator molecule of the invention, whereby cell proliferation associated with c-met activation is inhibited.

In one aspect, the invention provides a method of treating a pathological condition associated with dysregulation of c-met activation in a subject, said method comprising administering to the subject an effective amount of a modulator molecule of the invention, whereby said condition is treated.

In one aspect, the invention provides a method of inhibiting the growth of a cell that expresses c-met or hepatocyte growth factor, or both, said method comprising contacting said cell with a modulator molecule of the invention thereby causing an inhibition of growth of said cell. In one embodiment, the cell is contacted by HGF expressed by a different cell (e.g., through a paracrine effect).

In one aspect, the invention provides a method of therapeutically treating a mammal having a cancerous tumor comprising a cell that expresses c-met or hepatocyte growth factor, or both, said method comprising administering to said mammal an effective amount of an a modulator molecule of the invention, thereby effectively treating said mammal. In one embodiment, the cell is contacted by HGF expressed by a different cell (e.g., through a paracrine effect).

In one aspect, the invention provides a method for treating or preventing a cell proliferative disorder associated with increased expression or activity of HGFA, said method comprising administering to a subject in need of such treatment an effective amount of an a modulator molecule of the invention, thereby effectively treating or preventing said cell proliferative disorder. In one embodiment, said proliferative disorder is cancer.

In one aspect, the invention provides a method for treating or preventing a cell proliferative disorder associated with increased expression or activity of c-met or hepatocyte growth factor, or both, said method comprising administering to a subject in need of such treatment an effective amount of a modulator molecule of the invention, thereby effectively treating or preventing said cell proliferative disorder. In one embodiment, said proliferative disorder is cancer.

In one aspect, the invention provides a method for inhibiting the growth of a cell, wherein growth of said cell is at least in part dependent upon a growth potentiating effect of HGFA, said method comprising contacting said cell with an effective amount of a modulator molecule of the invention, thereby inhibiting the growth of said cell. In one embodiment, the cell is contacted by HGF expressed by a different cell (e.g., through a paracrine effect).

In one aspect, the invention provides a method for inhibiting the growth of a cell, wherein growth of said cell is at least in part dependent upon a growth potentiating effect of c-met or hepatocyte growth factor, or both, said method comprising contacting said cell with an effective amount of a modulator molecule of the invention, thereby inhibiting the growth of said cell. In one embodiment, the cell is contacted by HGF expressed by a different cell (e.g., through a paracrine effect).

In one aspect, the invention provides a method of therapeutically treating a tumor in a mammal, wherein the growth of said tumor is at least in part dependent upon a growth potentiating effect of HGFA, said method comprising contacting said cell with an effective amount of a modulator molecule of the invention, thereby effectively treating said tumor. In one embodiment, the cell is contacted by HGF expressed by a different cell (e.g., through a paracrine effect).

In one aspect, the invention provides a method of therapeutically treating a tumor in a mammal, wherein the growth of said tumor is at least in part dependent upon a growth potentiating effect of c-met or hepatocyte growth factor, or both, said method comprising contacting said cell with an effective amount of a modulator molecule of the invention, thereby effectively treating said tumor. In one embodiment, the cell is contacted by HGF expressed by a different cell (e.g., through a paracrine effect).

Methods of the invention can be used to affect any suitable pathological state, for example, cells and/or tissues associated with dysregulation of the HGF/c-met signaling pathway, e.g. through increased HGF activity associated with HGFA activation of HGF. In one embodiment, a cell that is targeted in a method of the invention is a cancer cell. For example, a cancer cell can be one selected from the group consisting of a breast cancer cell, a colorectal cancer cell, a lung cancer cell, a papillary carcinoma cell (e.g., of the thyroid gland), a colon cancer cell, a pancreatic cancer cell, an ovarian cancer cell, a cervical cancer cell, a central nervous system cancer cell, an osteogenic sarcoma cell, a renal carcinoma cell, a hepatocellular carcinoma cell, a bladder cancer cell, a prostate cancer cell, a gastric carcinoma cell, a head and neck squamous carcinoma cell, a melanoma cell and a leukemia cell. In one embodiment, a cell that is targeted in a method of the invention is a hyperproliferative and/or hyperplastic cell. In one embodiment, a cell that is targeted in a method of the invention is a dysplastic cell. In yet another embodiment, a cell that is targeted in a method of the invention is a metastatic cell.

Methods of the invention can, further comprise additional treatment steps. For example, in one embodiment, a method further comprises a step wherein a targeted cell and/or tissue (e.g., a cancer cell) is exposed to radiation treatment or a chemotherapeutic agent.

As described herein, HGF/c-met activation is an important biological process the dysregulation of which leads to numerous pathological conditions. Accordingly, in one embodiment of methods of the invention, a cell that is targeted (e.g., a cancer cell) is one in which activation of HGF/c-met is enhanced as compared to a normal cell of the same tissue origin. In one embodiment, a method of the invention causes the death of a targeted cell. For example, contact with a modulator molecule of the invention may result in a cell's inability to signal through the c-met pathway, which results in cell death.

Dysregulation of c-met activation (and thus signaling) can result from a number of cellular changes, including, for example, overexpression of HGF (c-met's cognate ligand) and/or HGFA, and/or increased activation of HGF by HGFA. Accordingly, in some embodiments, a method of the invention comprises targeting a tissue wherein one or more of HGFA, c-met and hepatocyte growth factor, is more abundantly expressed and/or present (e.g., a cancer) as compared to a normal tissue of the same origin. An HGF or c-met-expressing cell can be regulated by HGFA from a variety of sources, i.e. in an autocrine or paracrine manner. For example, in one embodiment of methods of the invention, a targeted cell is contacted/bound by hepatocyte growth factor activated by HGFA expressed in a different cell (e.g., via a paracrine effect). Said different cell can be of the same or of a different tissue origin. In one embodiment, a targeted cell is contacted/bound by HGF activated by HGFA expressed by the targeted cell itself (e.g., via an autocrine effect/loop).

In one aspect, the invention provides compositions comprising one or more modulator molecules of the invention and a carrier. In one embodiment, the carrier is pharmaceutically acceptable.

In one aspect, the invention provides nucleic acids encoding a modulator molecule of the invention. In one embodiment, a nucleic acid of the invention encodes a modulator molecule which is or comprises an antibody or fragment thereof.

In one aspect, the invention provides vectors comprising a nucleic acid of the invention.

In one aspect, the invention provides host cells comprising a nucleic acid or a vector of the invention. A vector can be of any type, for example a recombinant vector such as an expression vector. Any of a variety of host cells can be used. In one embodiment, a host cell is a prokaryotic cell, for example, E. coli. In one embodiment, a host cell is a eukaryotic cell, for example a mammalian cell such as Chinese Hamster Ovary (CHO) cell.

In one aspect, the invention provides methods for making a modulator molecule of the invention. For example, the invention provides a method of making a modulator molecule which is or comprises an antibody (or fragment thereof), said method comprising expressing in a suitable host cell a recombinant vector of the invention encoding said antibody (or fragment thereof), and recovering said antibody.

In one aspect, the invention provides an article of manufacture comprising a container; and a composition contained within the container, wherein the composition comprises one or more modulator molecules of the invention. In one embodiment, the composition comprises a nucleic acid of the invention. In one embodiment, a composition comprising a modulator molecule further comprises a carrier, which in some embodiments is pharmaceutically acceptable. In one embodiment, an article of manufacture of the invention further comprises instructions for administering the composition (for e.g., the modulator molecule) to a subject.

In one aspect, the invention provides a kit comprising a first container comprising a composition comprising one or more modulator molecules of the invention; and a second container comprising a buffer. In one embodiment, the buffer is pharmaceutically acceptable. In one embodiment, a composition comprising a modulator molecule further comprises a carrier, which in some embodiments is pharmaceutically acceptable. In one embodiment, a kit further comprises instructions for administering the composition (for e.g., the modulator molecule) to a subject.

In one aspect, the present disclosure provides a crystal of HGFA complexed with an anti-HGFA antibody that is an allosteric inhibitor of HGFA, and the structural coordinates of the crystal. Coordinates of the crystal structure solved by molecular replacement are listed in Table 7.

In one aspect, the amino acid residues that form a binding site for an allosteric inhibitor of HGFA are identified and are useful, for example, in methods to model the structure of an HGFA allosteric binding site and to identify agents that can bind or fit into the binding site. This use includes the rational design of modulators of HGFA activity. For example, these modulators include ligands that interact with HGFA and modulate met/HGF axis activities, such as binding to HGFA, HGFA activity, cell migration, and Met phosphorylation and signaling.

In one aspect, the present disclosure provides a crystal of HGFA complexed with a Fab40. ΔTrp anti-HGFA antibody, and the structural coordinates of the crystal. Coordinates of the crystal structure solved by molecular replacement are listed in Table 8. In some embodiments, the disclosure provides a crystal structure of HGFA complexed with a Fab40. ΔTrp anti-HGFA antibody, as well as uses of the crystal structure.

In one aspect, the present disclosure provides a crystal of HGFA complexed with KQLR (SEQ ID NO: 21) and a Fab40. ΔTrp anti-HGFA antibody, and the structural coordinates of the crystal. Coordinates of the crystal structure solved by molecular replacement are listed in Table 9. In some embodiments, the disclosure provides a crystal structure of HGFA complexed with KQLR (SEQ ID NO: 21) and a Fab40. ΔTrp anti-HGFA antibody, as well as uses of the crystal structure.

In one aspect, the invention provides HGFA inhibitor RQLR (Arg-Gln-Leu-Arg) peptide (“RQLR” disclosed as SEQ ID NO: 22). In some embodiments, the peptide is an Ac-RQLR-cmk peptidic inhibitor (“RQLR” disclosed as SEQ ID NO: 22).

### BRIEF DESCRIPTION OF THE DRAWINGS

FIG. 1: CDR sequences of anti-HGFA antibodies. The residues are numbered according to the Kabat numbering system (Kabat et al., 1991). The sequence variations between Ab39 and Ab40 are shaded. A single residue deletion (Trp96H) of Fab40 is highlighted in bold. FIG. 1 discloses “CDR-L1” as SEQ ID NOS 1, 1, and 1, “CDR-L2” as SEQ ID NOS 2, 2, and 2, “CDR-L3” as SEQ ID NOS 23, 3, and 3, “CDR-H1” as SEQ ID NOS 24, 24, and 24, “CDR-H2” as SEQ ID NOS 25, 25, and 25, and “CDR-H3” as SEQ ID NOS 26, 26, and 27, all respectively, in order of appearance.

FIG. 2: Inhibition of HGFA enzymatic activity by Ab40. (a) Cleavage of ¹²⁵I-pro-HGF by HGFA in presence of 3-fold serial dilutions of Ab40. The cleavage products HGF α- and β-chain were analyzed by SDS-PAGE (reducing conditions) and subsequently to X-ray film exposure. (b) Partial inhibition of chromogenic substrate, S-2266 hydrolysis (expressed as HGFA fractional activity vᵢ/vₒ) by Ab40 and lack of inhibition by Ab40.ΔTrp. (c) Eadie-Hofstee plot of HGFA inhibition by Ab40 (1 μM-0.004 μM in 3-fold dilution steps; filled diamonds=“no antibody” control) shows competitive inhibition (Vₘₐₓ=0.99 μM pNA/min and Kₘ=0.23 mM for control; Vₘₐₓ^{app}=0.99 μM pNA/min and Kₘ^{app}=0.82 mM for 1 μM Ab40).

FIG. 3: Effects of active site inhibitors on antibody binding to HGFA. (a-b, e-f) Surface plasmon resonance (BIAcore) measurements of binding to immobilized antibodies, Ab40 (a-b) or Ab40.ΔTrp (e-f), after co-injection of HGFA (a,e) or HGFA-KQLR (b, f) complex (“KQLR” disclosed as SEQ ID NO: 21). (c) Competition binding (BIAcore) of HGFA to immobilized Ab40 in presence of different concentrations of KD1. (d) Competition binding ELISA measuring binding of HGFA to biotinylated KD1 in the presence of increasing antibody concentrations.

FIG. 4: Structure of HGFA/Fab40 complex. (a) Surface representation with secondary structure highlighted for the complex between HGFA (ribbon) and Fab40 (light chain: light grey and heavy chain: dark grey). The catalytic triad (His57-Asp 102-Ser195) residues are shown and the 99-loop is highlighted by the arrow. (b) Superposition of HGFA/Fab40 (light grey) structure with HGFA/Fab75 (Wu et al, 2007) (dark grey) showing no significant changes in the conformation of HGFA except for changes in the 99-loop (black). (c) A close view of interactions of CDR-loops (L1-3, H1-3) of Fab40 with HGFA (surface representation). Critical residues involved in the interface interactions are highlighted and the 99-loop is indicated in red. Apart from several hydrogen bonds (dark grey dotted line), a single salt bridge between Asp241 of HGFA and Lys64H of Fab40 is observed.

FIG. 5: HGFA/Fab40 epitope and paratope, with HGFA (light grey) and Fab40 (light chain in light grey, heavy chain in dark grey) (a) Epitope of the Fab40 contact region (dark grey, 4 Å cutoff) on HGFA (light grey). The catalytic triad and the substrate binding subsites S1-S4 are indicated. (b) A different view of the Fab40 contact region on HGFA, which has a partial overlap with a region corresponding to exosite-II in thrombin (green) (c) The HGFA contact region on Fab40 (dotted lines, 4 Å cutoff). The heavy chain is involved in intimate contacts with HGFA and contributes to two-thirds of the total accessible surface area buried on HGFA upon Fab40 binding.

FIG. 6: The three structural snapshots of the 99-loop of HGFA. (a) The ‘allosteric switch’ in the conformation of the 99-loop leads to the formation of a deep hydrophobic pocket (colored in dark grey, residues Ala56, Pro90, Tyr88, Val96, Val105 and Ile107 of HGFA) allowing the binding of Trp96H of Fab40. (b) Size of the hydrophobic pocket (colored in dark grey) in HGFA/Fab40.ΔTrp is severely restricted due to movement of Val96 and other residues lining this pocket. (c) The ‘relaxed’ state conformation of the 99-loop of HGFA as found in other known structures (Shia et al., 2005; Wu et al., 2007). (d) Superposition of the 99-loop of HGFA (light grey and HGFA/Fab40 (dark grey) complex. Conformation transition of the 99-loop upon Fab40 binding, main chain of the 99-loop residues are shifted by >1 Å, while the side chain conformations are dislodged by >2.0. The CDR-H3 loop of Fab40 is highlighted in stick representation (upper). (e) Superposition of the 99-loop of HGFA/Fab40.ΔTrp with the 99-loop of HGFA and with the 99-loop of HGFA/Fab40. The conformation of the 99-loop (light grey) reverts almost back to ‘relaxed’ state in the Fab40.ΔTrp/HGFA complex structure. The CDR-H3 loop of Fab40. ΔTrp is highlighted in stick representation (dark grey). (f) Superposition of the 99-loop of HGFA/Fab40.ΔTrp with the 99-loop of HGFA/Fab40, indicating minor changes in CDR-H3 loop upon deletion of Trp96H of Fab40 (dotted circle).

FIG. 7: The allosteric mechanism. (a) Stereo view of the peptidic inhibitor Ac-KQLR-cmk (“KQLR” disclosed as SEQ ID NO: 21) (sticks embedded in CPK sphere representation in dark grey, lower) is covalently linked to active Ser195 and His57 of HGFA in the HGFA-KQLR/Fab40.ΔTrp complex (“KQLR” disclosed as SEQ ID NO: 21). The P2-Leu packs tightly against Pro99a of the 99-loop (sticks embedded in dots representation in light grey, upper) and a hydrogen bond with Ser99 stabilize the P4-Lys. (b) Stereo view of a model of HGFA-KQLR/Fab40 (“KQLR” disclosed as SEQ ID NO: 21) obtained by from the superposition of HGFA/Fab40 structure with HGFA-KQLR/Fab40.ΔTrp (“KQLR” disclosed as SEQ ID NO: 21) shows the allosteric inhibition is due to the steric clash between Pro99a and Ser99 with P2-Leu of the 99-loop (sticks embedded in dots representation in white). (c) Stereo view of the superposition of HGFA-KQLR/Fab40.ΔTrp (“KQLR” disclosed as SEQ ID NO: 21) with the model of HGFA-KQLR/Fab40 (“KQLR” disclosed as SEQ ID NO: 21) highlighting the critical conformational changes and disruption of hydrogen bond between Ser99 of HGFA and P4-Lys of the inhibitor.

FIG. 8: A cartoon model illustrating the allosteric mechanism of inhibition. In the functionally active state, binding subsites are accessible to substrates and the ‘allosteric switch’ is in the “OFF” state. Fab40 preferentially samples one of the transiently formed conformations and shift the equilibrium away from the functionally active state thus driving the major population of enzyme molecules from the ‘allosteric switch’ “OFF” state to the “ON” state. In contrast, Fab40.ΔTrp which does inhibit enzyme activity might merely bind to the enzyme, without driving a change in state.

FIG. 9: (a) Antibody specificity. In a direct binding ELISA, 96-well plates were coated with 2 mg/ml of HGFA, matriptase (Kirchhofer et al. (2003) J Biol Chem 278:36341-36349), urokinase (American Diagnostica), or factor XIIa (American Diagnostica) and incubated with 10 mg/ml of anti-HGFA antibodies in PBS, 0.05% (v/v) Tween-20 (PBT) buffer. After washing, bound antibodies were detected by addition of anti-human antibody HRP conjugate (diluted 1:2,500 in PBT buffer) and TMB substrate. Absorbance at 450 nm was measured on a microplate reader. (b) Eadie-Hofstee plot of HGFA inhibition by Ab39 (1 μM-0.004 μM in 3-fold dilution steps; filled diamonds=“no antibody” control) shows competitive inhibition (Vₘₐₓ=0.99 μM pNA/min and Kₘ=0.25 mM for control; Vₘₐₓ^{app}=0.97 μM pNA/min and Kₘ^{app}=0.91 mM for 1 μM Ab39).

FIG. 10: Stereo images illustrating the quality of the electron density map (2fofc contoured at 1s). (a) The 99-loop adopts a ‘non-competent’ conformation in HGFA/Fab40 structure. (b) The 99-loop reverts to ‘competent’ conformation in HGFA/Fab40.ΔTrp structure. (c) The covalently bound KQLR (SEQ ID NO: 21) peptide in the HGFA active site does not perturb the binding of Fab40ΔTrp as the 99-loop adopts a ‘competent’ conformation in the HGFA-KQLR/Fab40.ΔTrp (“KQLR” disclosed as SEQ ID NO: 21) structure.

FIG. 11: Chemical structures and hydrogen bond networks at the active site in the HGFA-KQLR/Fab40.ΔTrp complex (“KQLR” disclosed as SEQ ID NO: 21) structure. Peptidic inhibitor KQLR (SEQ ID NO: 21) (center) is covalently bound to Ser195 and His57.

FIG. 12: Superposition of HGFA/Fab40.ΔTrp (light grey) and HGFA-KQLR/Fab40. ΔTrp (darker grey, KQLR (SEQ ID NO: 21)-darkest grey) complex (“KQLR” disclosed as SEQ ID NO: 21) structures. The conformation of the 99-loop is in ‘competent’ conformation as found in other known structures of HGFA.

FIG. 13: Superposition of HGFA/Fab40 and HGFA/KD1 (Shia et al (2005) J Mol Biol 346(5): 1335-49) complex structures. The binding site of Fab40 (dark grey, ribbon) has no direct overlap with the binding site of KD1 (dark grey) but the movement of the 99-loop might cause some steric clash toward the binding of KD1 to HGFA/Fab40 complex.

FIG. 14: Superposition of HGFA/Fab40 (‘non-competent’ conformation of 99-loop (interchangeably termed ‘tense’ conformation; light grey) complex and published HGFA (‘competent’ conformation (interchangeably termed ‘relaxed’ conformation) of 99-loop in black) structure. Steric clashes may occur between the 99-loop residues of HGFA in the ‘competent’ conformation with Trp96H, Ala97H and Trp98H in CDR-H3 loop residues of Fab40.

FIG. 15: Trypsin-like protease domains, aligned using structural homology. Native sequential residue numbering for HGFA appears above the sequences, and chymotrypsinogen numbering appears below the sequences. Chymotrypsinogen numbering includes insertions relative to the chymotrypsinogen sequence denoted with lower case letters (e.g. His60a) and deletions (e.g. there is no residue number 218, and so 217 is followed by 219). Residues 60a, 60b, 60c and 60d follow residue 60, and residues 111a, 111b, 111c and 111d follow residue 111, and residues 170a and 170b follow residue 170. But, residue 99a precedes residue 99, residue 184a precedes residue 184, residue 188a precedes residue 188, and residue 221a precedes residue 221. FIG. 15A discloses SEQ ID NOS 28-36 and FIG. 15B discloses SEQ ID NOS 37-44, all respectively, in order of appearance.

FIG. 16: A. Light chain variable domain sequences of anti-HGFA antibodies. B. Heavy chain variable domain sequences. The residues are numbered according to the Kabat numbering system and Kabat, Chothia and contact CDRs are diagrammatically depicted.

### DETAILED DESCRIPTION OF THE INVENTION

### Definitions

The following definitions are used herein, unless otherwise described:

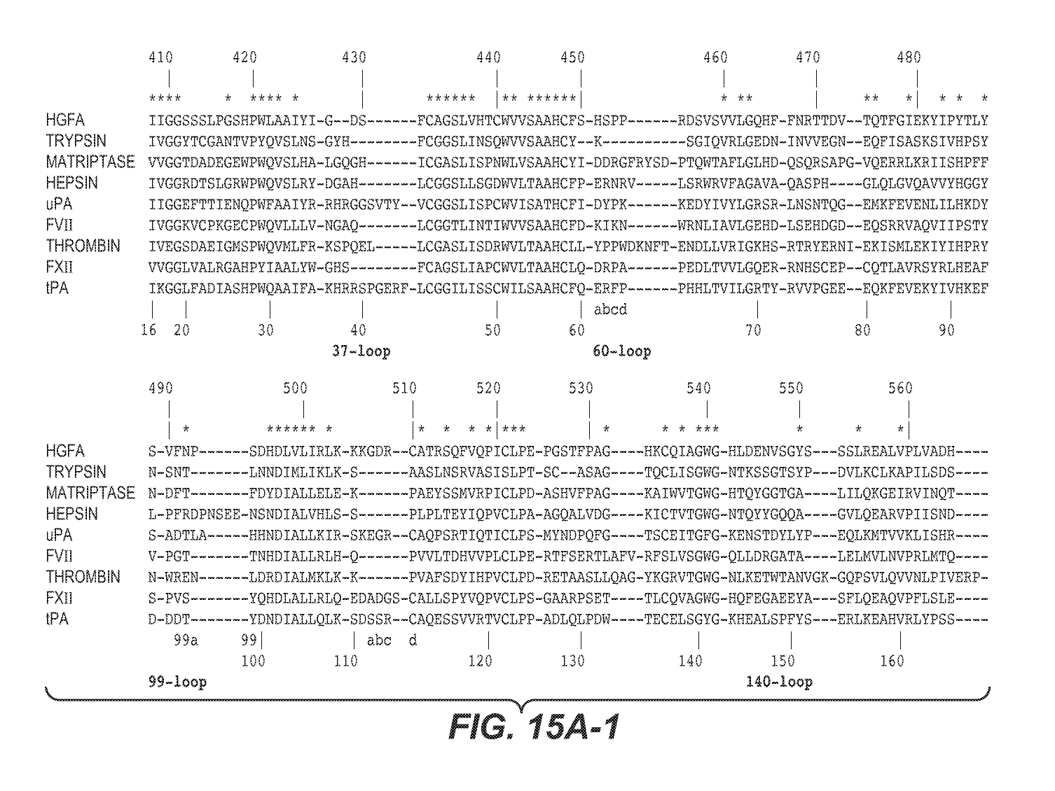

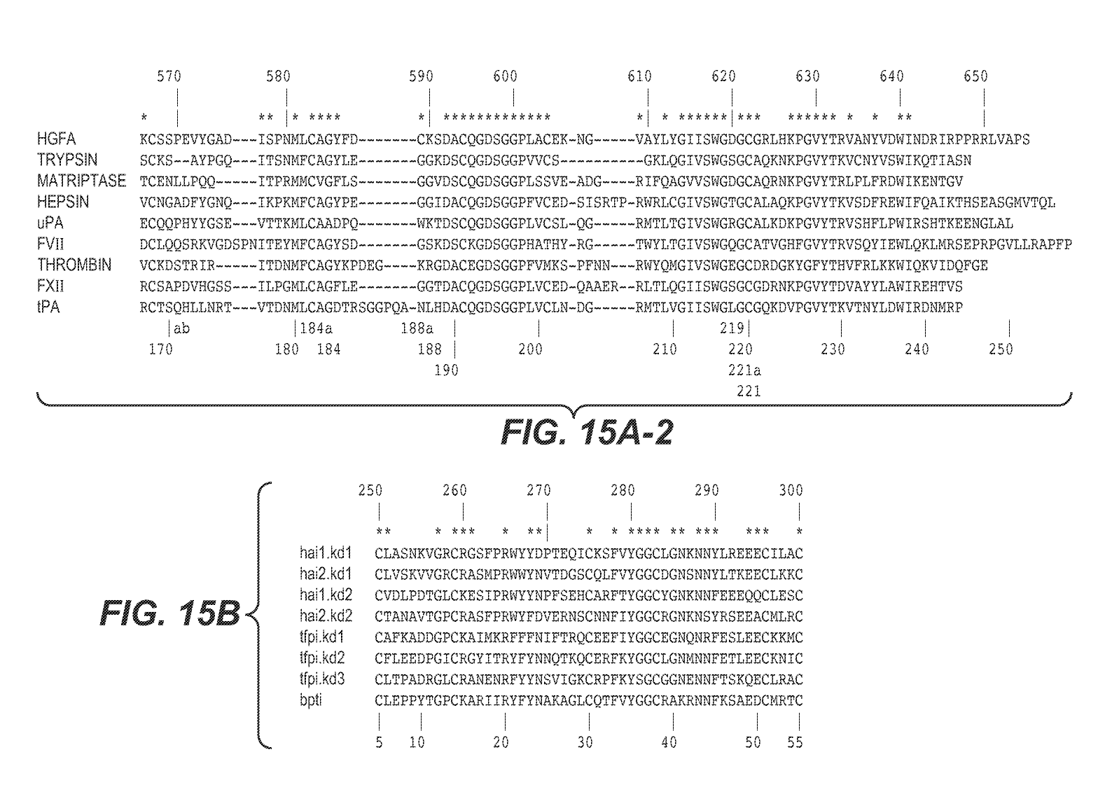

The term “hepatocyte growth factor activator” or “HGFA”, as used herein, refers, unless specifically or contextually indicated otherwise, to any native or variant (whether native or synthetic) HGFA polypeptide that is capable of binding to HGF and/or activating the HGF under conditions that permit such process to occur, for example, conditions that allow for the formation of the two chain form of HGF. The term “wild type HGFA sequence” generally refers to an amino acid sequence found in a naturally occurring HGFA and includes naturally occurring truncated or secreted forms, variant forms (e.g. alternatively spliced forms) and naturally occurring allelic variants. An example of a wild-type HGFA is a polypeptide comprising an amino acid sequence shown in Table 4 (SEQ ID NO:14). The sequence numbering of HGFA is according to SWISS-PROT entry HGFA_HUMAN Accession No. Q04756 and as shown in Table 4. For residues within the protease domain, the alternate numbering scheme derived from chymotrypsinogen is sometimes used. For the interconversion of these two residue numbering schemes, refer to FIG. 15. Generally, throughout this application the numbering system utilized will be identified.

“Activated HGFA” or variations thereof, refers to any HGFA chain having one or more of the conformations that are adopted by wild type HGFA upon conversion of wild type HGFA protein from a single chain form to a 2 chain form. In some embodiments, the conversion results at least in part from cleavage between residue 407 and residue 408 of a HGFA protein. In some embodiments, the conformation refers specifically to the conformation of the protease domain. Activated HGFA may also be generated from fragments of full-length HGFA, such as the 34 kDa form. A 34 kDa form can be generated by cleavage between residues 372 and 373. The HGFA may be isolated from a variety of sources such as human tissue or human plasma or prepared by recombinant or synthetic methods. One embodiment of activated HGFA comprises an amino acid sequence shown in Table 5 (SEQ ID NO:15). (Numbering is that of native HGFA as described herein.)

“HGFA variant” as used herein refers to polypeptide that has a different sequence than a reference polypeptide. In some embodiments, the reference polypeptide is a HGFA polypeptide comprising the sequence shown in Table 4. Variants include “non-naturally” occurring variants. In some embodiments, a variant has at least 80% amino acid sequence identity with the amino acid sequence shown in Table 4 (SEQ ID NO:14). The variants include those polypeptides that have substitutions; additions or deletions. In some embodiments, the variants have the biological activity of binding to the HGF and/or activating it. In other embodiments, the variant can bind to the HGF, but not activate it. Ordinarily, a HGFA variant polypeptide will have at least 80% sequence identity, more preferably will have at least 81% sequence identity, more preferably will have at least 82% sequence identity, more preferably will have at least 83% sequence identity, more preferably will have at least 84% sequence identity; more preferably will have at least 85% sequence identity, more preferably will have at least 86% sequence identity, more preferably will have at least 87% sequence identity, more preferably will have at least 88% sequence identity, more preferably will have at least 89% sequence identity, more preferably will have at least 90% sequence identity, more preferably will have at least 91% sequence identity, more preferably will have at least 92% sequence identity, more preferably will have at least 93% sequence identity, more preferably will have at least 94% sequence identity, more preferably will have at least 95% sequence identity, more preferably will have at least 96% sequence identity, more preferably will have at least 96% sequence identity, more preferably will have at least 97% sequence identity, more preferably will have at least 98% sequence identity, more preferably will have at least 99% sequence identity with a HGFA polypeptide comprising an amino acid sequence comprising the sequence shown in Table 4 or HGFA polypeptide comprising the sequence shown in Table 5.

The term “binding site,” as used herein, refers to a region of a molecule or molecular complex that, as a result of its shape, distribution of electrostatic charge, presentation of hydrogen-bond acceptors or hydrogen-bond donors, and/or distribution of nonpolar regions, favorably associates with a ligand. Thus, a binding site may include or consist of features such as cavities, surfaces, or interfaces between domains. Ligands that may associate with a binding site include, but are not limited to, cofactors, substrates, receptors, agonists, and antagonists. The term binding site includes a functional binding site and/or a structural binding site. A structural binding site can include “in contact” amino acid residues as determined from examination of a three-dimensional structure. “Contact” can be determined using Van der Waals radii of atoms or by proximity sufficient to exclude solvent, typically water, from the space between the ligand and the molecule or molecular complex. In some embodiments, a HGFA residue in contact with KD1 or other substrate or inhibitor is a residue that has one atom within about 5 Å of a KD1 residue. Alternatively, “in contact” residue may be those that have a loss of solvent accessible surface area of at least about 10 Å and, more preferably at least about 50 Å to about 300 Å. Loss of solvent accessible surface can be determined by the method of Lee & Richards (J Mol. Biol. 1971 Feb. 14; 55(3):379-400) and similar algorithms known to those skilled in the art, for instance as found in the SOLV module from C. Broger of F. Hoffman-La Roche in Basel Switzerland.

Some of the “in contact” amino acid residues, if substituted with another amino acid type, may not cause any change in a biochemical assay, a cell-based assay, or an in vivo assay used to define a functional binding site but may contribute to the formation of a three dimensional structure. A functional binding site includes amino acid residues that are identified as binding site residues based upon loss or gain of function, for example, loss of binding to ligand upon mutation of the residue. In some embodiments, the amino acid residues of a functional binding site are a subset of the amino acid residues of the structural binding site.

The term “HGFA allosteric binding site” includes all or a portion of a molecule or molecular complex whose shape is sufficiently similar to at least a portion of a binding site on HGFA for an allosteric inhibitor as to be expected to other allosteric inhibitors. A structurally equivalent ligand binding site is defined by a root mean square deviation from the structure coordinates of the backbone atoms of the amino acids that make up binding sites in HGFA for anti-HGFA antibody Ab40 of at most about 0.70 Å, preferably about 5 Å. In some embodiments, an allosteric binding site for an allosteric inhibitor of HGFA comprises, consists essentially of, or consists of at least one amino acid residue corresponding to residues 446, 449, 450, 452, 453, 455, 480, 481, 482, 483, 484, 485, 486, 487, 488, 489, 490, 491, 496, 499, 501, 578, 579, 580, 636, 637, 640, 643, 644, or mixtures thereof. In some embodiments, an allosteric binding site for the HGFA for an allosteric inhibitor of HGFA comprises, consists essentially of, or consists of at least one amino acid residue corresponding to residues 449, 450, 452, 482, 484, 485, 486, 487, 488, 489, 490, 491, or mixtures thereof. In some embodiments, an allosteric binding site for the HGFA for an allosteric inhibitor of HGFA comprises, consists essentially of, or consists of at least one amino acid residue corresponding to residues 446, 482, 484, 490, 499, 501, or mixtures thereof. A structurally equivalent binding site is defined by a root mean square deviation from the structure coordinates of the backbone atoms of the amino acids that make up a binding site of HGFA of at most about 0.70 Å, preferably about 0.5 Å.

“Crystal” as used herein, refers to one form of a solid state of matter in which atoms are arranged in a pattern that repeats periodically in three-dimensions, typically forming a lattice.

“Complementary or complement” as used herein, means the fit or relationship between two molecules that permits interaction, including for example, space, charge, three-dimensional configuration, and the like.

The term “corresponding” or “corresponds” refers to an amino acid residue or amino acid sequence that is found at the same position or positions in a sequence when the amino acid position or sequences are aligned with a reference sequence. In some embodiments, the reference sequence is a fragment of the HGFA having a sequence shown in table 4 or 5. It will be appreciated that when the amino acid position or sequence is aligned with the reference sequence the numbering of the amino acids may differ from that of the reference sequence.

“Heavy atom derivative”, as used herein, means a derivative produced by chemically modifying a crystal with a heavy atom such as Hg, Au, or a halogen.

“Structural homolog” of HGFA as used herein refers to a protein that contains one or more amino acid substitutions, deletions, additions, or rearrangements with respect to the amino acid sequence of HGFA, but that, when folded into its native conformation, exhibits or is reasonably expected to exhibit at least a portion of the tertiary (three-dimensional) structure of the HGFA. In some embodiments, a portion of the three dimensional structure refers to structural domains of the HGFA, including the N-terminal fibronectin type II domain, either of two EGF-like domains, fibronectin type I domain, Kringle domain and C terminal trypsin-like serine protease domain, or combinations thereof. Homolog tertiary structure can be probed, measured, or confirmed by known analytic or diagnostic methods, for example, X-ray, NMR, circular dichroism, a panel of monoclonal antibodies that recognize native HGFA, and like techniques. For example, structurally homologous molecules can have substitutions, deletions or additions of one or more contiguous or noncontiguous amino acids, such as a loop or a domain. Structurally homologous molecules also include “modified” HGFA molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and like modifications.

“Ligand”, as used herein, refers to an agent or compound that associates with a binding site on a molecule, for example, HGFA allosteric binding sites, and may be an antagonist or agonist of HGFA activity. Ligands include molecules that mimic Fab40 binding to HGFA.

“Compound” refers to molecule that associates with the HGFA or a pharmaceutically acceptable salt, ester, amide, prodrug, isomer, or metabolite, thereof. “Pharmaceutically acceptable salt” refers to a formulation of a compound that does not compromise the biological activity and properties of the compound. Pharmaceutical salts can be obtained by reacting a binding-active compound of the disclosure with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. “Prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transport across a cell membrane where water solubility is detrimental to mobility but which then is metabolically hydrolyzed to the carboxylic acid, the active entity, once inside the cell where water solubility is beneficial. A further example of a prodrug might be a short peptide (polyaminoacid) bonded to an acid group wherein the peptide is metabolized to yield the active moiety.

“Molecular complex”, as used herein, refers to a combination of bound substrate or ligand with polypeptide, such as an antibody bound to HGFA, or a ligand bound to HGFA.

“Machine-readable data storage medium”, as used herein, means a data storage material encoded with machine-readable data, wherein a machine programmed with instructions for using such data and is capable of displaying data in the desired format, for example, a graphical three-dimensional representation of molecules or molecular complexes.

“Scalable,” as used herein, means the increasing or decreasing of distances between coordinates (configuration of points) by a scalar factor while keeping the angles essentially the same.

“Space group symmetry”, as used herein, means the whole symmetry of the crystal that combines the translational symmetry of a crystalline lattice with the point group symmetry. A space group is designated by a capital letter identifying the lattice type (P, A, F, etc.) followed by the point group symbol in which the rotation and reflection elements are extended to include screw axes and glide planes. Note that the point group symmetry for a given space group can be determined by removing the cell centering symbol of the space group and replacing all screw axes by similar rotation axes and replacing all glide planes with mirror planes. The point group symmetry for a space group describes the true symmetry of its reciprocal lattice.

“Unit cell”, as used herein, means the atoms in a crystal that are arranged in a regular repeating pattern, in which the smallest repeating unit is called the unit cell. The entire structure can be reconstructed from knowledge of the unit cell, which is characterized by three lengths (a, b and c) and three angles (α, β and γ). The quantities a and b are the lengths of the sides of the base of the cell and γ is the angle between these two sides. The quantity c is the height of the unit cell. The angles α and β describe the angles between the base and the vertical sides of the unit cell.

“X-ray diffraction pattern” means the pattern obtained from X-ray scattering of the periodic assembly of molecules or atoms in a crystal. X-ray crystallography is a technique that exploits the fact that X-rays are diffracted by crystals. X-rays have the proper wavelength (in the Ångström (Å) range, approximately 10⁻⁸ cm) to be scattered by the electron cloud of an atom of comparable size. Based on the diffraction pattern obtained from X-ray scattering of the periodic assembly of molecules or atoms in the crystal, the electron density can be reconstructed. Additional phase information can be extracted either from the diffraction data or from supplementing diffraction experiments to complete the reconstruction (the phase problem in crystallography). A model is then progressively built into the experimental electron density, refined against the data to produce an accurate molecular structure.

X-ray structure coordinates define a unique configuration of points in space. Those of skill in the art understand that a set of structure coordinates for a protein or a protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a configuration of points can be defined by increasing or decreasing the distances between coordinates by a scalar factor, while keeping the angles essentially the same.

“Crystal structure” generally refers to the three-dimensional or lattice spacing arrangement of repeating atomic or molecular units in a crystalline material. The crystal structure of a crystalline material can be determined by X-ray crystallographic methods, see for example, “Principles of Protein X-Ray Crystallography,” by Jan Drenth, Springer Advanced Texts in Chemistry, Springer Verlag; 2nd ed., February 1999, ISBN: 0387985875, and “Introduction to Macromolecular Crystallography,” by Alexander McPherson, Wiley-Liss, Oct. 18, 2002, ISBN: 0471251224.

“Allosteric regulation” and like terms refers to regulation of a functional site of HGFA by way of large scale conformational changes in the shape of HGFA which can be caused by, for example, the binding of a regulatory molecule elsewhere (i.e., other than at the functional site) in the HGFA molecule. An “allosteric regulator” or signal molecule is any molecule capable of effecting such allosteric regulation or signaling in the HGFA molecule. An allosteric regulator can be either positive (an activator) or negative (an inhibitor) of HGFA activity. Allosteric regulation of HGFA activity can involve cooperativity, which requires cooperative interaction of its multiple protein subunits, or allosteric regulation of HGFA activity can occur without cooperativity in any of the protein subunits.

The terms “antibody” and “immunoglobulin” are used interchangeably in the broadest sense and include monoclonal antibodies (for e.g., full length or intact monoclonal antibodies), polyclonal antibodies, multivalent antibodies, multispecific antibodies (e.g., bispecific antibodies so long as they exhibit the desired biological activity) and may also include certain antibody fragments (as described in greater detail herein). An antibody can be human, humanized and/or affinity matured.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For e.g., such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “blocking” antibody or an “antagonist” antibody is one which inhibits or reduces biological activity of the antigen it binds. Preferred blocking antibodies or antagonist antibodies substantially or completely inhibit the biological activity of the antigen.

A “disorder” is any condition that would benefit from treatment with a substance/molecule or method of the invention. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic and other angiogenesis-related disorders.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

A “growth inhibitory agent” when used herein refers to a compound or composition which inhibits growth of a cell whose growth is dependent upon HGF/c-met activation either in vitro or in vivo. Thus, the growth inhibitory agent may be one which significantly reduces the percentage of HGF/c-met-dependent cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

### MODES FOR CARRYING OUT THE INVENTION

The invention provides methods, composition, kits and articles of manufacture for modulating hepatocyte growth factor activator (HGFA) function, thereby modulating physiological effects of HGFA activity. Described herein is the identification of a hydrophobic binding pocket, termed an allosteric binding site, of HGFA that favorably associates with an allosteric inhibitor, whereby HGFA enzymatic activity is altered (inhibited) from a location that is remote from the catalytic site. Structural and kinetic studies described herein provide a detailed view of how an allosteric inhibitor inhibits HGFA catalysis. The data described herein provide a basis for design of HGFA antagonists capable of inhibiting wild type HGFA activity. These antagonists can be used as advantageous therapeutic agents for treating pathological conditions wherein reduced HGF/c-met biological activity is desirable. Methods and compositions of the invention are based generally on these findings, which are described in greater detail below.

The present disclosure also includes a crystalline form and a crystal structure of HGFA complexed with an anti-HGFA antibody, Fab40, and methods of using the HGFA:Fab40 crystal structure and structural coordinates to identify homologous proteins and to design or identify agents that can modulate the function of HGFA or the HGFA-Fab40 complex. The present disclosure also includes the three-dimensional configuration of points derived from the structure coordinates of at least a portion of an HGFA molecule or molecular complex, as well as structurally equivalent configurations, as described below. The three-dimensional configuration includes points derived from structure coordinates of, e.g., the HGFA:Fab40 complex, representing the locations of a plurality of the amino acids defining the HGFA allosteric binding site.

In some embodiments, the three-dimensional configuration includes points derived from structure coordinates representing the locations of the backbone atoms of a plurality of amino acids defining the HGFA-Fab40 complex or the HGFA allosteric binding site of, e.g., the HGFA:Fab40 complex. Alternatively, the three-dimensional configuration includes points derived from structure coordinates representing the locations of the side chain and the backbone atoms (other than hydrogens) of a plurality of the amino acids defining the HGFA-Fab40 complex.

The present disclosure also includes a crystalline form and a crystal structure of HGFA complexed with a mutated anti-HGFA antibody, Fab40.ΔTrp, and methods of using the HGFA:Fab40.ΔTrp crystal structure and structural coordinates to identify homologous proteins and to design or identify agents that can modulate the function of HGFA or the HGFA:Fab40.ΔTrp complex. The present disclosure also includes the three-dimensional configuration of points derived from the structure coordinates of at least a portion of an HGFA molecule or molecular complex, as well as structurally equivalent configurations, as described below.

The present disclosure also includes a crystalline form and a crystal structure of HGFA complexed with an mutated anti-HGFA antibody, Fab40.ΔTrp and synthetic HGFA substrate, KQLR (SEQ ID NO: 21), and methods of using the HGFA:KQLR:Fab40. ΔTrp complex (“KQLR” disclosed as SEQ ID NO: 21) crystal structure and structural coordinates to identify homologous proteins and to design or identify agents that can modulate the function of HGFA or the HGFA:KQLR:Fab40. ΔTrp complex (“KQLR” disclosed as SEQ ID NO: 21). The present disclosure also includes the three-dimensional configuration of points derived from the structure coordinates of at least a portion of an HGFA molecule or HGFA:KQLR:Fab40. ΔTrp molecular complex (“KQLR” disclosed as SEQ ID NO: 21), as well as structurally equivalent configurations, as described below.

In some embodiments, the three-dimensional configuration also includes the three-dimensional configuration of points identifying other structural features of an HGFA, such as S1 subsite, S2 subsite, S3/4 subsite, S1′, S2′, S3′, a HGFA binding site for KD1, and/or a HGFA binding site for a substrate or inhibitor. In some embodiments, the S1 subsite comprises at least one amino acid residue in a position of HGFA selected from the group consisting of 592(189), 593(190), 594(191), 595(192), 597(194), 616(213), 618(215), 619(216), 620(217), 621(219), 622(220), 631(228), and mixtures thereof. In some embodiments, the S2 subsite comprises at least one amino acid residue in a position of HGFA selected from the group consisting of 447(57), 493(99a), 494(99), 497(102), and mixtures thereof. In some embodiments, the S3/4 subsite that comprises at least one amino acid residue in a position of HGFA selected from the group consisting of 492(98), 493(99a), 494(99), 576(175), 581(180), 618(215), and mixtures thereof. In some embodiments, the S1′ subsite comprises at least one amino acid residue corresponding to the residue in a position of HGFA selected from the group consisting of 432(42), 447(57), 448(58), 598(195), and mixtures thereof. In some embodiments, the S2′ subsite comprises at least one amino acid residue in a position of HGFA selected from the group consisting of 430(40), 431(41), 542(143), 550(151), 595(192), 596(193), and mixtures thereof. In some embodiments, the S3′ subsite comprises at least one amino acid residue in a position of HGFA selected from the group consisting of 427 (35), 430(40), 432(42), 447(57), 448(58), 449(59), 451(60a), and mixtures thereof. Numbering is native HGFA with chymotrypsinogen numbering in parenthesis. In some embodiments, an HGFA binding site for KD1 comprises, consists essentially or consists of at least one residue is in a position of HGFA selected from the group consisting of 429(39), 430(40), 431(41), 447(58), 448(58), 451(60a), 494(99), 548(149), 550(151), 592(189), 593(190), 595(192), 596(193), 597(194), 598(195), 617(214), 618(215), 619(216), 621(219), and mixtures thereof.

The disclosure also includes the scalable three-dimensional configuration of points derived from structure coordinates of molecules or molecular complexes that are structurally homologous to HGFA, HGFA:Fab40, HGFA:Fab40. ΔTrp, or HGFA:KQLR:Fab40. ΔTrp complexes (“KQLR” disclosed as SEQ ID NO: 21), as well as structurally equivalent configurations. Structurally homologous molecules or molecular complexes are defined below. Advantageously, structurally homologous molecules can be identified using the structure coordinates of the HGFA, HGFA:Fab40, HGFA:Fab40. ΔTrp, or HGFA:KQLR:Fab40. ΔTrp complexes (“KQLR” disclosed as SEQ ID NO: 21) or extracellular fragment of HGFA according to a method of the disclosure.

The configurations of points in space derived from structure coordinates according to the disclosure can be visualized as, for example, a holographic image, a stereodiagram, a model, or a computer-displayed image, and the disclosure thus includes such images, diagrams or models.

The crystal structure and structural coordinates can be used in methods, for example, for obtaining structural information of a related molecule, and for identifying and designing agents that modulate HGFA, or HGFA:Fab40, HGFA:Fab40. ΔTrp, or HGFA:KQLR:Fab40. ΔTrp complex (“KQLR” disclosed as SEQ ID NO.: 21) activity.

### 1. HGFA Polypeptides, Polynucleotides and Variants Thereof

Native or wild-type HGFA are those polypeptides that have a sequence of a polypeptide obtained from nature. Native or wild-type polypeptides include naturally occurring variants, secreted or truncated forms. An embodiment of wild type HGFA comprises a sequence of SEQ ID NO:14 shown in Table 4. In some embodiments, a polypeptide comprising, consisting essentially or consisting of the polypeptide show in Table 5 (SEQ ID NO:15) is provided.

The structure of HGFA and HGFA in complex with substrate KD1 is disclosed in, e.g. WO2006/063300; Wu, Y. et al. PNAS 104, 19784-19789 (2007); Shia, S. et al. J Mol Biol 346, 1335-49 (2005).

Hepatocyte growth factor activator is secreted as a 96 KDa zymogen (proHGFA) with a domain structure comprising a N terminal fibronectin type II domain, an EGF-like domain, a fibronectin type I domain, another EGF-like domain, a kringle domain and a C terminal trypsin homology serine protease domain. Cleavage at a kallikrein sensitive site between Arg372 and Val373 produces a short 34 kDa form that lacks the first 5 domains. Both 96 kDa and 34 kDa forms can be cleaved between residues Arg407 and Ile408 to produce activated HGFA. Activated wild type HGFA binds to and cleaves proHGF into activated HGF. (Numbering is that of native HGFA).

The present disclosure also includes a polypeptide comprising, consisting essentially of, or consisting of a portion or fragment of the HGFA. The numbering of HGFA will be according to either of two systems. The numbering according to SWISS-PROT entry HGFA_HUMAN starts at 1 and ends at 655 (as shown in Table 4 and SWISS-PROT Accession No. Q04756). Reference herein to HGFA residue numbers between 16 and 243 are according to the numbering system by analogy to chymotrypsinogen. Nowhere is reference made to a residue numbered from 16 to 243 according to the HGFA_HUMAN scheme. Therefore, reference to HGFA residues numbered from 16 to 243 employ the chymotrypsinogen scheme, and reference to HGFA residues numbered less than 16 or greater than 243 employ the numbering in HGFA_HUMAN. Conversion between the two schemes can be made by reference to FIG. 15. The fragments preferably lack one or more of the first 5 domains and retain the trypsin-like protease domain. An embodiment of a polypeptide fragment comprises, consists essentially of, or consists of any of amino acid residue starting from amino acid 373 residue to amino acid position 408 and ending at amino acid residue 655 or residues corresponding to those positions, e.g., as shown in Table 5. In some embodiments, the polypeptide portion has the ability to bind to HGF and activate it.

The present disclosure also includes variants of HGFA. Variants include those polypeptides that have amino acid substitutions, deletions, and additions. Amino acid substitutions can be made for example to replace cysteines and eliminate formation of disulfide bonds. Amino acid substitutions can also be made to change proteolytic cleavage sites. Other variants can be made at the HGFA allosteric binding site or the HGFA binding site for HGF or KD1 (See WO2006/063300). In other embodiments, the variants of the HGFA bind HGF or KD1 with the same or higher affinity than the wild type HGFA. In some embodiments, the variants of the HGFA bind an allosteric HGFA inhibitor (eg Fab40) with the same or higher affinity that the wild type HGFA.

### Fusion Proteins

HGFA polypeptides, variants, or structural homolog or portions thereof, may be fused to a heterologous polypeptide or compound. The heterologous polypeptide is a polypeptide that has a different function than that of the HGFA. Examples of heterologous polypeptide include polypeptides that may act as carriers, may extend half life, may act as epitope tags, may provide ways to detect or purify the fusion protein. Heterologous polypeptides include KLH, albumin, salvage receptor binding epitopes, immunoglobulin constant regions, and peptide tags. Peptide tags useful for detection or purification include FLAG, gD protein, polyhistidine tags, hemagluthinin from influenza virus, T7 tag, S tag, Strep tag, chloramiphenicol acetyl transferase, biotin, glutathione-S transferase, green fluorescent protein and maltose binding protein. Compounds that can be combined with the HGFA, variants or structural homolog or portions thereof, include radioactive labels, protecting groups, and carbohydrate or lipid moieties.

### Polynucleotides, Vectors and Host Cells

HGFA, variants or fragments thereof can be prepared by introducing appropriate nucleotide changes into DNA encoding HGFA, or by synthesis of the desired polypeptide variants.

Polynucleotide sequences encoding the polypeptides described herein can be obtained using standard recombinant techniques. Desired polynucleotide sequences may be isolated and sequenced from appropriate source cells. Alternatively, polynucleotides can be synthesized using nucleotide synthesizer or PCR techniques. Once obtained, sequences encoding the polypeptides or variant polypeptides are inserted into a recombinant vector capable of replicating and expressing heterologous polynucleotides in a host cell. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication (in particular when the vector is inserted into a prokaryotic cell), a selection marker gene, a promoter, a ribosome binding site (RBS), a signal sequence, the heterologous nucleic acid insert and a transcription termination sequence.

In general, plasmid vectors containing replicon and control sequences, which are derived from a species compatible with the host cell are used in connection with these hosts. The vector ordinarily carries a replication site, as well as marking sequences, which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

Either constitutive or inducible promoters can be used in the present invention, in accordance with the needs of a particular situation, which can be ascertained by one skilled in the art. A large number of promoters recognized by a variety of potential host cells are well known. The selected promoter can be operably linked to cistron DNA encoding a polypeptide described herein by removing the promoter from the source DNA via restriction enzyme digestion and inserting the isolated promoter sequence into the vector of choice. Both the native promoter sequence and many heterologous promoters may be used to direct amplification and/or expression of the target genes. However, heterologous promoters are preferred, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the polypeptides or variant polypeptides (Siebenlist et al. (1980) Cell 20: 269) using linkers or adaptors to supply any required restriction sites.

In embodiments, each cistron within a recombinant vector comprises a secretion signal sequence component that directs translocation of the expressed polypeptides across a membrane. In general, the signal sequence may be a component of the vector, or it may be a part of the polypeptide encoding DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

Prokaryotic host cells suitable for expressing polypeptides include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. Preferably, gram-negative cells are used. Preferably the host cell should secrete minimal amounts of proteolytic enzymes, and additional protease inhibitors may desirably be incorporated in the cell culture.

Besides prokaryotic host cells, eukaryotic host cell systems are also well established in the art. Examples of invertebrate cells include insect cells such as Drosophila S2 and Spodoptera Sf9, as well as plants and plant cells. Examples of useful mammalian host cell lines include Chinese hamster ovary (CHO) and COS cells. More specific examples include monkey kidney CV1 line transformed by SV40 (COS-7, ATCC CRL 1651); Chinese hamster ovary cells/−DHFR(CHO, Urlaub and Chasin, Proc. Natl. Acad. Sci. USA, 77:4216 (1980)); mouse sertoli cells (TM4, Mather, Biol. Reprod., 23:243-251 (1980)); and mouse mammary tumor (MMT 060562, ATCC CCL51).

### Polypeptide Production

Host cells are transformed or transfected with the above-described expression vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In preferred embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. For example, if a PhoA promoter is used for controlling transcription, the transformed host cells may be cultured in a phosphate-limiting medium for induction. A variety of other inducers may be used, according to the vector construct employed, as is known in the art.

Eukaryotic host cells are cultured under conditions suitable for expression of the HGFA and/or KD polypeptides. The host cells used to produce the polypeptides may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in one or more of Ham et al., 1979, Meth. Enz. 58:44, Barnes et al., 1980, Anal. Biochem. 102: 255, U.S. Pat. No. 4,767,704, U.S. Pat. No. 4,657,866, U.S. Pat. No. 4,927,762, U.S. Pat. No. 4,560,655, or U.S. Pat. No. 5,122,469, WO 90/103430, WO 87/00195, and U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES™), nucleotides (such as adenosine and thymidine), antibiotics (such as GENTAMYCIN™), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

Polypeptides described herein expressed in a host cell may be secreted into and/or recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated there from. Cells may be removed from the culture and the culture supernatant being filtered and concentrated for further purification of the proteins produced. The expressed polypeptides can be further isolated and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable antigen immobilized on a matrix and Western blot assay.

Polypeptides that are produced may be purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

Antibody Production

For recombinant production of an antibody, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

### Generating Antibodies Using Prokaryotic Host Cells:

### Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB⁻ strains) provide cytoplasm conditions that are favorable for disulfide bond formation, thereby permitting proper folding and assembly of expressed protein subunits. Proba and Pluckthun Gene, 159:203 (1995).

The present invention provides an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the invention.

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 ΔfhuA (ΔtonA) ptr3 lac Iq lacL8 ΔompTΔ(nmpc-fepE) degP41 kan^{R} (U.S. Pat. No. 5,639,635). Other strains and derivatives thereof, such as E. coli 294 (ATCC 31,446), E. coli B, E. coli_{λ} 1776 (ATCC 31,537) and E. coli RV308(ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

### Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. Preferably, the phosphate-limiting medium is the C.R.A.P medium (see, for e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

### Antibody Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the full length antibody products of the invention. Protein A is a 41 kD cell wall protein from Staphylococcus aureas which binds with a high affinity to the Fc region of antibodies. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

### Generating Antibodies Using Eukaryotic Host Cells:

The vector components generally include, but are not limited to, one or more of the following: a signal sequence, an origin of replication, one or more marker genes, an enhancer element, a promoter, and a transcription termination sequence.

(i) Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

(ii) Origin of Replication

Generally, an origin of replication component is not needed for mammalian expression vectors. For example, the SV40 origin may typically be used only because it contains the early promoter.

(iii) Selection Gene Component Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

(iv) Promoter Component

Expression and cloning vectors usually contain a promoter that is recognized by the host organism and is operably linked to the antibody polypeptide nucleic acid. Promoter sequences are known for eukaryotes. Virtually alleukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of DNA encoding the antibody polypeptide of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody polypeptide-encoding sequence, but is preferably located at a site 5′ from the promoter.

(vi) Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Activity Assays

The antibodies of the present invention can be characterized for their physical/chemical properties and biological functions by various assays known in the art.

The purified immunoglobulins can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In certain embodiments of the invention, the immunoglobulins produced herein are analyzed for their biological activity. In some embodiments, the immunoglobulins of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays.

### Humanized Antibodies

The present invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

### 2. Crystals and Crystal Structures

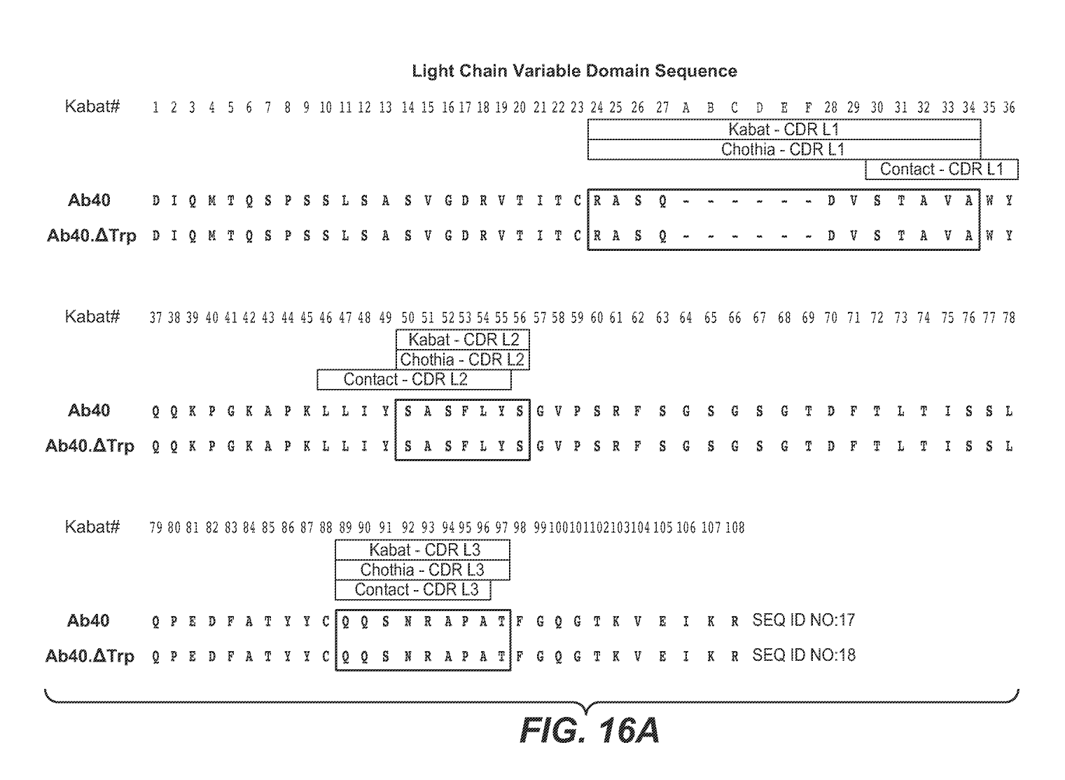

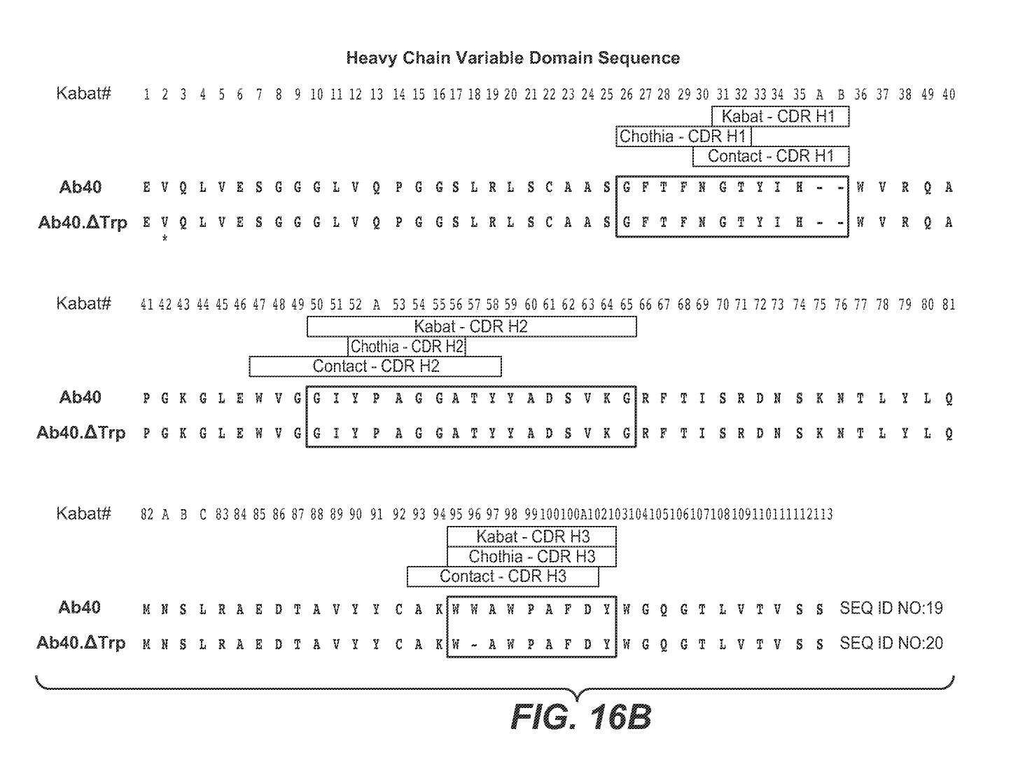

The present disclosure provides crystals of an HGFA:anti-HGFA antibody (Fab40) complex as well as the crystal structure of HGFA:Fab40 as determined therefrom. In some embodiments, the crystals are formed from an HGFA sequence comprising sequence shown in Table 5 and a Fab40 antibody comprising light chain and heavy chain variable region amino acid sequences shown in FIGS. 16A and 16B, respectively. In some embodiments, the Fab40 antibody comprises a light chain comprising sequence DIQMTQSPSSLSASVGDRVTITCRASQDVSTAVAWYQQKPGKAPKLLIYSASFLY SGVPSRFSGSGSGTDFTLTISSLQPEDFATYYCQQSNRAPATFGQGTKVEIKRTVA APSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGEC (SEQ ID NO:9) and a heavy chain comprising sequence EVQLVESGGGLVQPGGSLRLSCAASGFTINGTYIHWVRQAPGKGLEWVGGIYPA GGATYYADSVKGRFTISADTSKNTAYLQMNSLRAEDTAVYYCAKWWAWPAFD YWGQGTLVTVSSASTKGPSVFPLAPSSKSTSGGTAALGCLVKDYFPEPVTVSWN SGALTSGVHTFPAVLQSSGLYSLSSVVTVPSSSLGTQTYICNVNHKPSNTKVDKK VEPKSCDKTH (SEQ ID NO10).

The resulting crystals diffracted to 2.35 Å resolution (Table 2). The structure was refined to an R-value of 0.198 (Rfree0.261) with good geometry.

In a specific embodiment, the structure of HGFA complexed with a Fab40 antibody was solved by molecular replacement with the program PHASER. The crystals belonged to space group P₁ with cell parameters of a=38.94 Å, b=48.93 Å and c=96.03 Å and α=98.10 Å β=95.01 Å and γ=103.89 Å.

The present disclosure also provides crystals of an HGFA:Fab40. ΔTrp complex as well as the crystal structure of HGFA:Fab40. ΔTrp as determined therefrom. In some embodiments, the crystals are formed from an HGFA sequence comprising sequence shown in Table 5 and a Fab40. ΔTrp sequence comprising light chain and heavy chain variable region amino acid sequences shown in FIGS. 16A and 16B, respectively. In some embodiments, the Fab40. ΔTrp antibody comprises a light chain comprising sequence

DIQMTQSPSSLSASVGDRVTITCRASQDVSTAVAWYQQKPGKAPKLLIYS ASFLYSGVPSRFSGSGSGTDFTLTISSLQPEDFATYYCQQSNRAPATFGQGTKVEI KRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC (SEQ ID NO:11), and a heavy chain comprising sequence

EVQLVESGGGLVQPGGSLRLSCAASGFTINGTYIHWVRQAPGKGLEWVG GIYPAGGATYYADSVKGRFTISADTSKNTAYLQMNSLRAEDTAVYYCAKWAWP AFDYWGQGTLVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKVD KKVEPKSCDKTH (SEQ ID NO:12).

The resulting crystals diffracted to 2.90 Å resolution (Table 2). The structure was refined to an R-value of 0.209 (Rfree 0.297) with good geometry.

In a specific embodiment, the structure of HGFA complexed with a Fab40. ΔTrp antibody was solved by molecular replacement with the program PHASER. The crystals belonged to space group P2₁ with cell parameters of a=72.36 Å, b=89.53 Å and c=118.47 Å and β=95.01 Å.

The present disclosure also provides crystals of an HGFA:KQLR:Fab40. ΔTrp complex (“KQLR” disclosed as SEQ ID NO: 21) as well as the crystal structure of HGFA:KQLR:Fab40. ΔTrp (“KQLR” disclosed as SEQ ID NO: 21) determined therefrom. The present disclosure also provides crystals of an HGFA:KQLR:Fab40. ΔTrp complex (“KQLR” disclosed as SEQ ID NO: 21) as well as the crystal structure of HGFA:KQLR:Fab40. ΔTrp (“KQLR” disclosed as SEQ ID NO: 21) as determined therefrom. In some embodiments, the crystals are formed from an HGFA sequence comprising sequence shown in Table 5 and a Fab40. ΔTrp sequence comprising light chain and heavy chain variable region amino acid sequences shown in FIGS. 16A and 16B, respectively, and a KQLR peptide (“KQLR” disclosed as SEQ ID NO: 21) (Lys-Gln-Leu-Arg (SEQ ID NO: 21)). In some embodiments, the Fab40. ΔTrp antibody comprises a light chain comprising sequence

DIQMTQSPSSLSASVGDRVTITCRASQDVSTAVAWYQQKPGKAPKLLIYS ASFLYSGVPSRFSGSGSGTDFTLTISSLQPEDFATYYCQQSNRAPATFGQGTKVEI KRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC (SEQ ID NO:11), and a heavy chain comprising sequence

EVQLVESGGGLVQPGGSLRLSCAASGFTINGTYIHWVRQAPGKGLEWVG GIYPAGGATYYADSVKGRFTISADTSKNTAYLQMNSLRAEDTAVYYCAKWAWP AFDYWGQGTLVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKVD KKVEPKSCDKTH (SEQ ID NO:12).

The resulting crystals diffracted to 2.8 Å resolution (Table 2). The structure was refined to an R-value of 0.198 (Rfree 0.277) with good geometry.

In a specific embodiment, the structure of HGFA:KQLR:Fab40. ΔTrp (“KQLR” disclosed as SEQ ID NO: 21) antibody was solved by molecular replacement with the program PHASER. The crystals belonged to space group C222₁ with cell parameters of a=80.36 Å, b=147.89 Å and c=146.37 Å.

The crystals are useful to provide the crystal structure and to provide a stable form of the molecule for storage.

Each of the constituent amino acids of HGFA:Fab40, HGFA:Fab40. ΔTrp, or HGFA:KQLR:Fab40. ΔTrp (“KQLR” disclosed as SEQ ID NO: 21) is defined by a set of structure coordinates as set forth in Tables 7, 8 and 9, respectively. The term “structure coordinates” refers to Cartesian coordinates derived from mathematical equations related to the patterns obtained on diffraction of a monochromatic beam of X-rays by the atoms (scattering centers) of a HGFA, HGFA:Fab40, HGFA:Fab40. ΔTrp, or HGFA:KQLR:Fab40. ΔTrp (“KQLR” disclosed as SEQ ID NO: 21) in crystal form. The diffraction data are used to calculate an electron density map of the repeating unit of the crystal. The electron density maps are then used to establish the positions of the individual atoms of the HGFA, HGFA:Fab40, HGFA:Fab40. ΔTrp, or HGFA:KQLR:Fab40. ΔTrp complexes (“KQLR” disclosed as SEQ ID NO: 21).

Slight variations in structure coordinates can be generated by mathematically manipulating the HGFA, HGFA:Fab40, HGFA:Fab40. ΔTrp, or HGFA:KQLR:Fab40. ΔTrp complex (“KQLR” disclosed as SEQ ID NO: 21) structure coordinates. For example, the structure coordinates as set forth in Tables 7, 8 and/or 9 could be manipulated by crystallographic permutations of the structure coordinates, fractionalization of the structure coordinates, integer additions or subtractions to sets of the structure coordinates, inversion of the structure coordinates, or any combination of the above. Alternatively, modifications in the crystal structure due to mutations, additions, substitutions, deletions, and combinations thereof, of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the HGFA, HGFA:Fab40, HGFA:Fab40. ΔTrp, or HGFA:KQLR:Fab40. ΔTrp (“KQLR” disclosed as SEQ ID NO: 21) would not be expected to significantly alter the nature of chemical entities such as ligands that could associate with a binding site or other structural features of HGFA. In this context, the phrase “associating with” refers to a condition of proximity between a ligand, or portions thereof, and a HGFA molecule or portions thereof. The association may be non-covalent, wherein the juxtaposition is energetically favored by hydrogen bonding, van der Waals forces, and/or electrostatic interactions, or it may be covalent.

HGFA residues that form a binding site for an allosteric inhibitor of HGFA are described in the present application. The identification of a distinct binding site for an allosteric modulator on HGFA can be used to design new classes of HGFA modulators, such as antagonists, agonists, and like agents, having therapeutic applications, such as, for treating cancer.

### Structure of the HGFA/Fab40 Complex

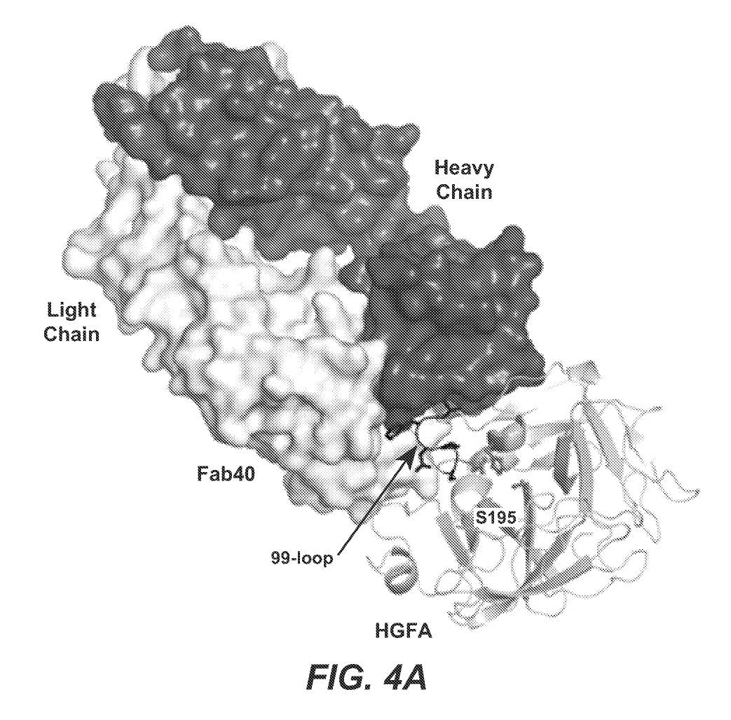

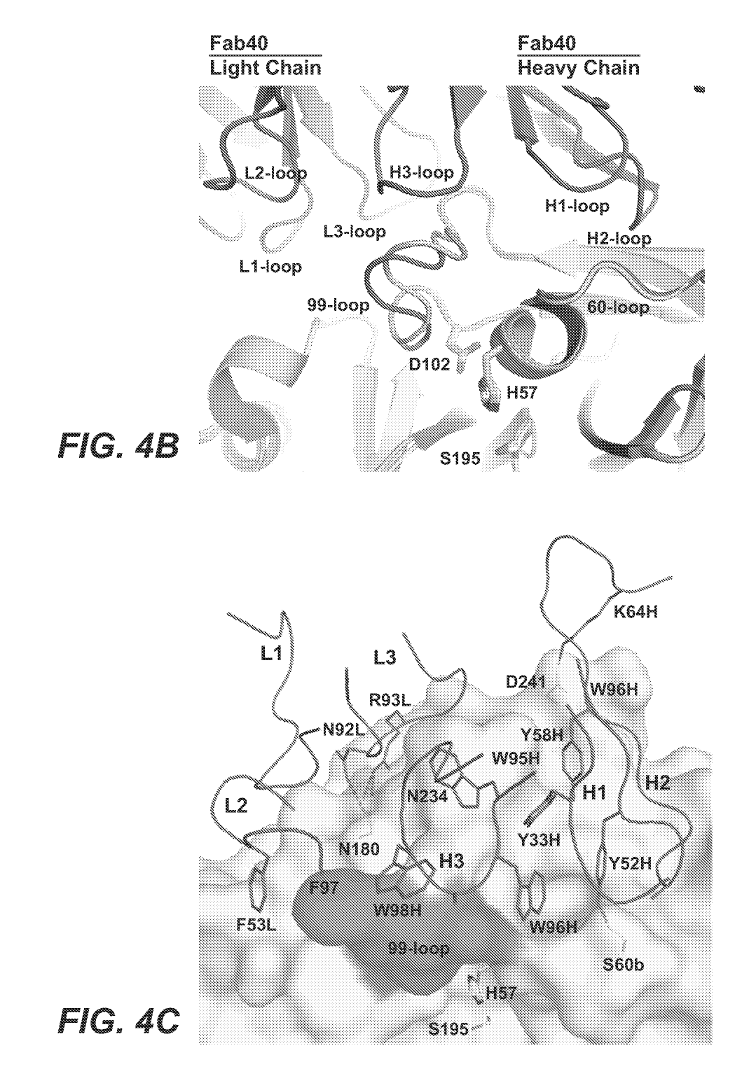

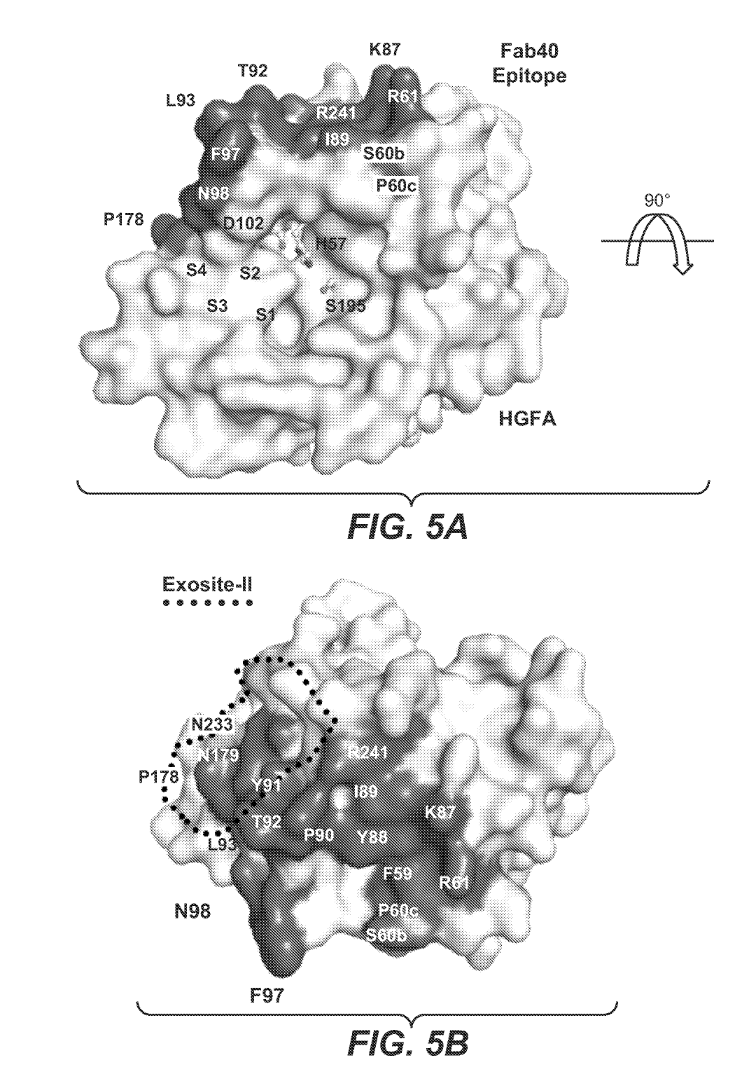

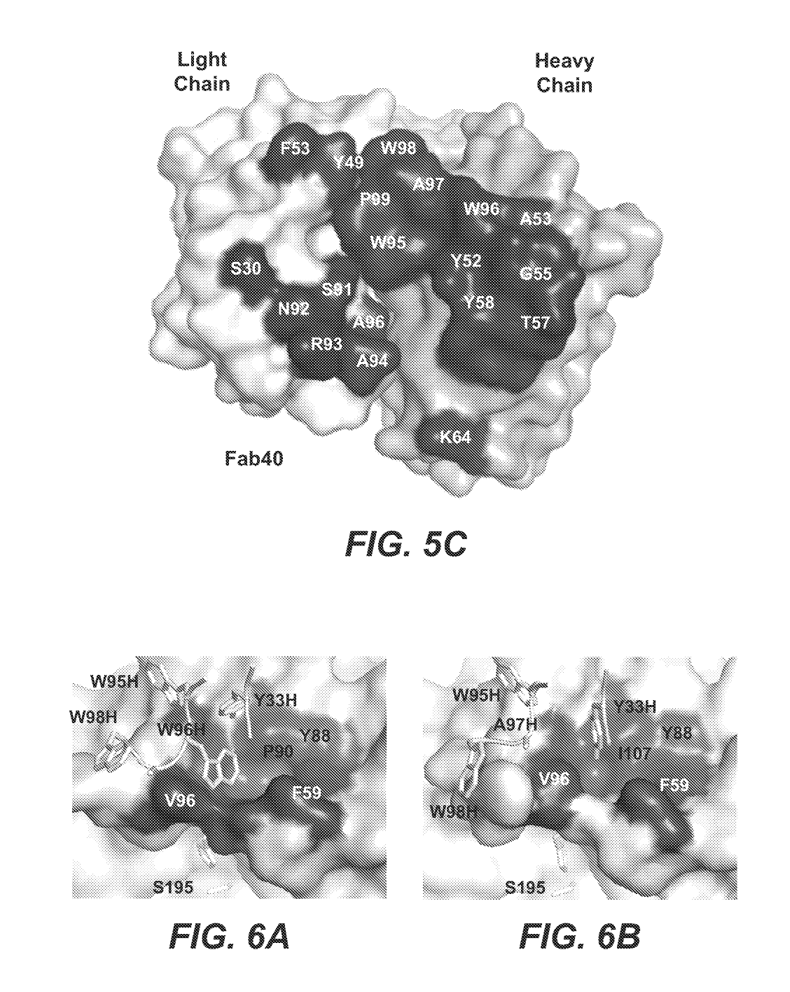

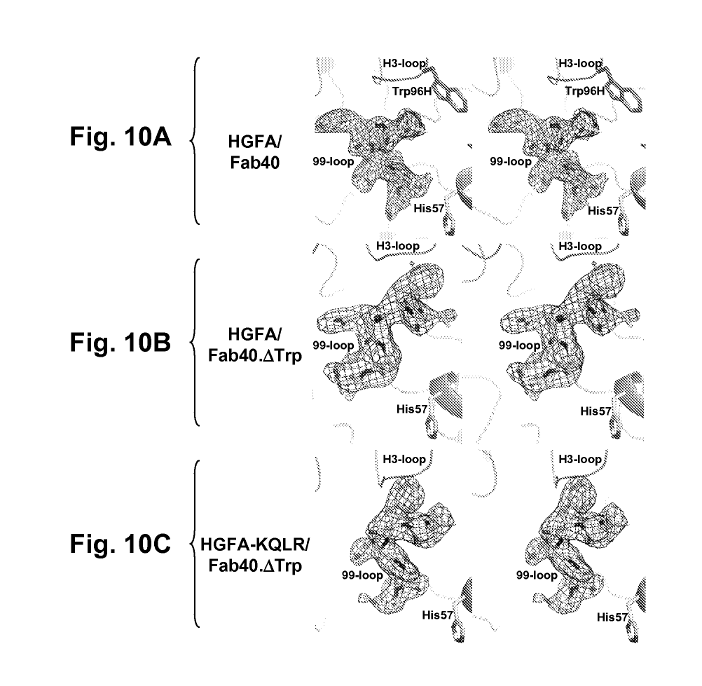

The 2.35 Å resolution structure of the HGFA/Fab40 complex shows that Fab40 uses all six CDR loops to bind to a flat epitope at the periphery of the substrate binding cleft, encompassing the 60-loop (e.g., native number 451-454; chymotrypsin numbers 60A-D) and 99-loop (e.g., native numbers 490-493; chymotrypsin numbers 96, 97, 98, and 99a) (FIG. 4a-b). The conformation of the catalytic triad (His-57, Asp-102 and Ser-195) has no significant changes compared to other known structures of HGFA (FIG. 4b) and the substrate subsites S1-S4 are unoccupied (FIG. 5a). The closest atom of Fab40 is >15 Å from the active site Ser195 residue indicating an allosteric mode of inhibition. The key feature of the HGFA/Fab40 complex is a large conformational change in the 99-loop (FIG. 4b and FIG. 10, illustrating the quality of the electron density map). No other significant changes in the HGFA structure are observed suggesting that this is the reason for antibody induced inhibition. The ‘allosteric switch’, embodied in the conformation of the 99-loop, is evident from the comparison of the HGFA/Fab40 structure with other structures of HGFA (Shia et al, 2005; Wu et al, 2007), in which the 99-loop is in a ‘competent’ conformation (catalytically active form of HGFA) state. The Phe97 of the 99-loop in the new conformation (‘non-competent’ conformation, catalytically inactive form of HGFA) is buried in a hydrophobic groove formed at the interface of light chain (Tyr49L, Ser50L and Phe53L) and heavy chain (Trp98H and Pro99H) of Fab40 (FIG. 4c). The epitope is centered on Leu-93 of the protruding 99-loop (FIG. 5a,b), which is sandwiched in a cleft between the CDR loops L3 and H3. The 99-loop and 60-loop are involved in intimate contacts mostly with heavy chain CDR residues. The heavy and light chains contribute 65% and 35% of buried surface area to the complex, respectively (FIG. 5c). The total solvent-accessible surface area of HGFA buried upon Fab40 binding is ˜1030 Å². Altogether, 18 hydrogen bonds and one electrostatic interaction (Asp241-Lys64H) are formed between HGFA and Fab40 (Table 3). Several hydrophobic residues like Trp95H, Trp96H, Trp98H and Tyr33H, Tyr52H, Tyr58H bind into small pockets at the back side of the 60- and 99-loops (FIG. 4c). The Fab40 epitope on HGFA has significant overlap with a region corresponding to exosite II in thrombin, an electropositive region that interacts with thrombin regulators (FIG. 5b). However, unlike exosite II interactions in coagulation proteases which are primarily electrostatic in nature (Bock et al, 2007), the binding of Fab40 to HGFA involves mainly hydrogen bonding and van der Waals interactions.

Structure of the HGFA/Fab40.ΔTrp complex

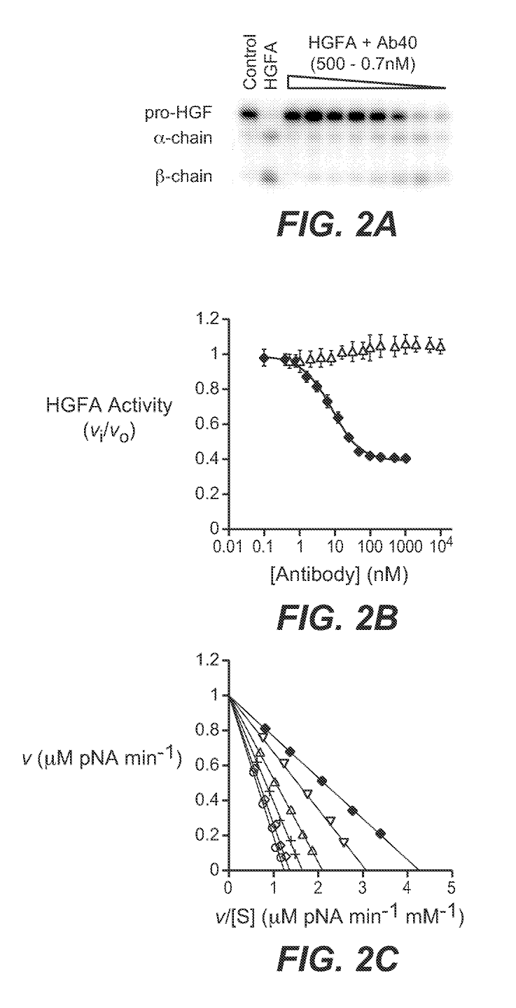

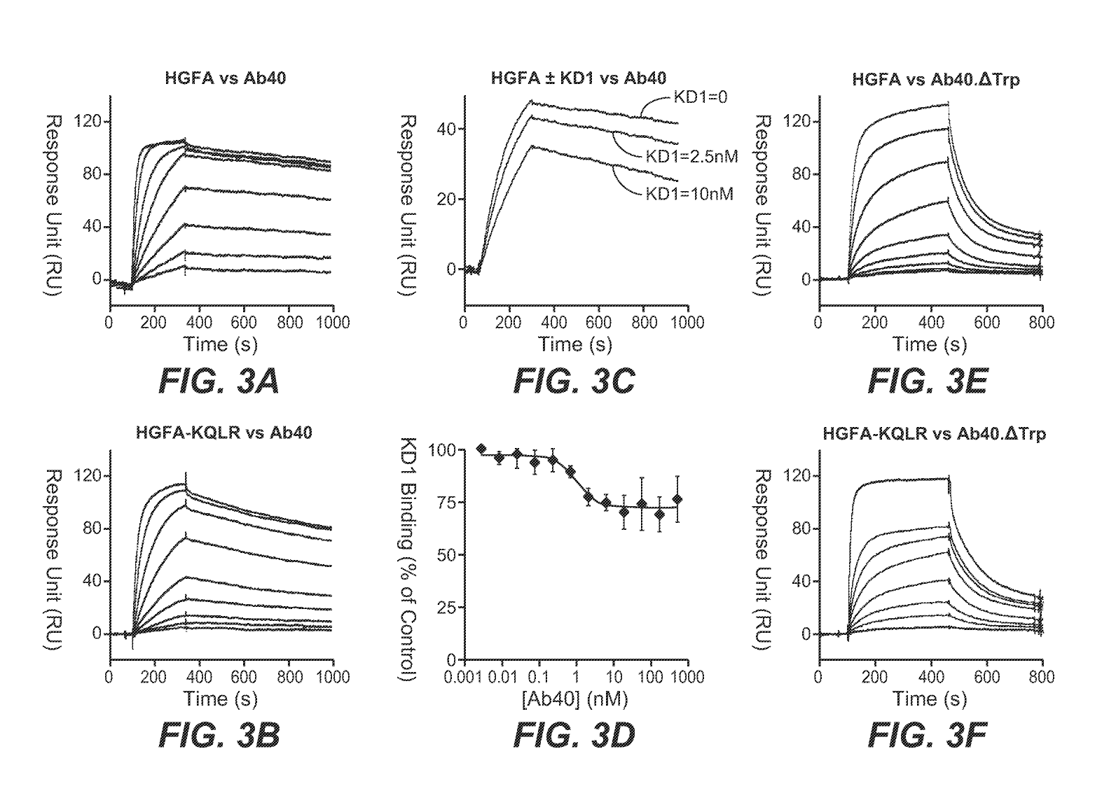

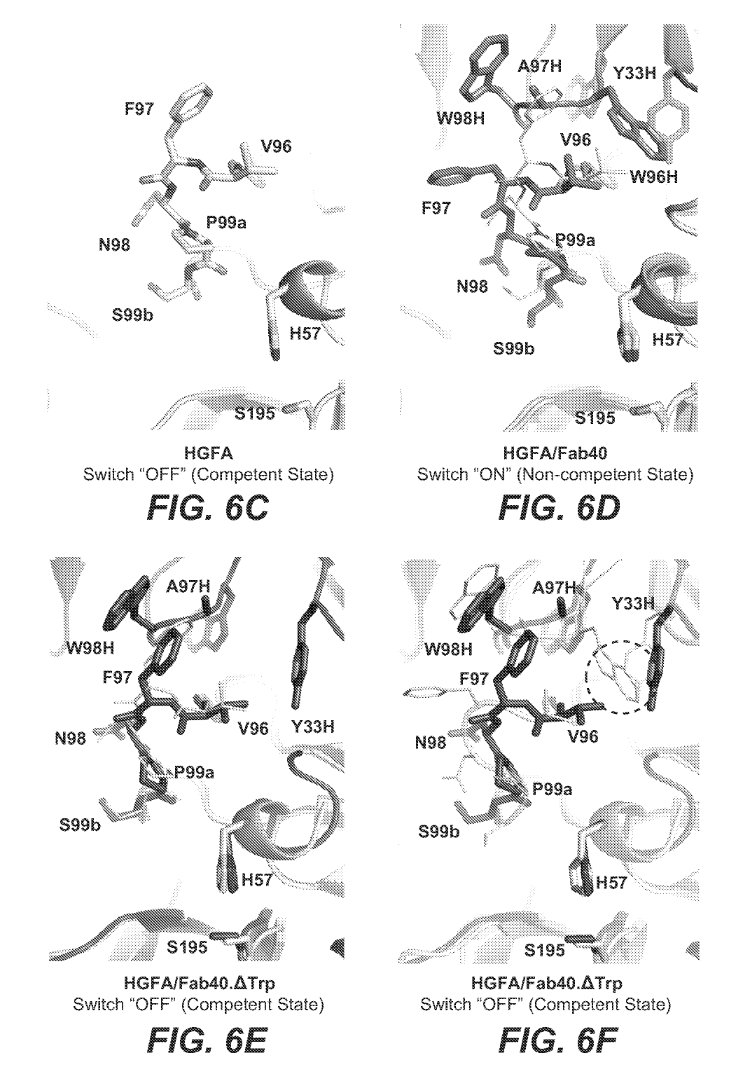

The overall structure of HGFA/Fab40.ΔTrp (2.90 Å) is very similar to that of HGFA/Fab40. The changes are very minimal and confined to residues Ala97H and Trp98H in the CDR-H3 loop (FIG. 6e-f). The side chain of Tyr33H flips around its χ₁ torsion angle and partly fills the deep hydrophobic pocket which was occupied by Trp96H in the HGFA/Fab40 structure (FIG. 6b). The size of this hydrophobic pocket is now reduced due to the movement of residues lining this pocket, principally Ser60, Pro90 and Tyr94 of HGFA. Remarkably the 99-loop reverted to the ‘competent’ state, as observed in other structures of HGFA (FIG. 6e-f). Ab40.ΔTrp was no longer an inhibitor of HGFA as determined by enzymatic assays (FIG. 2b). It was striking that such a subtle change was enough to remove the inhibitory activity while retaining binding, albeit with much reduced affinity (Table 1). Moreover, unlike Ab40, presence of the KQLR (SEQ ID NO: 21) inhibitor in the HGFA active site did not affect Ab40.ΔTrp binding as indicated by the similar K_{D} values for either HGFA or HGFA-KQLR complex (“KQLR” disclosed as SEQ ID NO: 21) (FIG. 3e,f). Thus, the data support our hypothesis that the mechanism of allosteric inhibitory activity by Ab40 is primarily driven by a significant change of the 99-loop conformation.

### Structure of the HGFA/KQLR/Fab40.ΔTrp Complex (“KQLR” Disclosed as SEQ ID NO: 21)

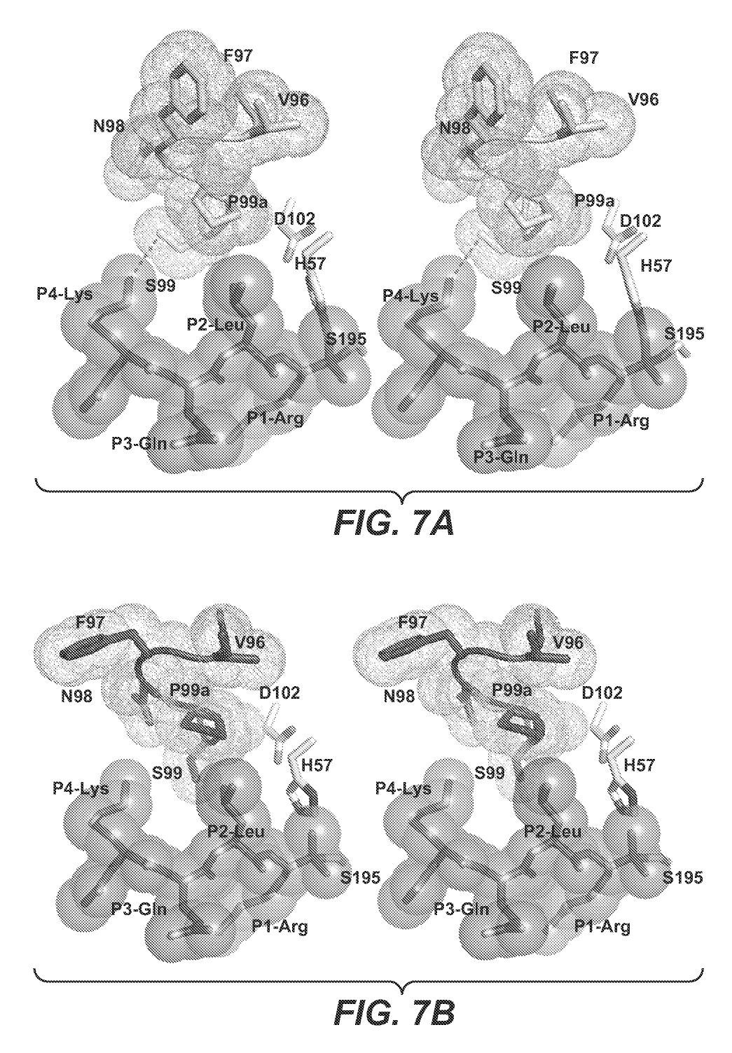

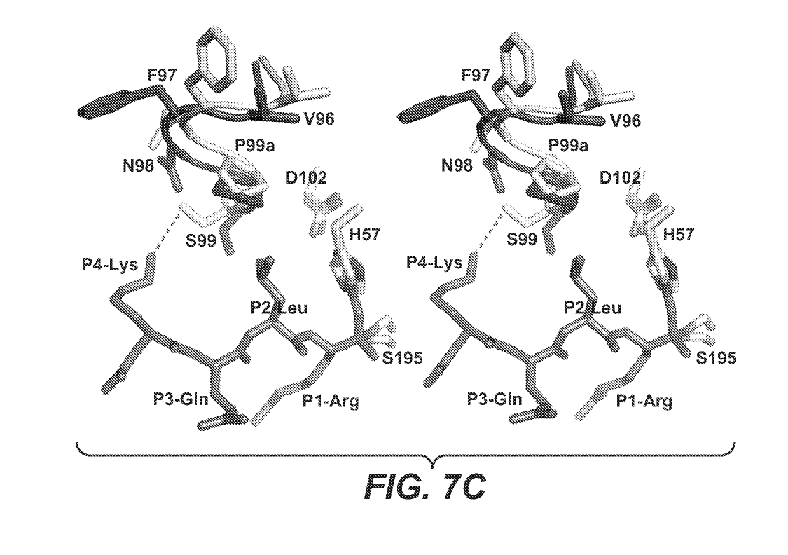

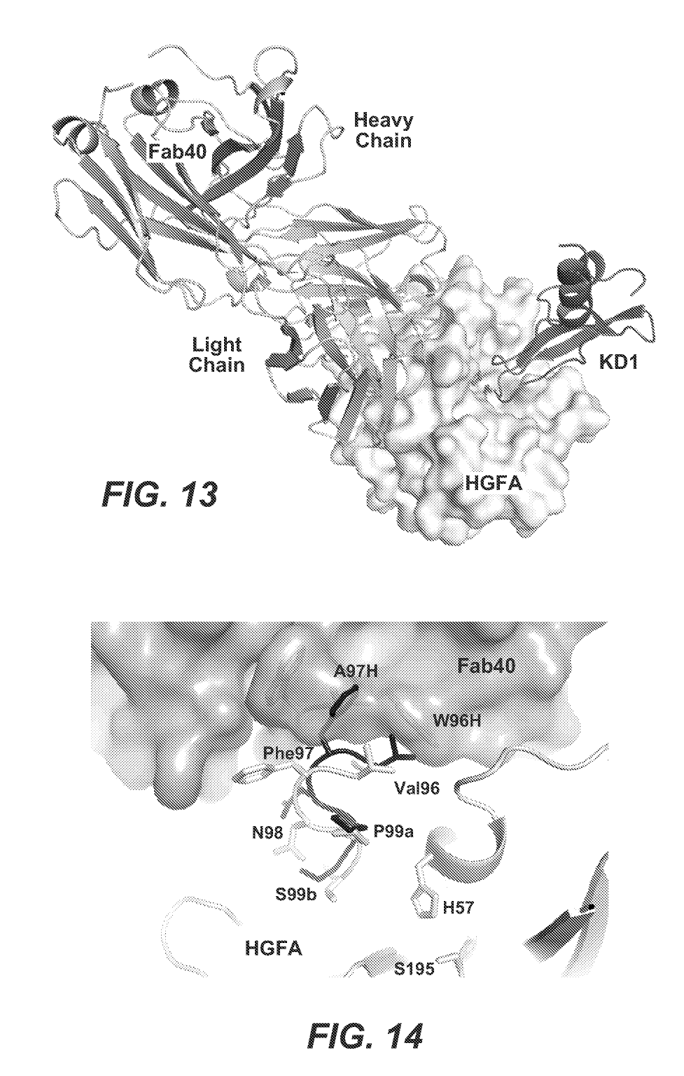

The structure of the protease domain of HGFA in HGFA-KQLR/Fab40.ΔTrp complex (“KQLR” disclosed as SEQ ID NO: 21) is very similar to HGFA/Fab40.ΔTrp complex with rmsd (for all atoms) of 0.39 Å(FIG. 12). The conformation of the 99-loop in HGFA-KQLR/Fab40.ΔTrp (“KQLR” disclosed as SEQ ID NO: 21) structure is in the ‘competent’ state as found in HGFA/Fab40.ΔTrp and other structures of HGFA. Thus, the HGFA-KQLR/Fab40.ΔTrp (“KQLR” disclosed as SEQ ID NO: 21) is a good alternative in the absence of a HGFA-KQLR (“KQLR” disclosed as SEQ ID NO: 21) structure, to define the substrate binding subsites in HGFA. Superposition of HGFA/Fab40 structure with HGFA-KQLR/Fab40.ΔTrp (“KQLR” disclosed as SEQ ID NO: 21) revealed a plausible cause for the allosteric inhibition. The movement of the 99-loop lead to a partial collapse of the subsite S2 and the reorganization of subsite S4, perturbing the interactions with substrate residues P2 and P4. First, the movement of the 99-loop residues Pro99a and Ser99, which are part of the S2 pocket, positions them too close to the P2-Leu (FIG. 7b), creating a steric clash. Secondly, the repositioned hydroxyl side chain of the S4 residue Ser99 can no longer form the hydrogen bond with P4-Lys (FIG. 7a,b) and is positioned too close to P2-Leu. Furthermore, the movement of the S2 pocket residue Pro99a also plays a key role in defining the allosteric nature of the inhibition of KD1 binding to HGFA by Ab40. Superposition of HGFA/Fab40 and HGFA/KD1 (Shia et al, 2005) complexes showed that there was no overlap between the KD1- and Fab40 epitopes (FIG. 13). A model of HGFA/Fab40/KD1 predicts a possibility of Pro99a to sterically clash with Cys38 and Leu39 of KD1 at the S2 subsite.

The structure of the protease domain of HGFA in HGFA-KQLR/Fab40.ΔTrp complex (“KQLR” disclosed as SEQ ID NO: 21) is very similar to HGFA/Fab40.ΔTrp complex with rmsd (for all atoms) of 0.39 Å (FIG. 12). The conformation of the 99-loop in HGFA-KQLR/Fab40.ΔTrp (“KQLR” disclosed as SEQ ID NO: 21) structure is in the ‘competent’ state as found in HGFA/Fab40.ΔTrp and other structures of HGFA. Thus, the HGFA-KQLR/Fab40.ΔTrp (“KQLR” disclosed as SEQ ID NO: 21) is a good alternative in the absence of a HGFA-KQLR (“KQLR” disclosed as SEQ ID NO: 21) structure, to define the substrate binding subsites in HGFA. Superposition of HGFA/Fab40 structure with HGFA-KQLR/Fab40.ΔTrp (“KQLR” disclosed as SEQ ID NO: 21) revealed a plausible cause for the allosteric inhibition. The movement of the 99-loop lead to a partial collapse of the subsite S2 and the reorganization of subsite S4, perturbing the interactions with substrate residues P2 and P4. First, the movement of the 99-loop residues Pro99a and Ser99, which are part of the S2 pocket, positions them too close to the P2-Leu (FIG. 7b), creating a steric clash. Secondly, the repositioned hydroxyl side chain of the S4 residue Ser99 can no longer form the hydrogen bond with P4-Lys (FIG. 7a,b) and is positioned too close to P2-Leu. Furthermore, the movement of the S2 pocket residue Pro99a also plays a key role in defining the allosteric nature of the inhibition of KD1 binding to HGFA by Ab40. Superposition of HGFA/Fab40 and HGFA/KD1 (Shia et al, 2005) complexes showed that there was no overlap between the KD1- and Fab40 epitopes (FIG. 13). A model of HGFA/Fab40/KD1 predicts a possibility of Pro99a to sterically clash with Cys38 and Leu39 of KD1 at the S2 subsite.

### 3. Structurally Equivalent Crystal Structures

Various computational analyses can be used to determine whether a molecule or portions of the molecule defining structure features are “structurally equivalent,” defined in terms of its three-dimensional structure, to all or part of an activated unbound HGFA or HGFA bound to an allosteric inhibitor, such as a Fab40 antibody, or HGFA present in HGFA:Fab40. ΔTrp or HGFA:KQLR:Fab40. ΔTrp complexes (“KQLR” disclosed as SEQ ID NO: 21). Such analyses may be carried out in current software applications, such as the Molecular Similarity application of QUANTA (Molecular Simulations Inc., San Diego, Calif.), Version 4.1, and as described in the accompanying User's Guide.

The Molecular Similarity application permits comparisons between different structures, different conformations of the same structure, and different parts of the same structure. A procedure used in Molecular Similarity to compare structures comprises: 1) loading the structures to be compared; 2) defining the atom equivalences in these structures; 3) performing a fitting operation; and 4) analyzing the results.

One structure is identified as the target (i.e., the fixed structure); all remaining structures are working structures (i.e., moving structures). Since atom equivalency within QUANTA is defined by user input, for the purpose of this disclosure equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in Angstroms, is reported by QUANTA.

Structurally equivalent crystal structures have portions of the two molecules that are substantially identical, within an acceptable margin of error. The margin of error can be calculated by methods known to those of skill in the art. In some embodiments, any molecule or molecular complex or any portion thereof, that has a root mean square deviation of conserved residue backbone atoms (N, Cα, C, O) of less than about 0.70 Å, preferably 0.5 Å. For example, structurally equivalent molecules or molecular complexes are those that are defined by the entire set of structure coordinates listed in Table 7 or 8 ±a root mean square deviation from the conserved backbone atoms of those amino acids of not more than 0.70 Å, preferably 0.5 Å. The term “root mean square deviation” means the square root of the arithmetic mean of the squares of the deviations. It is a way to express the deviation or variation from a trend or object. For purposes of this disclosure, the “root mean square deviation” defines the variation in the backbone of a protein from the backbone of HGFA complex (as defined by the structure coordinates of the complex as described herein) or a defining structural feature thereof.

### 4. Structurally Homologous Molecules, Molecular Complexes, and Crystal Structures

Structure coordinates can be used to aid in obtaining structural information about another crystallized molecule or molecular complex. The method of the disclosure allows determination of at least a portion of the three-dimensional structure of molecules or molecular complexes that contain one or more structural features that are similar to structural features of at least a portion of the HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21). These molecules are referred to herein as “structurally homologous” to HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21). Similar structural features can include, for example, regions of amino acid identity, conserved active site or binding site motifs, and similarly arranged secondary structural elements (for example, binding sites for KD1 or HGF on HGFA; EGF-like domains; kringle domain; trypsin like serine protease domain; type I fibronectin domain; type II fibronectin domain; binding sites on KD1 or KQLR (SEQ ID NO: 21) for HGFA; and Kunitz domains on the inhibitor).

Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Two amino acid sequences are compared using the BLAST program, version 2.0.9, of the BLAST 2 search algorithm, as described by Tatusova et al. (56), and available at http:www.ncbi.nlm.nih.gov/BLAST/. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.” In some embodiments, a structurally homologous molecule is a protein that has an amino acid sequence having at least 80% identity with a wild type or recombinant amino acid sequence of HGFA, in some embodiments, a portion of HGFA comprising the amino acid sequence shown in Table 6. More preferably, a protein that is structurally homologous to HGFA includes at least one contiguous stretch of at least 50 amino acids that has at least 80% amino acid sequence identity with the analogous portion of the wild type or recombinant HGFA. Methods for generating structural information about the structurally homologous molecule or molecular complex are well known and include, for example, molecular replacement techniques.

Therefore, in another embodiment this disclosure provides a method of utilizing molecular replacement to obtain structural information about a molecule or molecular complex whose structure is unknown comprising:

(a) generating an X-ray diffraction pattern from a crystallized molecule or molecular complex of unknown or incompletely known structure; and

(b) applying at least a portion of the structural coordinates of HGFA complex to the X-ray diffraction pattern to generate a three-dimensional electron density map of the molecule or molecular complex whose structure is unknown or incompletely known.

By using molecular replacement, all or part of the structure coordinates of HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) as provided by this disclosure can be used to determine the unsolved structure of a crystallized molecule or molecular complex more quickly and efficiently than attempting to determine such information ab initio. Coordinates of structural features of HGFA can be utilized including that of trypsin-like serine protease domain.

Molecular replacement can provide an accurate estimation of the phases for an unknown or incompletely known structure. Phases are one factor in equations that are used to solve crystal structures, and this factor cannot be determined directly. Obtaining accurate values for the phases, by methods other than molecular replacement, can be a time-consuming process that involves iterative cycles of approximations and refinements and greatly hinders the solution of crystal structures. However, when the crystal structure of a protein containing at least a structurally homologous portion has been solved, molecular replacement using the known structure provide a useful estimate of the phases for the unknown or incompletely known structure.

Thus, this method involves generating a preliminary model of a molecule or molecular complex whose structure coordinates are unknown, by orienting and positioning the relevant portion of the HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) within the unit cell of the crystal of the unknown molecule or molecular complex. This orientation or positioning is conducted so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure. This map, in turn, can be subjected to established and well-known model building and structure refinement techniques to provide a final, accurate structure of the unknown crystallized molecule or molecular complex (see for example, Lattman, 1985. Methods in Enzymology 115:55-77).

Structural information about a portion of any crystallized molecule or molecular complex that is sufficiently structurally homologous to a portion of HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) can be solved by this method. In addition to a molecule that shares one or more structural features with the HGFA, such as the trypsin like serine protease domain, and/or Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) as described above, a molecule that has similar bioactivity, such as the same catalytic activity, substrate specificity or ligand binding activity as HGFA, KD1 and/or HGFA:KD1, may also be sufficiently structurally homologous to a portion of the HGFA, a portion of KD1, and/or HGFA:KD1 to permit use of the structure coordinates of HGFA:KD1 to solve its crystal structure or identify structural features that are similar to those identified in the HGFA described herein. It will be appreciated that amino acid residues in the structurally homologous molecule identified as corresponding to the HGFA structural feature may have different amino acid numbering.

In one embodiment of the disclosure, the method of molecular replacement is utilized to obtain structural information about a molecule or molecular complex, wherein the molecule or molecular complex includes at least one HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) subunit or homolog. In the context of the present disclosure, a “structural homolog” of the HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) is a protein that contains one or more amino acid substitutions, deletions, additions, or rearrangements with respect to the amino acid sequence of HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) but that, when folded into its native conformation, exhibits or is reasonably expected to exhibit at least a portion of the tertiary (three-dimensional) structure of at least a portion of HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21). A portion of the HGFA includes the binding site for an allosteric inhibitor.

A heavy atom derivative of HGFA is also included as a HGFA homolog. The term “heavy atom derivative” refers to derivatives of HGFA:Fab40, HGFA:Fab40. ΔTrp, or HGFA:KQLR:Fab40. ΔTrp complex (“KQLR” disclosed as SEQ ID NO: 21) produced by chemically modifying a crystal of HGFA or both. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the protein (Blundell, et al., 1976, Protein Crystallography, Academic Press, San Diego, Calif.).

Variants may be prepared, for example, by expression of HGFA cDNA previously altered in its coding sequence by oligonucleotide-directed mutagenesis as described herein. Variants may also be generated by site-specific incorporation of unnatural amino acids into HGFA proteins using known biosynthetic methods (Noren, et al., 1989, Science 244:182-88). In this method, the codon encoding the amino acid of interest in wild-type HGFA is replaced by a “blank” nonsense codon, TAG; using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant HGFA with the site-specific incorporated unnatural amino acid.

For example, structurally homologous molecules can contain deletions or additions of one or more contiguous or noncontiguous amino acids, such as a loop or a domain. Structurally homologous molecules also include “modified” HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and like modifications. It will be appreciated that amino acid residues in the structurally homologous molecule identified as corresponding to activated HGFA or other structural feature of the HGFA may have different amino acid numbering.

The structure coordinates of HGFA are also particularly useful to solve or model the structure of crystals of HGFA, HGFA variants, Fab40, Fab40 variants, Fab40. ΔTrp, Fab40. ΔTrp variants, KQLR (SEQ ID NO: 21), KQLR (SEQ ID NO: 21) variants, HGFA homologs, Fab40 homologs, Fab40. ΔTrp homologs, KQLR (SEQ ID NO: 21) homologs which are co-complexed with a variety of ligands (e.g., a ligand binding the allosteric binding site). This approach enables the determination of the optimal sites for interaction between ligand, including candidate HGFA ligands. Potential sites for modification within the various binding sites (such as an HGFA allosteric binding site) of the molecule can also be identified. This information provides an additional tool for determining more efficient binding interactions, for example, increased hydrophobic or polar interactions, between HGFA and a ligand. For example, high-resolution X-ray diffraction data collected from crystals exposed to different types of solvent allows the determination of where each type of solvent molecule resides. Small molecules that bind tightly to those sites can then be designed and synthesized and tested for their HGFA affinity, and/or inhibition activity.

All of the complexes referred to above may be studied using well-known X-ray diffraction techniques and may be refined versus 1.5-3.5 Å resolution X-ray data to an R-factor of about 0.30 or less using computer software, such as X-PLOR (Yale University, distributed by Molecular Simulations, Inc.)(see for example, Blundell, et al. 1976. Protein Crystallography, Academic Press, San Diego, Calif., and Methods in Enzymology, Vol. 114 & 115, H. W. Wyckoff et al., eds., Academic Press (1985)). This information may thus be used to optimize known HGFA modulators, and more importantly, to design new HGFA modulators.

The disclosure also includes the unique three-dimensional configuration defined by a set of points defined by the structure coordinates for a molecule or molecular complex structurally homologous to HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21) as determined using the method of the present disclosure, structurally equivalent configurations, and magnetic storage media including such set of structure coordinates.

### 5. Homology Modeling

Using homology modeling, a computer model of a homolog, eg, an HGFA homolog, can be built or refined without crystallizing the homolog. First, a preliminary model of the homolog is created by sequence alignment with HGFA, Fab40, Fab40. ΔTrp, KQLR (SEQ ID NO: 21) or a HGFA:Fab40, HGFA:Fab40. ΔTrp, or HGFA:KQLR:Fab40. ΔTrp complex (“KQLR” disclosed as SEQ ID NO: 21), secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. If the homolog has been crystallized, the final homology model can be used to solve the crystal structure of the homolog by molecular replacement, as described above. Next, the preliminary model is subjected to energy minimization to yield an energy-minimized model. The energy-minimized model may contain regions where stereochemistry restraints are violated, in which case such regions are remodeled to obtain a final homology model. The homology model is positioned according to the results of molecular replacement, and subjected to further refinement including molecular dynamics calculations.

### 6. Methods for Identification of Modulators of HGFA

Potent and selective ligands that modulate activity (antagonists and agonists) can be identified using the three-dimensional model of the HGFA using the coordinates of Table 7, 8 and/or 9. In some embodiments, the three-dimensional model of the allosteric binding site on HGFA and/or other structural features are produced using the coordinates of Table 7. Using this model, candidate ligands that interact with the HGFA, e.g., the HGFA allosteric binding site, are assessed for the desired characteristics (e.g., interaction with HGFA) by fitting against the model, and the result of the interactions is predicted. Agents predicted to be molecules capable of modulating the activity of HGFA can then be further screened or confirmed against known bioassays. For example, the ability of an agent to inhibit the morphogenic or mitogenic effects of HGF can be measured using assays known in the art. Using the modeling information and the assays described, one can identify agents that possess HGFA-modulating properties. Modulators of HGFA of the present disclosure can include compounds or agents having, for example, allosteric regulatory activity.

Ligands which can interact with HGFA can also be identified using commercially available modeling software, such as docking programs, in which the solved crystal structure coordinates of Table 7, 8 and/or 9 can be computationally represented and screened against a large virtual library of small molecules or virtual fragment molecules for interaction with a site of interest, such as the HGFA allosteric binding site. Preferred small molecules or fragments identified in this way can be synthesized and contacted with the HGFA. The resulting molecular complex or association can be further analyzed by, for example, NMR or X-ray co-crystallography, to optimize the HGFA-ligand interaction and/or desired biological activity. Fragment-based drug discovery methods are known and computational tools for their use are commercially available, for example “SAR by NMR” (Shukers, S. B., et al., Science, 1996, 274, 1531-1534), “Fragments of Active Structures” (www.stromix.com; Nienaber, V. L., et al., Nat. Biotechnol., 2000, 18, 1105-1108), and “Dynamic Combinatorial X-ray Crystallography” (e.g., permitting self-selection by the protein molecule of self-assembling fragments; Congreve, M. S., et al., Angew. Chem., Int. Ed., 2003, 42, 4479-4482). Still other molecular modeling, and like methods are discussed below and in the Examples.

The present disclosure also includes identification of allosteric modulators of HGFA. Generally, these molecules can be identified using the three-dimensional model of HGFA using the coordinates of Table 7.

In another embodiment, a candidate modulator can be identified using a biological assay such as binding to HGFA, modulation (e.g., inhibition) of HGFA activation of HGF, modulation (e.g., inhibition) of HGF biological activity (such as modulating Met phosphorylation or modulating HGF β induced cell migration). The candidate modulator can then serve as a model to design similar agents and/or to modify the candidate modulator for example, to improve characteristics such as binding to HGFA. Design or modification of candidate modulators can be accomplished using the crystal structure coordinates and available software.

Binding Site and Other Structural Features

Applicants' disclosure provides information inter alia about the shape and structure of an allosteric binding site of HGFA in the presence of an allosteric inhibitor (Fab40 anti-HGFA antibody). The binding site is disclosed in an HGFA that is catalytically active (Table 8, 9) and a HGFA that is catalytically inactive (table 7). Binding sites are of significant utility in fields such as drug discovery. The association of natural ligands or substrates with the binding sites of their corresponding receptors or enzymes is the basis of many biological mechanisms of action. Similarly, many drugs exert their biological effects through association with the binding sites of receptors and enzymes. Such associations may occur with all or any part of the binding site. An understanding of such associations helps lead to the design of drugs having more favorable associations with their target, and thus improved biological effects. Therefore, this information is valuable in designing potential modulators of HGFA binding sites, as discussed in more detail below.

The amino acid constituents of a HGFA binding site as defined herein are positioned in three dimensions. The structural coordinates of HGFA with a bound allosteric inhibitor are in Table 7. The structural coordinates of HGFA bound to an antibody that is not an allosteric inhibitor are in Table 8 and 9. Generally, but not exclusively, methods of the invention (such as screening methods of the invention) use the structural coordinates of HGFA with a bound allosteric inhibitor, eg, as shown in Table 7. In one aspect, the structure coordinates defining a binding site of HGFA include structure coordinates of all atoms in the constituent amino acids; in another aspect, the structure coordinates of a binding site include structure coordinates of just the backbone atoms of the constituent atoms. HGFA that is bound to an allosteric inhibitor has a different conformation than when inhibitor is not bound. In the bound state, a number of amino acid residues form a pocket.

The term “HGFA allosteric binding site” includes all or a portion of a molecule or molecular complex whose shape is sufficiently similar to at least a portion of a binding site on HGFA for an allosteric inhibitor as to be expected to other allosteric inhibitors. A structurally equivalent ligand binding site is defined by a root mean square deviation from the structure coordinates of the backbone atoms of the amino acids that make up binding sites in HGFA for anti-HGFA antibody Ab40 of at most about 0.70 Å, preferably about 5 Å. In some embodiments, an allosteric binding site for an allosteric inhibitor of HGFA comprises, consists essentially of, or consists of at least one amino acid residue corresponding to residues 446, 449, 450, 452, 453, 455, 480, 481, 482, 483, 484, 485, 486, 487, 488, 489, 490, 491, 496, 499, 501, 578, 579, 580, 636, 637, 640, 643, 644, or mixtures thereof. In some embodiments, an allosteric binding site for the HGFA for an allosteric inhibitor of HGFA comprises, consists essentially of, or consists of at least one amino acid residue corresponding to residues 449, 450, 452, 482, 484, 485, 486, 487, 488, 489, 490, 491, or mixtures thereof. In some embodiments, an allosteric binding site for the HGFA for an allosteric inhibitor of HGFA comprises, consists essentially of, or consists of at least one amino acid residue corresponding to residues 446, 482, 484, 490, 499, 501, or mixtures thereof.

The HGFA allosteric binding site may be defined by those amino acids whose backbone atoms are situated within about 5 Å of one or more constituent atoms of a bound substrate or ligand. In yet another alternative, the HGFA allosteric binding site can be defined by those amino acids whose backbone atoms are situated within a sphere centered on the coordinates representing the alpha carbon atom of amino acid V490 on the HGFA, the sphere having a radius of about 5-6 Å, for example 5.8 Å.

Rational Drug Design

Computational techniques can be used to screen, identify, select, design ligands, and combinations thereof, capable of associating with HGFA or structurally homologous molecules. Candidate modulators of HGFA may be identified using functional assays, such as binding to HGFA, and novel modulators designed based on the structure of the candidate molecules so identified. Knowledge of the structure coordinates for HGFA permits, for example, the design, the identification of synthetic compounds, and like processes, and the design, the identification of other molecules and like processes, that have a shape complementary to the conformation of the HGFA binding sites. In particular, computational techniques can be used to identify or design ligands, such as agonists and/or antagonists, that associate with a HGFA binding site. Antagonists may bind to or interfere with all or a portion of an active site of HGFA, and can be competitive, non-competitive, or uncompetitive inhibitors. Once identified and screened for biological activity, these agonists, antagonists, and combinations thereof, may be used therapeutically and/or prophylactically, for example, to block HGFA activity and thus prevent the onset and/or further progression of diseases associated with HGFA activity. Structure-activity data for analogues of ligands that bind to or interfere with HGFA binding sites can also be obtained computationally.

In some embodiments, agonists or antagonists can be designed to include components that preserve and/or strengthen the interactions. Such antagonists or agonists would include components that are able to interact, for example, hydrogen bond with the charged amino acids found in, e.g., either an allosteric binding site of HGFA (activated or unactivated, bound to substrate or unbound to substrate) or HGFA bound to an inhibitor or both.

In some embodiments, for HGFA, antagonist or agonist molecules are designed or selected that can interact with at least one or all amino acid residues that comprise, consist essentially of, or consist of at least one amino acid residue corresponding to an amino acid residue in one or more of the allosteric binding site, or mixtures thereof.

In some embodiments, an inhibitor will be designed to interact with an amino acid at least one or all residues in the S1 subsite comprising, consisting essentially of, or consisting of amino acid residues of HGFA: Asp592(189), Ala593(190), Cys594(191), Gln595(192), Asp597(194), Ile616(213), Trp618(215), Gly619(216), Ser620(217), Gly621(219), Cys622(222), Tyr631(228), or mixtures thereof. In a specific embodiment, an inhibitor may be designed to interact with Trp618(215), Gly619(216) and Ser620(617) in the S1 subsite.

Comparison of the allosteric binding site on HGFA to analogous sites of related protease domains will direct design of inhibitors that favor HGFA over the related proteases. The crystal structures of other related proteases, if they are available can be utilized to maximize fit and/or interaction with HGFA allosteric binding site and minimize the fit and/or interactions with amino acids in the corresponding positions in other proteases.

Data stored in a machine-readable storage medium that is capable of displaying a graphical three-dimensional representation of the structure of HGFA or a structurally homologous molecule or molecular complex, as identified herein, or portions thereof may thus be advantageously used for drug discovery. The structure coordinates of the ligand are used to generate a three-dimensional image that can be computationally fit to the three-dimensional image of HGFA, Fab40, Fab40. ΔTrp, or KQLR (SEQ ID NO: 21), or a structurally homologous molecule. The three-dimensional molecular structure encoded by the data in the data storage medium can then be computationally evaluated for its ability to associate with ligands. When the molecular structures encoded by the data is displayed in a graphical three-dimensional representation on a computer screen, the protein structure can also be visually inspected for potential association with ligands.

One embodiment of the method of drug design involves evaluating the potential association of a candidate ligand with HGFA or a structurally homologous molecule or homologous complex, particularly with at least one amino acid residue in a binding site (e.g., an allosteric binding site) of the HGFA or a portion of the binding site. The method of drug design thus includes computationally evaluating the potential of a selected ligand to associate with any of the molecules or molecular complexes set forth above. This method includes the steps of: (a) employing computational means, for example, such as a programmable computer including the appropriate software known in the art or as disclosed herein, to perform a fitting operation between the selected ligand and a ligand binding site or a subsite of the ligand binding site of the molecule or molecular complex; and (b) analyzing the results of the fitting operation to quantify the association between the ligand and the ligand binding site. Optionally, the method further comprises analyzing the ability of the selected ligand to interact with amino acids in the HGFA binding site and/or subsite. The method may also further comprise optimizing the fit of the ligand for the binding site of HGFA as compared to other proteases. Optionally, the selected ligand can be synthesized, cocrystallized with HGFA, and further modifications to selected ligand can be made to enhance inhibitory activity or fit in the binding pocket. In addition as described previously, portions of Fab40 that bind to HGFA can be modified and utilized in the method described herein. Other structural features of the HGFA and/or HGFA:Fab40, HGFA:Fab40. ΔTrp, or HGFA:KQLR:Fab40. ΔTrp complex (“KQLR” disclosed as SEQ ID NO: 21) can also be analyzed in the same manner.

In another embodiment, the method of drug design involves computer-assisted design of ligand that associates with HGFA, its homologs, or portions thereof. Ligands can be designed in a step-wise fashion, one fragment at a time, or may be designed as a whole or de novo. Ligands can be designed based on the structure of molecules that can modulate at least one biological function of HGFA, such as Fab40 and other naturally occurring inhibitors of HGFA. In addition, the inhibitors can be modeled on other known inhibitors of serine proteases.

In some embodiments, to be a viable drug candidate, the ligand identified or designed according to the method must be capable of structurally associating with at least part of a HGFA binding site (e.g., a HGFA allosteric binding site), and must be able, sterically and energetically, to assume a conformation that allows it to associate with the HGFA binding site. Non-covalent molecular interactions important in this association include hydrogen bonding, van der Waals interactions, hydrophobic interactions, and/or electrostatic interactions. In some embodiments, an agent may contact at least one amino acid position in the HGFA binding site (e.g., an allosteric binding site) for an inhibitor, such as Fab40. Conformational considerations include the overall three-dimensional structure and orientation of the ligand in relation to the ligand binding site, and the spacing between various functional groups of a ligand that directly interact with the HGFA binding site or homologs thereof.

Optionally, the potential binding of a ligand to a HGFA binding site (e.g., an allosteric binding site) is analyzed using computer modeling techniques prior to the actual synthesis and testing of the ligand. If these computational experiments suggest insufficient interaction and association between it and the HGFA binding site, testing of the ligand is obviated. However, if computer modeling indicates a strong interaction, the molecule may then be synthesized and tested for its ability to bind to or interfere with a HGFA binding site. Binding assays to determine if a compound actually modulates HGFA activity can also be performed and are well known in the art.

Several methods can be used to screen ligands or fragments for the ability to associate with a HGFA binding site (e.g., an allosteric binding site). This process may begin by visual inspection of, for example, a HGFA binding site on the computer screen based on the HGFA structure coordinates or other coordinates which define a similar shape generated from the machine-readable storage medium. Selected ligands may then be positioned in a variety of orientations, or docked, within the binding site. Docking may be accomplished using software such as QUANTA and SYBYL, followed by energy minimization and molecular dynamics with standard molecular mechanics force fields, such as CHARMM and AMBER.

Specialized computer programs may also assist in the process of selecting ligands. Examples include GRID (Hubbard, S. 1999. Nature Struct. Biol. 6:711-4); MCSS (Miranker, et al. 1991. Proteins 11:29-34) available from Molecular Simulations, San Diego, Calif.; AUTODOCK (Goodsell, et al. 1990. Proteins 8:195-202) available from Scripps Research Institute, La Jolla, Calif.; and DOCK (Kuntz, et al. 1982. J. Mol. Biol. 161:269-88) available from University of California, San Francisco, Calif.

HGFA binding ligands can be designed to fit a HGFA binding site (e.g., an allosteric binding site), optionally as defined by the binding of a known modulator or one identified as modulating the activity of HGFA. There are many ligand design methods including, without limitation, LUDI (Bohm, 1992. J. Comput. Aided Molec. Design 6:61-78) available from Molecular Simulations Inc., San Diego, Calif.; LEGEND (Nishibata, Y., and Itai, A. 1993. J. Med. Chem. 36:2921-8) available from Molecular Simulations Inc., San Diego, Calif.; LeapFrog, available from Tripos Associates, St. Louis, Mo.; and SPROUT (Gillet, et al. 1993. J. Comput. Aided Mol. Design. 7:127-53) available from the University of Leeds, UK.

Once a compound has been designed or selected by the above methods, the efficiency with which that ligand may bind to or interfere with a HGFA binding site (e.g., an allosteric binding site) may be tested and optimized by computational evaluation. HGFA binding site ligands may interact with the binding site in more than one conformation that is similar in overall binding energy. In those cases, the deformation energy of binding is taken to be the difference between the free energy of the ligand and the average energy of the conformations observed when the ligand binds to the protein.

A ligand designed or selected as binding to or interfering with a HGFA binding site may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available to evaluate compound deformation energy and electrostatic interactions. Examples of programs designed for such uses include: Gaussian 94, revision C (M. J. Frisch, Gaussian, Inc., Pittsburgh, Pa.); AMBER, version 4.1 (P. A. Kollman, University of California at San Francisco,); QUANTA/CHARMM (Molecular Simulations, Inc., San Diego, Calif.); Insight II/Discover (Molecular Simulations, Inc., San Diego, Calif.); DelPhi (Molecular Simulations, Inc., San Diego, Calif.); and AMSOL (Quantum Chemistry Program Exchange, Indiana University). These programs can be implemented, for instance, using a Silicon Graphics workstation, such as an Indigo2 with IMPACT graphics. Other hardware systems and software packages will be known to those skilled in the art.

Another approach encompassed by this disclosure is the computational screening of small molecule databases for ligands or compounds that can bind in whole, or in part, to a HGFA binding site whether in bound or unbound conformation. In this screening, the quality of fit of such ligands to the binding site may be judged either by shape complementarity or by estimated interaction energy (Meng, et al., 1992. J. Comp. Chem., 13:505-24). In addition, these small molecule databases can be screened for the ability to interact with the amino acids in the HGFA binding site as identified herein.

A compound that is identified or designed as a result of any of these methods can be obtained (or synthesized) and tested for its biological activity, for example, binding and/or inhibition of HGFA activity.

Another method involves assessing agents that are antagonists or agonists of the HGFA receptor. A method comprises applying at least a portion of the crystallography coordinates of Tables 7 and/or 8 and/or 9 to a computer algorithm that generates a three-dimensional model of a HGFA:Fab40, HGFA:Fab40.ΔTrp, or HGFA:KQLR:Fab40. ΔTrp complex (“KQLR” disclosed as SEQ ID NO: 21) or the HGFA suitable for designing molecules that are antagonists or agonists and searching a molecular structure database to identify potential antagonists or agonists. In some embodiments, a portion of the structural coordinates of Tables 7 and/or 8 and/or 9 that define a structural feature, for example, all or a portion of a binding site (e.g., an allosteric binding site) for an inhibitor on HGFA. The method may further comprise synthesizing or obtaining the agonist or antagonist and contacting the agonist or antagonist with the HGFA and selecting the antagonist or agonist that modulates the HGFA activity compared to a control without the agonist or antagonists and/or selecting the antagonist or agonist that binds to the HGFA. Activities of HGFA include activation of HGF.

A compound that is identified or designed as a result of any of these methods can be obtained (or synthesized) and tested for its biological activity, for example, binding to HGFA and/or modulation of HGFA activity.

### 7. Machine-Readable Storage Media

Transformation of the structure coordinates for all or a portion of HGFA or the HGFA:Fab40, HGFA: Fab40.ΔTrp, or HGFA:KQLR:Fab40.ΔTrp complex (“KQLR” disclosed as SEQ ID NO: 21), or one of its ligand binding sites, or structurally homologous molecules as defined below, or for the structural equivalents of any of these molecules or molecular complexes as defined above, into three-dimensional graphical representations of the molecule or complex can be conveniently achieved through the use of commercially-available software.

The disclosure thus further provides a machine-readable storage medium including a data storage material encoded with machine-readable data wherein a machine programmed with instructions for using said data displays a graphical three-dimensional representation of any of the molecule or molecular complexes of this disclosure that have been described above. In a preferred embodiment, the machine-readable data storage medium includes a data storage material encoded with machine-readable data wherein a machine programmed with instructions for using the abovementioned data displays a graphical three-dimensional representation of a molecule or molecular complex including all or any parts of an unbound HGFA, a HGFA ligand binding site for an inhibitor or pseudo substrate, or HGFA-like ligand binding site, Fab40, HGFA:Fab40, Fab40. ΔTrp, KQLR (SEQ ID NO: 21), HGFA:Fab40, HGFA: Fab40.ΔTrp, or HGFA:KQLR:Fab40.ΔTrp complex (“KQLR” disclosed as SEQ ID NO: 21) as defined above. In another preferred embodiment, the machine-readable data storage medium includes a data storage material encoded with machine readable data wherein a machine programmed with instructions for using the data displays a graphical three-dimensional representation of a molecule or molecular complex ±a root mean square deviation from the atoms of the amino acids of not more than 0.05 Å.

In an alternative embodiment, the machine-readable data storage medium includes a data storage material encoded with a first set of machine readable data which includes the Fourier transform of structure coordinates, and wherein a machine programmed with instructions for using the data is combined with a second set of machine readable data including the X-ray diffraction pattern of a molecule or molecular complex to determine at least a portion of the structure coordinates corresponding to the second set of machine readable data.

For example, a system for reading a data storage medium may include a computer including a central processing unit (“CPU”), a working memory which may be, for example, RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, track balls, touch pads, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this disclosure may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding site of this disclosure using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this disclosure. Such programs are discussed in reference to the computational methods of drug discovery as described herein. References to components of the hardware system are included as appropriate throughout the following description of the data storage medium.

Machine-readable storage devices useful in the present disclosure include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

### 8. Therapeutic Use

HGFA modulator compounds obtained by methods of the invention are useful in a variety of therapeutic settings. For example, HGFA antagonists designed or identified using the crystal structure of HGFA complex can be used to treat disorders or conditions, where inhibition or prevention of HGFA binding or activity is indicated.

Likewise, HGFA agonists designed or identified using the allosteric binding site and/or crystal structures provided herein can be used to treat disorders or conditions, where induction or stimulation of HGFA is indicated.

An indication can be, for example, inhibition or stimulation of HGF activation and the concomitant activation of a complex set of intracellular pathways that lead to cell growth, differentiation, and migration in a variety of cell types. Another indication can be, for example, in inhibition or stimulation of embryonic development. Still another indication can be, for example, in inhibition or stimulation of tissue regeneration. Yet another indication can be, for example, in inhibition or stimulation of the HGF/Met signaling pathway. Still yet another indication can be, for example, in inhibition or stimulation of invasive tumor growth and metastasis.

HGFA antagonists are also useful as chemosensitizing agents, useful in combination with other chemotherapeutic drugs, in particular, drugs that induce apoptosis. Examples of other chemotherapeutic drugs that can be used in combination with chemosensitizing HGFA inhibitors include topoisomerase I inhibitors (e.g., camptothecin or topotecan), topoisomerase II inhibitors (e.g., daunomycin and etoposide), alkylating agents (e.g., cyclophosphamide, melphalan and BCNU), tubulin-directed agents (e.g., taxol and vinblastine), and biological agents (e.g., antibodies such as anti CD20 antibody, IDEC 8, immunotoxins, and cytokines). Examples of other chemotherapeutic drugs that can be used in combination with chemosensitizing HGF β inhibitors include topoisomerase I inhibitors (e.g., camptothecin or topotecan), topoisomerase II inhibitors (e.g., daunomycin and etoposide), alkylating agents (e.g., cyclophosphamide, melphalan and BCNU), tubulin-directed agents (e.g., taxol and vinblastine), and biological agents (e.g., antibodies such as anti CD20 antibody, IDEC 8, anti-VEGF antibody, immunotoxins, and cytokines). Other examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in the definition of “chemotherapeutic agent” above are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

The following are examples of the methods and compositions of the invention. It is understood that various other embodiments may be practiced, given the general description provided above.

### EXAMPLES

### Materials and Methods

Antibody phage display: Synthetic antibody libraries displayed bivalent Fab fragments on the M13 phage and the diversity was generated by use of oligo-directed mutagenesis in three CDRs of the heavy chain. The details of the Fab libraries were described previously (Lee et al, 2004a; Lee et al, 2004b). Nunc 96-well Maxisorp immunoplates were coated overnight at 4° C. with HGFA (10 μg/ml) and then blocked for 1 hour at room temperature with phage blocking buffer PBST (PBS, 1% (w/v) BSA, 0.05% (v/v) Tween 20). The antibody phage libraries were added to the HGFA-coated plates and incubated overnight at room temperature. The plates were washed with PBT (PBS, 0.05% (v/v) Tween-20) buffer and bound phage were eluted with 50 mM HCl-500 mM NaCl for 30 min and neutralized with an equal volume of 1 M Tris-HCl, pH 7.5. Recovered phage was amplified in E. coli XL-1 blue cells. During subsequent selection rounds, incubation of antibody phage with the antigen-coated plates was reduced to 2-3 hours and the stringency of plate washing was gradually increased.

Affinity maturation of Ab39: Ab39 was affinity-maturated by the use of monovalent Fab instead of bivalent Fab to reduce potential avidity effects during selection. To improve the efficiency of affinity maturation, stop codons were introduced to CDR-L3. Three different CDR combinatorial libraries, L1/L2/L3, L3/H1/H2 and L3/H3 were targeted for randomization using a “soft randomization” strategy that maintains a wild-type sequence bias such that selected positions are mutated only 50% of the time (Liang et al., 2007). For selecting affinity-matured clones, phage libraries were subjected to plate sorting for the first round and followed by four rounds of solution phase sorting as described before (Liang et al., 2007). Increased stringency was used during four rounds of solution phase sorting by decreasing the amount of biotinylated HGFA. Excess amounts of unlabelled HGFA (500˜2000-fold) were added into the last two rounds of selections to compete off the fast off-rate binders. Off-rate selection strategies were used since Ab39 has a relatively high association rate constant (kₒₙ) but a relatively fast dissociation rate constant (k_{off}) (Table 1). The anti-HGFA Fabs were subsequently reformatted to IgGs.

Antibody reformatting and determination of dissociation rate constants to HGFA: Anti-HGFA Fabs were reformatted into human IgG1 by cloning the V_{L} and V_{H} regions of individual clones into LPG3 and LPG4 vector, respectively (Liang et al, 2007). The full-length antibodies were transiently expressed in Chinese Hamster Ovary cells and purified on a protein-A column. To determine binding affinities of the reformatted anti-HGFA antibodies, surface plasmon resonance measurements on a BIAcore™-3000 instrument (GE Health Care, NJ) were performed. Rabbit anti-human IgG were chemically immobilized (amine coupling) on CM5 biosensor chips and the anti-HGFA antibodies were captured to give approximately 250 response units (RU). For kinetics measurements, two-fold serial dilutions of HGFA or active site-blocked HGFA (0.9 nM to 250 nM) were injected in PBT buffer at 25° C. with a flow rate of 30 μl/min. HGFA-KQLR (“KQLR” disclosed as SEQ ID NO: 21) was produced by incubating HGFA with two fold molar excess of Ac-KQLR-cmk (“KQLR” disclosed as SEQ ID NO: 21) for 2 hours at room temperature, followed by size exclusion chromatography to remove non-incorporated Ac-KQLR-cmk (“KQLR” disclosed as SEQ ID NO: 21). Association rates (kₒₙ) and dissociation rates (k_{off}) were obtained by using a simple one-to-one Langmuir binding model (BIAEvaluation) and the equilibrium dissociation constants (K_{D}) were calculated (k_{off}/kₒₙ). Longer injection (5 min) of 2-fold serial dilution of HGFA or HGFA-KQLR (“KQLR” disclosed as SEQ ID NO: 21) (1.5 nM to 3000 nM) over captured antibody (Ab40.ΔTrp) sensor chip was implemented to achieve maximal binding (Rₘₐₓ) and reach the steady state. The values of R_{eq} (20-80% of Rₘₐₓ) were calculated and plotted individually against C (concentration of HGFA or HGFA-KQLR (“KQLR” disclosed as SEQ ID NO: 21)) using BIAEvaluation Software to determine KD at the steady state analysis.

HGFA purification, enzymatic kinetic assays and competition ELISA: HGFA (Val373-Ser655 (shown in Table 5) plus a tag comprising amino acid sequence AAAHHHHHH (SEQ ID NO:13)) was produced by use of a baculovirus—insect cell expression system and purified on a Ni-NTA-agarose column, followed by size exclusion chromatography as described (Kirchhofer et al, 2003). Pro-HGF activation assays with active site-titrated HGFA were carried out essentially as described (Kirchhofer et al., 2003) using serial dilutions of antibody incubated with 1 nM HGFA and 25 μg/ml of ¹²⁵I-pro-HGF. For chromogenic substrate assays with Chromogenix S-2266 (H-D-Valyl-L-leucyl-L-arginine-paranitroanilide) (Diapharma, Westchester, Ohio), 5 nM HGFA was incubated for 40 min in 96-well plates with increasing concentrations of antibodies in TNCT buffer (20 mM Tris, pH 8.0, 150 mM NaCl, 5 mM CaCl₂, 0.01%-Triton X-100). After addition of S-2266 (0.24 mM˜Kₘ) the linear rates of the increase in absorbance at 405 nm were measured on a kinetic microplate reader (Spectramax-M5, Molecular devices, Sunnyvale, Calif.). Enzyme kinetic measurements for Ab39 and Ab40 were carried out with 3 nM HGFA incubated with various antibody concentration (1 μM-0.004 μM in 3-fold dilutions) in TNCT buffer for 40 min. Various concentrations of Chromogenix S-2266 were added and the linear rates of absorbance increase at 405 nm were measured. Eadie-Hofstee plots of the data obtained (v versus v/[S]) were indicative of a competitive inhibition mechanism. Competition ELISA experiments were performed to evaluate effect of Ab40 on KD1 binding to HGFA. 96-well Maxisorp plate coated with HGFA (1 μg/ml) were incubated with increasing concentrations of Ab40 in PBST buffer for 2 hours, followed by addition of 1 nM biotinylated KD1 for 15 minutes. Biotinylated KD1 that was bound to HGFA was detected by streptavidin-HRP conjugates.

Competition HGFA binding ELISA experiments: For binding specificity measurements, 96-well Maxisorp plates were coated with 2 μg/ml of HGFA, matriptase, urokinase-type plasminogen activator or factor XIIa and incubated with 10 μg/ml of anti-HGFA IgG in PBST buffer for at least 1 hour at room temperature. The plates were washed with PBT buffer and bound antibodies were detected with anti-human antibody HRP conjugate diluted 1:2500 in PBST buffer, developed with TMB substrate for approximately 5 minutes, quenched with 1 M H₃PO₄, and absorbance was measured on a microplate reader at 450 nm.

Crystallography: Fab40 and Fab40.ΔTrp was expressed in E. coli and purified by using protein-G sepharose followed by cation exchange chromatography. Fab40 had the following amino acid sequence:

light chain:
(SEQ ID NO: 9)
DIQMTQSPSSLSASVGDRVTITCRASQDVSTAVAWYQQKPGKAPKLLIY
SASFLYSGVPSRFSGSGSGTDFTLTISSLQPEDFATYYCQQSNRAPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
heavy chain:
(SEQ ID NO: 10)
EVQLVESGGGLVQPGGSLRLSCAASGFTINGTYIHWVRQAPGKGLEWVG
GIYPAGGATYYADSVKGRFTISADTSKNTAYLQMNSLRAEDTAVYYCAK
WWAWPAFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTH.

Fab40.ΔTrp had the following amino acid sequence:

light chain:
(SEQ ID NO: 11)
DIQMTQSPSSLSASVGDRVTITCRASQDVSTAVAWYQQKPGICAPKLLI
YSASFLYSGVPSRFSGSGSGTDFTLTISSLQPEDFATYYCQQSNRAPAT
FGQGTKVEIKRTVAAPSVF1FPPSDEQLKSGTASVVCLLNNFYPREAKV
QWKVDNALQSGNSQESVTEQDSKDSTYSLSSTLTLSICADYEKHKVYAC
EVTHQGLSSPVTKSFNRGEC
heavy chain:
(SEQ ID NO: 12)
EVQLVESGGGLVQPGGSLRLSCAASGFTINGTYMWVRQAPGKGLEWVGG
IYPAGGATYYADSVKGRFTISADTSKNTAYLQMNSLRAEDTAVYYCAKW
AWPAFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTH.

Fab40 and Fab.ΔTrp are also described in co-owned co-pending U.S. provisional patent application Ser. No. ______, filed 19 Oct. 2009 (which is hereby incorporated by reference in its entirety). Complexes between (a) HGFA and Fab40 (b) HGFA and Fab40.ΔTrp or (c) HGFA-KQLR (“KQLR” disclosed as SEQ ID NO: 21) and Fab40.ΔTrp were formed by mixing in a 1:2 molar ratio and purified by size exclusion chromatography (Superdex 200). The complexes were concentrated to 10 mg/ml in 10 mM HEPES pH 7.2, 150 mM NaCl. HGFA/Fab40 and HGFA-KQLR/Fab40.ΔTrp complexes (“KQLR” disclosed as SEQ ID NO: 21) yielded crystals under 14% PEG 10,000, 100 mM HEPES pH 7.2, while HGFA/Fab40.ΔTrp yielded crystals under 10% PEG 10,000, 100 mM HEPES pH 7.5. For X-ray data collection, the crystals were transferred to 14% PEG 10,000, 100 mM HEPES pH 7.2, 20% glycerol and immersed in liquid nitrogen. X-ray data were collected at 100 K, either at beam line 9-2 at SSRL/SLAC(HGFA/Fab40) or at ALS beamline 5.0.2 (HGFA/Fab40.ΔTrp and HGFA-KQLR/Fab40.ΔTrp (“KQLR” disclosed as SEQ ID NO: 21)) and reduced using HKL2000 (Otwinowski and Minor, 1997). The structure was solved by molecular replacement using PHASER (McCoy et al, 2005) and refined using CNX (Accelrys) together with elements of the CCP4 suite (CCP4, 1994)(CCP4, 1994). Data reduction and model refinement statistics appear in Table 2. Molecular graphics figures were prepared using PyMOL (DeLano, W. L. The PyMOL Molecular Graphics System, 2002).
Coordinates: Coordinates for the three complexes are as follows: HGFA/Fab40 [Table 7], HGFA/Fab40. ΔTrp [Table 8] and HGFA-KQLR/Fab40. ΔTrp (“KQLR” disclosed as SEQ ID NO: 21) [Table 9].

TABLE 1
Antibody binding to HGFA (“KQLR” disclosed as SEQ ID NO: 21)

| | HGFA | | | HGFA-KQLR complex | | |
|---|---|---|---|---|---|---|
| | kₒₙ | k_{off} | K_{D} | kₒₙ | k_{off} | K_{D} |
| | (×10⁵ M⁻¹ s⁻¹) | (×10⁻⁴ s⁻¹) | (nM) | (×10⁵ M⁻¹ s⁻¹) | (×10⁻⁴ s⁻¹) | (nM) |
| Ab39 | 5.2 ± 0.5 | 53.0 ± 2.0 | 10.3 ± 1.3 | n.d. | n.d. | n.d. |
| Ab40 | 10.8 ± 0.46 | 1.75 ± 0.08 | 0.16 ± 0.01 | 3.2 ± 0.14 | 4.3 ± 0.13 | 1.35 ± 0.1 |
| ^{a}Ab40.ΔT | — | — | 150 ± 9.1 | — | — | 161 ± 7.6 |
| rp | | | | | | |

n.d. not determined
^{a}the K_{D} values were determined using steady state affinity measurements
Generally, throughout the application, IgG form of antibody is designated with prefix Ab and Fab form is designated with prefix Fab. The amino acids of Fab are indicated with a letter-code followed by the residue number, followed by H for heavy chain and L for light chain

TABLE 2
Data Collection and Refinement (“KQLR” disclosed as SEQ ID NO: 21)

| | | HGFA/ | HGFA- |
|---|---|---|---|
| | HGFA/Fab40 | Fab40.ΔTrp | KQLR/Fab40.ΔTrp |
| Data collection | | | |
| Space group | P1 | P2₁ | C222₁ |
| Cell dimensions | | | |
| a, b, c (Å) | a = 38.94, b = 48.93, | a = 72.36, b = 89.53, | a = 80.36, b = 147.89, |
| | c = 96.03 | c = 118.47 | c = 146.37 |
| α, β, γ (°) | α = 98.10, β = 95.01, | β = 91.08 | |
| | γ = 103.89 | | |
| Resolution (Å) | 50-2.35 (2.43-2.35) | 50-2.90 (3.00-2.90) | 50-2.70 (2.8-2.7) |
| Rsym^{a,b} | 0.050 (0.198) | 0.094 (0.505) | 0.143 (0.652) |
| I/σI^{b} | 15 (2.9) | 14.0 (2.7) | 15 (2.9) |
| Completeness (%)^{b} | 94.9 (86.9) | 98.8 (98.3) | 99.4 (97.3) |
| Redundancy | 2.0 (1.9) | 3.7 (3.6) | 7.3 (7.4) |
| Refinement | | | |
| Resolution (Å) | 20-2.35 | 20-2.90 | 20-2.70 |
| No. of reflections | 25746 | 32630 | 23158 |
| Final R^{c}, R_{FREE} | 0.237, 0.291 | 0.216, 0.275 | 0.227, 0.278 |
| No. of atoms | | | |
| Protein | 5046 | 9927 | 5138 |
| Ligand | 28 | 56 | 68 |
| Water | 168 | 153 | 133 |
| B-factors (average) | | | |
| Protein | 65.27 | 68.92 | 35.86 |
| Ligand | 91.72 | 138.26 | 76.06 |
| Water | 58.37 | 39.42 | 29.65 |
| R.m.s. deviations | | | |
| Bond lengths (Å) | 0.008 | 0.008 | 0.007 |
| Bond angles (°) | 1.330 | 1.231 | 1.420 |

^{a}Rsym = Σ | | I | − | <I> | |/Σ | <I>|, where I is the intensity of a single observation and <I> is the average intensity for symmetry equivalent observations.
^{b}In parenthesis, for the highest resolution shell.
^{c}R = Σ | Fo − Fc |/Σ | Fo |, where Fo and Fc are observed and calculated structure factor amplitudes, respectively.

TABLE 3
Contact distances between HGFA and Fab residues
(“KQLR” disclosed as SEQ ID NO: 21)

| | | | Contact distance(Å) | | |
|---|---|---|---|---|---|
| | | | HGFA/ | HGFA/ | HGFA-KQLR |
| # | Fab residue | HGFA residue | Fab40 | Fab40.ΔTrp | Fab40.ΔTrp |
| 1 | H: Tyr 52[OH] | Phe 59 [O] | 3.3 | 3.4 | >3.5 |
| 2 | H: Tyr 52 [OH] | Ser 60b [Oγ] | 2.6 | 3.0 | 2.5 |
| 3 | H: Tyr 58 [OH] | Pro 90 [O] | 2.7 | 2.6 | 2.7 |
| 4 | H: Tyr 33 [OH] | Tyr 91 [O] | 2.6 | >3.5 | >3.5 |
| 5 | H: Trp 95 [Nε1] | Tyr 94 [O] | 3.0 | 2.7 | 2.9 |
| 6 | H: Lys 64 [Nζ] | Asp 240 [Oδ2] | 3.4 | >3.5 | >3.5 |
| 7 | H: Ala 53 [O] | Arg 61 [Nε] | 3.5 | 3.2 | 3.1 |
| 8 | H: Gly 54 [O] | Tyr 88 [N] | 3.1 | 2.9 | 3.0 |
| 9 | H: Gly 55 [O] | Lys 87 [Nζ] | 2.9 | 2.9 | 2.9 |
| 10 | H: Thr 57 [O] | Arg 241 [Nω′] | 2.5 | 2.3 | 2.3 |
| 11 | H: Trp 96 [O] | Ser 95 [Oγ] | 3.0 | >3.5 | >3.5 |
| 12 | H: Trp 96 [O] | Val 96 [N] | 3.2 | >3.5 | >3.5 |
| 13 | H: Gly 31 [O] | Ser 60b [Oγ] | >3.5 | 3.5 | >3.5 |
| 14 | H: Pro 52a [O] | Arg 61 [Nω′] | >3.5 | 3.5 | 3.5 |
| 15 | H: Ala 97 [O] | Ser 95 [Oγ] | >3.5 | >3.5 | 2.6 |
| 16 | H: Ala 97 [O] | Phe 97 [N] | >3.5 | >3.5 | 3.4 |
| 17 | H: Ala 97 [O] | Val 96 [N] | >3.5 | >3.5 | 3.5 |
| 18 | L: Arg 93 [Nω] | Asn 179 [Oδ1] | 2.7 | >3.5 | >3.5 |
| 19 | L: Arg 93 [Nω′] | Asn 179 [Oδ1] | 2.7 | >3.5 | >3.5 |
| 20 | L: Ala 94 [N] | Tyr 91 [OH] | 2.9 | 2.6 | 2.9 |
| 21 | L: Ser 91 [O] | His 101 [Nε2] | 3.0 | 3.0 | 3.0 |
| 22 | L: Asn 92 [Oδ1] | Asn 179 [Nδ2] | 3.0 | 3.1 | 3.2 |
| 23 | L: Arg 93 [Nω] | Asn 233 [O] | >3.5 | >3.5 | 3.5 |
| 24 | L: Arg 93 [Nω′] | Asp 236 [Oδ2] | >3.5 | >3.5 | 3.3 |

TABLE 4
HGFA

MGRWAWVPSP WPPPGLGPFL LLLLLLLLLP RGFQPQPGGN
RTESPEPNAT ATPAIPTILV TSVTSETPAT SAPEAEGPQS
GGLPPPPRAV PSSSSPQAQA LTEDGRPCRF PFRYGGRMLH
ACTSEGSAHR KWCATTHNYD RDRAWGYCVE ATPPPGGPAA
LDPCASGPCL GSCSNTQD PQSYHCSCPR AFTGKDCGTE
KCFDETRYEY LEGGDRWARV RQGHVEQCEC GRTWCEGT
RHTACLSSPC LNGGTCHLIV ATGTTVCACP PGFAGRLCNI
EPDERCFLGN GTGYRGVAST SASGLSCLAW NSDLLYQELH
VDSVGAAALL GLGPHAYCRN PDNDERPWCY VKDSALSWE
YCRLEACESL TRVQLSPDLL ATLPEPASPG RQACGRRHKK
RTFLRPRIIG GSSSLPGSHP WLAAIYIGDS FCAGSLVHTC
WVVSAAHCFS HSPPRDSVSV VLGQHFFNRT TDVTQTFGIE
KYIPYTLYSV FNPSDHDLVL IRLKKKGDRC ATRSQFVQPI
CLPEPGSTFP AGHKCQIAGW GHLDENVSGY SSSLREALVP
LVADHKCSSP EVYGADISPN MLCAGYFDCK SDACQGDSGG
PLACEKNGVA YLYGIISWGD GCGRLHKPGV YTRVANYVDW
INDRIRPPRR LVAPS (SEQ ID NO: 14)

TABLE 5
Activated HG FA

| | |
|---|---|
| 373 | vqlspdll atlpepaspg rqacgrrhkk rtflrpriig gssslpgshp |
| 421 | wlaaiyigds fcagslvhtc wvvsaahcfs hspprdsvsv vlgqhffnrt tdvtqtfgie |
| 481 | kyipytlysv fnpsdhdlvl irlkkkgdrc atrsqfvqpi clpepgstfp aghkcqiagw |
| 541 | ghldenvsgy ssslrealvp lvadhkcssp evygadispn mlcagyfdck sdacqgdsgg |
| 601 | placekngva ylygiiswgd gcgrlhkpgv ytrvanyvdw indrirpprr lvaps |
| | (SEQ ID NO: 15) |

TABLE 6
HGFA serine protease domain

IIGGSSSLPGSHPWLAAIYIGDSFCAGSLVHTCWVVSAAHCFSHSPPRD
SVSVVLGQHFFNRTTDVTQTFGIEKYIPYTLYSVFNPSDHDLVLIRLKK
KGDRCATRSQFVQPICLPEPGSTFPAGHKCQIAGWGHLDENVSGYSSSL
REALVPLVADHKCSSPEVYGADISPNMLCAGYFDCKSDACQGDSGGPLA
CEKNGVAYLYGIISWGDGCGRLHKPGVYTRVANYVDWINDRIR
(SEQ ID NO: 16)

### Results

### Generation of Anti-HGFA Phage Antibody

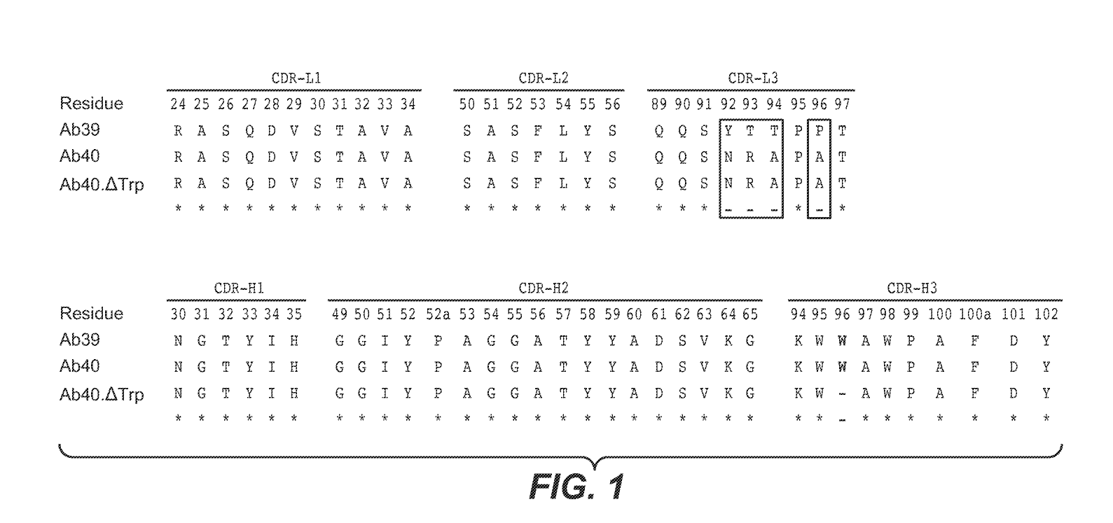

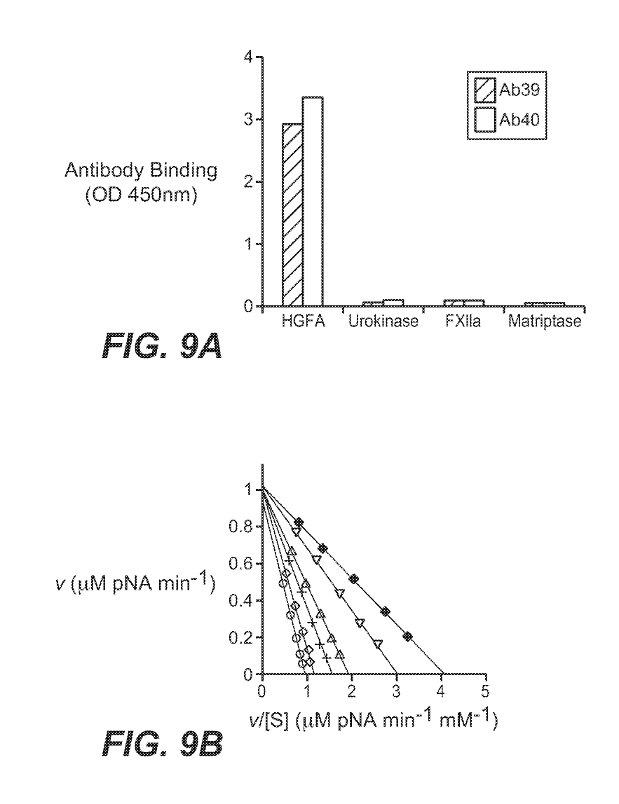

Ab39 was identified by screening of a synthetic F(ab′)₂ phage display library (Wu et al, 2000). Ab39 was subsequently affinity-maturated as described in the methods. The improvement in binding affinity as measured by surface plasmon resonance experiments was 64-fold (Table 1), due to four changes in the sequence of CDR-L3 (FIG. 1). The binding specificities of both Ab39 and Ab40 were assessed by using an ELISA to measure binding to structurally related proteases, including the closest homologues factor XIIa and urokinase (Miyazawa et al., 1993). The results demonstrated a complete lack of binding to factor XIIa, urokinase and matriptase, suggesting good specificity (FIG. 9).

### Enzyme Kinetics and Effect of Active-Site Occupancy by Ab40 Binding

Ab40 inhibited the cleavage of pro-HGF into the α/β-heterodimer mediated by HGFA (FIG. 2a) with a potency that agreed with its binding affinity (Table 1). The inhibitory effects of Ab40 and Ab39 were also assessed in enzymatic assays using the synthetic para-nitroanilide substrate Chromogenix S-2266 (H-D-Val-Leu-Arg-pNA). The enzymatic activity of HGFA was only partially inhibited by Ab40 (FIG. 2b), with a maximum inhibition of about 60% under the chosen experimental conditions. Eadie-Hofstee plots demonstrated that the inhibition mechanism was competitive since Ab40 (and Ab39) increased the Kₘ^{app} but not Vₘₐₓ^{app} values (FIG. 2c, 9b). In accordance with partial inhibition, the slopes (−Kₘ^{app}) approached a finite limit at high Ab40 concentration. Similar results were obtained with the parental Ab39 (data not shown), demonstrating that Ab39 and Ab40 were partial competitive inhibitors of HGFA. To analyze the influence of active site occupancy on the antibody binding, we measured antibody binding to HGFA in the presence of small molecule and macromolecular inhibitors. Benzamidine, which only binds in the S1 pocket of trypsin-like serine proteases, did not interfere with binding of Ab40 to HGFA (data not shown). A peptidic inhibitor matching the pro-HGF cleavage sequence coupled to a war-head group (Ac-KQLR-cmk (“KQLR” disclosed as SEQ ID NO: 21)) was used to covalently modify HGFA in the active site, where it occupied the S4-S1 subsites. Surface plasmon resonance studies with HGFA-KQLR complex (“KQLR” disclosed as SEQ ID NO: 21) showed that the irreversibly bound peptidic inhibitor interfered with the binding of Ab40 (FIG. 3a-b). An 8-fold decrease in affinity of Ab40 binding to HGFA-KQLR complex (“KQLR” disclosed as SEQ ID NO: 21) compared to HGFA was observed (Table 1). The Kunitz domain inhibitor KD1, which interacts with the extended active-site region (Shia et al, 2005), also interfered with Ab40 binding in surface plasmon resonance experiments (FIG. 3c). In agreement, a competition ELISA showed moderate inhibition of KD1 binding to HGFA by Ab40 (FIG. 3d). In summary, binding of Ab40 to HGFA was influenced by inhibitor occupancy at extended subsites, including S2-S4 but not S1.

### Structure of the HGFA/Fab40 Complex Reveals the ‘Allosteric Switch’

The 2.35 Å resolution structure of the HGFA/Fab40 complex shows that Fab40 uses all six CDR loops to bind to a flat epitope at the periphery of the substrate binding cleft, encompassing the 60-loop (e.g., native number 451-454; chymotrypsin numbers 60A-D) and 99-loop (e.g., native numbers 490-493; chymotrypsin numbers 96, 97, 98, and 99a) (FIG. 4a-b). The conformation of the catalytic triad (His-57, Asp-102 and Ser-195) has no significant changes compared to other known structures of HGFA (FIG. 4b) and the substrate subsites S1-S4 are unoccupied (FIG. 5a). The closest atom of Fab40 is >15 Å from the active site Ser195 residue indicating an allosteric mode of inhibition. The key feature of the HGFA/Fab40 complex is a large conformational change in the 99-loop (FIG. 4b and FIG. 10, illustrating the quality of the electron density map). No other significant changes in the HGFA structure are observed suggesting that this is the reason for antibody induced inhibition. The ‘allosteric switch’, embodied in the conformation of the 99-loop, is evident from the comparison of the HGFA/Fab40 structure with other structures of HGFA (Shia et al, 2005; Wu et al, 2007), in which the 99-loop is in a ‘competent’ conformation (catalytically active form of HGFA) state. The Phe97 of the 99-loop in the new conformation (‘non-competent’ conformation, catalytically inactive form of HGFA) is buried in a hydrophobic groove formed at the interface of light chain (Tyr49L, Ser50L and Phe53L) and heavy chain (Trp98H and Pro99H) of Fab40 (FIG. 4c). The epitope is centered on Leu-93 of the protruding 99-loop (FIG. 5a,b), which is sandwiched in a cleft between the CDR loops L3 and H3. The 99-loop and 60-loop are involved in intimate contacts mostly with heavy chain CDR residues. The heavy and light chains contribute 65% and 35% of buried surface area to the complex, respectively (FIG. 5c). The total solvent-accessible surface area of HGFA buried upon Fab40 binding is ˜1030 Å². Altogether, 18 hydrogen bonds and one electrostatic interaction (Asp241-Lys64H) are formed between HGFA and Fab40 (Table 3). Several hydrophobic residues like Trp95H, Trp96H, Trp98H and Tyr33H, Tyr52H, Tyr58H bind into small pockets at the back side of the 60- and 99-loops (FIG. 4c). The Fab40 epitope on HGFA has significant overlap with a region corresponding to exosite II in thrombin, an electropositive region that interacts with thrombin regulators (FIG. 5b). However, unlike exosite II interactions in coagulation proteases which are primarily electrostatic in nature (Bock et al, 2007), the binding of Fab40 to HGFA involves mainly hydrogen bonding and van der Waals interactions.

### Flipping the ‘Allosteric Switch’: Engineering Ab40 to Remove the Allosteric Inhibitory Activity

The CDR-H3 loop of Fab40 contains three tryptophan residues (Trp95H, Trp96H and Trp98H) that form the core of the paratope (FIG. 5c). Trp96H is central to the observed conformational change in 99-loop, by docking its large indole side chain in a deep hydrophobic pocket formed by Ala56, Pro90, Tyr88, Val96, Val104 and Ile106 of HGFA (FIG. 6a). A small shift in the main chain as well as the side chain conformation of Val96 is transmitted through the rest of the 99-loop residues (Val96-Asp100), ultimately leading to >1 Å rmsd shift in C_{α} (99-loop) (FIG. 6c,d). To investigate the role of Trp96H in the conformational change and the associated allosteric inhibitory activity, we deleted this residue (Fab40.ΔTrp) to shorten the CDR-H3 loop (FIG. 1). The complex of HGFA with Fab40.ΔTrp was crystallized as described for the wild-type antibody.

The overall structure of HGFA/Fab40.ΔTrp (2.90 Å) is very similar to that of HGFA/Fab40. The changes are very minimal and confined to residues Ala97H and Trp98H in the CDR-H3 loop (FIG. 6e-f). The side chain of Tyr33H flips around its χ₁ torsion angle and partly fills the deep hydrophobic pocket which was occupied by Trp96H in the HGFA/Fab40 structure (FIG. 6b). The size of this hydrophobic pocket is now reduced due to the movement of residues lining this pocket, principally Ser60, Pro90 and Tyr94 of HGFA. Remarkably the 99-loop reverted to the ‘competent’ state, as observed in other structures of HGFA (FIG. 6e-f). Ab40.ΔTrp was no longer an inhibitor of HGFA as determined by enzymatic assays (FIG. 2b). It was striking that such a subtle change was enough to remove the inhibitory activity while retaining binding, albeit with much reduced affinity (Table 1). Moreover, unlike Ab40, presence of the KQLR (SEQ ID NO: 21) inhibitor in the HGFA active site did not affect Ab40.ΔTrp binding as indicated by the similar K_{D} values for either HGFA or HGFA-KQLR complex (“KQLR” disclosed as SEQ ID NO: 21) (FIG. 3e,f). Thus, the data support our hypothesis that the mechanism of allosteric inhibitory activity by Ab40 is primarily driven by a significant change of the 99-loop conformation.

### Structural Determinants for the Allosteric Mechanism of Inhibition

The 99-loop of HGFA is a critical substrate specificity determinant by contributing to interactions with substrate residues P2 and P4. Therefore, to obtain a detailed understanding on how the Ab40-induced movement of the 99-loop impacted these substrate subsite interactions, we attempted to determine the structure of the HGFA-KQLR complex (“KQLR” disclosed as SEQ ID NO: 21). The KQLR sequence (SEQ ID NO: 21) corresponds to the P4-P1 residues of the natural HGFA substrate pro-HGF. Unfortunately, these attempts were not successful in producing crystals of sufficient diffraction quality despite several attempts to optimize the crystallization conditions. As an alternative approach, we then focused our attention on solving the structure of HGFA-KQLR (“KQLR” disclosed as SEQ ID NO: 21) in complex Fab40.ΔTrp, which readily crystallized.

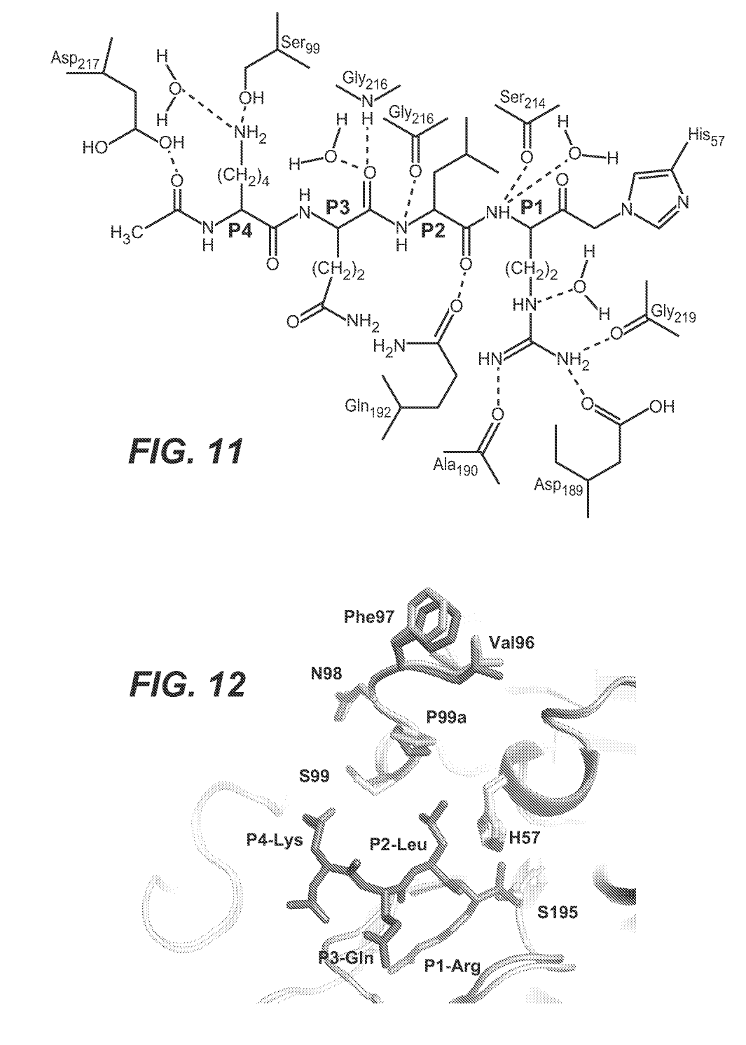

The electron density for peptidic inhibitor, Ac-KQLR-cmk (“KQLR” disclosed as SEQ ID NO: 21) was unambiguous in this 2.70 Å resolution structure. The peptidic inhibitor aligned in the active site groove in a twisted anti-parallel conformation forming the characteristic inter-main chain hydrogen bonds between P1-Arg and Ser214 and between P3-Gln and Gly216 (FIG. 11). The inhibitor was covalently linked to the catalytic Ser195 and His57 and the mode of binding at S4-S1 subsites are very similar to those observed in the complex of KQLR (SEQ ID NO: 21) with hepsin, another S1A protease family member (Herter et al, 2005). A salt bridge interaction pairs the P1-Arg of the peptidic inhibitor with Asp 189 in the S1 subsite. There appears to be a strong preference for a Leucine at the P2 position, because the S2 subsite is a small hydrophobic pocket formed by residues Pro99a, Ser99, Trp215 and His57. The P2-Leu side chain tightly packs against the Pro99a, suggesting that minor changes in the conformation of Pro99a could have a major influence on P2 specificity (FIG. 7a). Thus the specificity at the S2 subsite for HGFA appears to be a distinguishing feature as is the case for many coagulation proteases. Selectivity for the P3 residue is poor in nearly all S1 peptidases as the enzyme-substrate interaction is limited due to solvent exposure of the P3 side chain. The P3-Glu points outward towards the solvent exposed region of the active site. Unlike most S1 peptidases, which possess poor selectivity for a P4 residue, in HGFA a hydrogen bond with Ser99 stabilizes the P4-Lys (FIG. 7a). Additionally, hydrophobic stabilization to the side chain of P4-Lys is offered by Trp215 of HGFA. The carbonyl oxygen from the N-terminal acetyl group is interacting with Asp217 of HGFA through a hydrogen bond.

The structure of the protease domain of HGFA in HGFA-KQLR/Fab40.ΔTrp complex (“KQLR” disclosed as SEQ ID NO: 21) is very similar to HGFA/Fab40.ΔTrp complex with rmsd (for all atoms) of 0.39 Å (FIG. 12). The conformation of the 99-loop in HGFA-KQLR/Fab40.ΔTrp (“KQLR” disclosed as SEQ ID NO: 21) structure is in the ‘competent’ state as found in HGFA/Fab40.ΔTrp and other structures of HGFA. Thus, the HGFA-KQLR/Fab40.ΔTrp (“KQLR” disclosed as SEQ ID NO: 21) is a good alternative in the absence of a HGFA-KQLR (“KQLR” disclosed as SEQ ID NO: 21) structure, to define the substrate binding subsites in HGFA. Superposition of HGFA/Fab40 structure with HGFA-KQLR/Fab40.ΔTrp (“KQLR” disclosed as SEQ ID NO: 21) revealed a plausible cause for the allosteric inhibition. The movement of the 99-loop lead to a partial collapse of the subsite S2 and the reorganization of subsite S4, perturbing the interactions with substrate residues P2 and P4. First, the movement of the 99-loop residues Pro99a and Ser99, which are part of the S2 pocket, positions them too close to the P2-Leu (FIG. 7b), creating a steric clash. Secondly, the repositioned hydroxyl side chain of the S4 residue Ser99 can no longer form the hydrogen bond with P4-Lys (FIG. 7a,b) and is positioned too close to P2-Leu. Furthermore, the movement of the S2 pocket residue Pro99a also plays a key role in defining the allosteric nature of the inhibition of KD1 binding to HGFA by Ab40. Superposition of HGFA/Fab40 and HGFA/KD1 (Shia et al, 2005) complexes showed that there was no overlap between the KD1- and Fab40 epitopes (FIG. 13). A model of HGFA/Fab40/KD1 predicts a possibility of Pro99a to sterically clash with Cys38 and Leu39 of KD1 at the S2 subsite.

Discussion

Allosteric regulation of an enzyme, by definition, involves an altered catalytic activity originating from a remote effector interaction site (Tsai et al, 2009). A variety of effectors including binding of small molecules or macromolecules, phosphorylation, etc., result in a signal, which may result in a signal, which may either activate or inhibit a particular function of the protein (Swain and Gierasch, 2006). Very few such systems are understood beyond knowledge of the effector interaction site and the site of altered activity. The exact route by which amino acids transmit the allosteric effect is, in general, very poorly known. Recent studies have provided new insight into the structural basis of protease inhibition by antibodies that target the enzyme active site (Farady et al, 2008; Wu et al, 2007). In contrast, the exact molecular mechanisms by which allosteric antibodies interfere with enzyme catalysis remain elusive. The findings presented herein, derived from comprehensive structural and kinetic studies, now provide a detailed view of how an allosteric antibody inhibits protease catalysis. Enzyme kinetic analysis demonstrates that the phage display-derived Ab40 is a competitive inhibitor of HGFA. Yet, Ab40 did not inhibit by ‘classical’ steric hindrance, since it bound to an epitope distant from the active site, thus defining a competitive inhibition mechanism that is allosteric in nature. Most importantly, the structure of Fab40/HGFA complex revealed the underlying conformational changes, i.e. the movement of the 99-loop, thereby establishing the structural basis for a functional conduit between epitope and active site.

The observed 99-loop flexibility is unusual in the family of trypsin-like serine proteases, since is not a part of the so-called ‘activation domain’, which comprises several intrinsically mobile surface loops (Huber and Bode, 1978). A known example of conformational flexibility is observed in the serine protease prostasin (Spraggon et al, 1990).

Ab40 binding was not accompanied by any major structural changes other than the 99-loop movement, strongly indicating that this was the cause for enzyme inhibition. To test this hypothesis, we removed one of the key interactions at the Ab40/HGFA interface, i.e. the hydrophobic contact between Trp96H and Val96 of HGFA. The structure of the generated Trp96H-deletion mutant Ab40.ΔTrp in complex with HGFA showed that the 99-loop had flipped back to the functionally ‘competent’ state, consistent with assay results showing that the Ab40.ΔTrp/HGFA complex was enzymatically active. Therefore, the 99-loop movement could be considered as an ‘allosteric switch’ regulating enzyme activity: the ‘allosteric switch’ is turned ON upon Ab40 binding locking the 99-loop in the ‘non-competent’ conformation, whereas antibody removal or binding of the Ab40.ΔTrp mutant turns the ‘allosteric switch’ OFF allowing the 99-loop to adopt the ‘competent’ conformation.

The question arose as to exactly how the ‘non-competent’ 99-loop conformation interferes with the catalytic machinery. That is, which amino acids are changed when Ab40 binds, and why do those changes alter enzyme activity? The 99-loop does not contribute to the formation of the S1 specificity pocket and binding experiments confirmed that S1-P1 interactions were not affected by Ab40 binding. However, the ‘front’ side of the 99-loop in respect to the Ab40 epitope participates in shaping important substrate subsites and this is the region where obstructions likely arose. The structure of HGFA with the irreversibly bound KQLR peptide (SEQ ID NO: 21) provided a plausible answer. The KQLR peptide (SEQ ID NO: 21) constitutes the P4-P1 sequence of the natural substrate pro-HGF and also contains the P2-P1 residues, i.e. LR, of the synthetic pNA substrate S-2266 used in our enzyme assays.

Structural analysis showed that the ‘non-competent’ conformation of the 99-loop obstructed substrate access to S2 and S4 subsites, due to a steric clash between the P2-Leu and the S2 subsite (Pro99a and Ser99) and the loss of stabilizing interactions between P4-Lys and the S4 subsite. The hydrozyl side chain of Ser99 residue was found to adopt two different conformations, thus acting as a key specificity determinant at the S2 subsite. This observation is analogous to the conformational changes observed in Tyr99 of coagulation factor IX1 (Hopfner et al, 1999). In the ‘competent’ conformation the hydrophobic S2 pocket is ideally shaped to recognize Leu as a P2 residue, consistent with the presence of P2-Leu in the natural substrates pro-HGF and pro-MSP, as well as the synthetic S-2266 substrate. Therefore, the partial collapse of the S2 subsite by Ab40 binding may have sufficed to cause inhibition of enzyme catalysis towards both macromolecular and synthetic substrates. A caveat associated with this structural interpretation is our use of the HGFA-KQLR/Fab40. ΔTrp (“KQLR” disclosed as SEQ ID NO: 21) structure as a surrogate for that of HGFA-KQLR (“KQLR” disclosed as SEQ ID NO: 21), which we failed to crystallize. However, the 99-loop in the HGFA-KQLR/Fab40. ΔTrp (“KQLR” disclosed as SEQ ID NO: 21) structure adopts the ‘competent’ conformation and, therefore, we take this structure as to provide a very good approximation of the S4-S1 interactions with substrate. This view is supported by the observation that the conformation of the KQLR peptide (SEQ ID NO: 21) is virtually identical with that in the related KQLR-hepsin complex (“KQLR” disclosed as SEQ ID NO: 21) (Herter et al 2005). We thoroughly evaluated the influence of inter-molecular contacts on the interpretation of our results. The catalytic triad (H57-D102-S195) is not involved in crystal contacts in any of the three structures. Additionally, the 99-loop is not involved in crystal contacts in the structure of HGFA/Fab40 or HGFA-KQLR/Fab40. ΔTrp (“KQLR” disclosed as SEQ ID NO: 21). However, the 99-loop is stabilized by a symmetry related molecule in the case of HGFA/Fab40. ΔTrp (both molecules in the asymmetric unit). Since the conformation of the 99-loop in HGFA-KQLR/Fab40. ΔTrp (“KQLR” disclosed as SEQ ID NO: 21) is similar as in HGFA/Fab40. ΔTrp, we considered the impact from crystal contacts negligible.

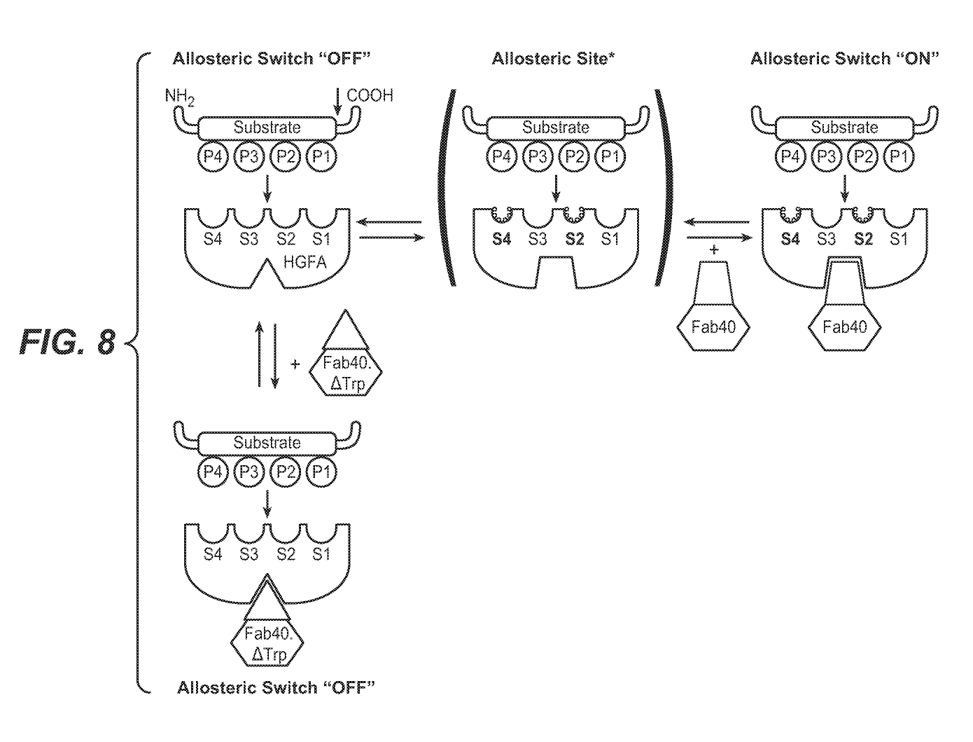

The switch of the 99-loop can be considered as a mobile conduit that connects the inhibitor (i.e. Ab40) binding site with the substrate binding site. Such a view would also provide a suitable framework for understanding the competitive inhibition mode determined in enzymatic assays. Both inhibitor and substrate can apply forces on the 99-loop, albeit from opposite directions, resulting in 99-loop conformations that are sub-optimal with either substrate binding (‘non-competent’ state) or inhibitor binding (‘competent’ state). Structural models indicate that steric clashes occur in both situations, i.e. between the 99-loop and P2-Leu of substrate in the ‘non-competent’ state (FIG. 7c) and between the 99-loop and Trp96H, Ala97H and Trp98H in CDR-H3 of Fab40 in the ‘competent’ state (FIG. 14). Based on classical enzyme kinetics several models of competitive inhibition have been proposed, among them allosteric models, as illustrated by Segel (Segel, 1993). The elucidated allosteric mechanism is a refinement of the model-5 by Segel, as it provides the structural basis of the molecular linkage between inhibitor binding site and active site. The model in FIG. 8 shows the catalytically competent and inhibited states of enzyme in an equilibrium favoring the competent state. In this model, the binding of Ab40 to the transient ‘non-competent’ state (allosteric site*) simply shifts the equilibrium away from the functionally active state, thus driving the major population of enzyme molecules from the ‘allosteric switch’ “OFF” state to the “ON” state. The model also accounts for the competitive nature of HGFA inhibition, in that an increase of substrate concentration will shift the equilibrium to the left, i.e. to the ‘competent’ state of HGFA allowing catalysis to proceed. The mutated Ab40.ΔTrp does not impede catalysis, because it only binds to the ‘competent’ state in which the ‘allosteric switch’ is turned OFF. This interpretation is consistent with the generally accepted view of allostery in that effector binding leads to a shift in the ensemble of protein conformations, thus altering the relative populations of particular states. Ab40 binding to HGFA effectively resulted in a shift/redistribution from the ‘competent’ to a ‘non-competent’ state and thus to a functionally impaired enzyme. Extending this view to the Ab40. ΔTrp, it can also be regarded as allosteric effector, which imposes only small or negligible effects on the binding site, thereby sampling the ‘competent’ enzyme conformation.

The ‘allosteric switch’ is a relatively simple allosteric mechanism. It involves only one mobile surface loop, which directly links allosteric effector binding site with the active site. It contrasts with other more complex and less understood allosteric mechanisms, such as cofactor-induced enzyme activation (Olsen and Persson, 2008) or PDZ-domain mediated inhibition/activation of HtrA 1 family members (Sohn et al, 2007), where effector binding is associated with multiple, short- and long-range conformational changes. Nevertheless, despite its relative simplicity it may replicate a naturally occurring allosteric regulation mechanism of HGFA activity by yet unknown effector molecules. In particular, the Ab40 binding site significantly overlaps with the exosite II of thrombin and the corresponding region of coagulation factors IX and X, which are docking sites for various allosteric effectors, including heparin. However, the corresponding region of HGFA appears ill-suited to bind heparin, because the prominent cluster of Arg and Lys residues that mediate exosite-heparin interactions in coagulation factors is minimally represented in HGFA.

Research on allosteric inhibitors have been actively pursued for kinases (Vajpai et al., 2008) and GPCR's (Raddatz et al., 2007) among others. It is interesting to note that the mechanism of allosteric inhibition by Ab40 is similar to some of the other known allosteric small molecule inhibitors, in either case, the allosteric inhibitor act by restricting the conformational flexibility in the enzyme active site (Goodey and Benkovic, 2008; Lee and Craik, 2009).

Allosteric anti-protease antibodies may have great therapeutic potential, since they are potent and highly specific and are safeguarded from any inadvertent processing by their target protease. However, their use as therapeutic agents is currently limited to extracellular proteases, while intracellular proteases are primarily targeted by orthosteric small molecule inhibitors. In this respect, our findings may suggest new approaches to identify allosteric ‘hot spots’ that might be amenable to structure-based design of allosterically acting peptidic or small molecule inhibitor (Hardy and Wells, 2004). Specifically, the herein described interaction of Trp96H with a large hydrophobic pocket (‘hot spot’) is critical in stabilizing the non-competent 99-loop conformation, yet the existence of this pocket could not have been predicted from other HGFA structures. Thus, large scale screening of Fab phage display libraries in conjunction with Fab/protease structure determination may identify promising allosteric ‘hot spots’. Such an approach should further benefit from the intrinsic property of Fabs to facilitate crystallization of proteins (Tereshko et al, 2008).

Another aspect of our study is the potential usefulness of the anti-HGFA antibody to experimentally address the roles of HGFA in pathologic pathways. For instance, it was suggested that the ability of HGFA to efficiently process pro-HGF and consequently stimulate the HGF/Met signaling pathway may contribute to cancer growth (Kataoka et al., 2003a). Ab40 binds to and blocks mouse HGFA equally well as human HGFA (data not shown), making it an ideal reagent for further investigation of HGFA function in mouse tumor models.

### PARTIAL REFERENCE LIST

Adams, G. P., and Weiner, L. M. (2005). Monoclonal antibody therapy of cancer. Nat Biotech 23, 1147-1157.
Barrett, A. J., Rawlings, N. D., and Woessner, J. F. (1998). Handbook of Proteolytic Enzymes. (San Diego: Academic Press).
Bjelke, J. R., Olsen, O. H., Fodje, M., Svensson, L. A., Bang, S., Bolt, G., Kragelund, B. B., and Persson, E. (2008). Mechanism of the Ca2+-induced Enhancement of the Intrinsic Factor VIIa Activity. J Biol Chem 283, 25863-25870.
Bock, P. E., Panizzi, P., and Verhamme, I. M. (2007). Exosites in the substrate specificity of blood coagulation reactions. J Thromb Haemost 5 Suppl 1, 81-94.
CCP4 (1994). The CCP4 suite: Programs for protein crystallography. Acta Crystallogr D50, 760-763.
Changeux, J. P., and Edelstein, S. J. (2005). Allosteric mechanisms of signal transduction. Science 308, 1424-1428.
del Sol, A., Tsai, C. J., Ma, B., and Nussinov, R. (2009). The origin of allosteric functional modulation: multiple pre-existing pathways. Structure 17, 1042-1050.
Di Cera, E. (2006). A structural perspective on enzymes activated by monovalent cations. J Biol Chem 281, 1305-1308.
Egeblad, M., and Werb, Z. (2002). New functions for the matrix metalloproteinases in cancer progression. Nature Rev. Cancer 2, 161-174.
Eigenbrot, C., and Kirchhofer, D. (2002). New Insight into How Tissue Factor Allosterically Regulates Factor VIIa. Trends in Cardiovascular Medicine 12, 19-26.
Farady, C. J., Egea, P. F., Schneider, E. L., Darragh, M. R., and Craik, C. S. (2008). Structure of an Fab-Protease Complex Reveals a Highly Specific Non-canonical Mechanism of Inhibition. Journal of Molecular Biology 380, 351-360.
Fenton, A. W. (2008). Allostery: an illustrated definition for the ‘second secret of life’. Trends in biochemical sciences 33, 420-425.
Friedrich, R., Panizzi, P., Fuentes-Prior, P., Richter, K., Verhamme, I., Anderson, P. J., Kawabata, S.-I., Huber, R., Bode, W., and Bock, P. E. (2003). Staphylocoagulase is a prototype for the mechanism of cofactor-induced zymogen activation. Nature 425, 535-539.
Goodey, N. M., and Benkovic, S. J. (2008). Allosteric regulation and catalysis emerge via a common route. Nature chemical biology 4, 474-482.
Gunasekaran, K., Ma, B., and Nussinov, R. (2004). Is allostery an intrinsic property of all dynamic proteins? Proteins 57, 433-443.
Hardy, J. A., Lam, J., Nguyen, J. T., O'Brien, T., and Wells, J. A. (2004). Discovery of an allosteric site in the caspases. Proceedings of the National Academy of Sciences of the United States of America 101, 12461-12466.
Hardy, J. A., and Wells, J. A. (2004). Searching for new allosteric sites in enzymes. Current opinion in structural biology 14, 706-715.
Hardy, J. A., and Wells, J. A. (2009). Dissecting an allosteric switch in caspase-7 using chemical and mutational probes. J Biol. Chem.
Hauske, P., Ottmann, C., Meltzer, M., Ehrmann, M., and Kaiser, M. (2008). Allosteric regulation of proteases. Chembiochem 9, 2920-2928.
Hedstrom, L. (2002). Serine protease mechanism and specificity. Chemical reviews 102, 4501-4524.
Herter, S., Piper, D. E., Aaron, W., Gabriele, T., Cutler, G., Cao, P., Bhatt, A. S., Choe, Y., Craik, C. S., Walker, N., et al. (2005). Hepatocyte growth factor is a preferred in vitro substrate for human hepsin, a membrane-anchored serine protease implicated in prostate and ovarian cancers. Biochem J 390, 125-136.
Hooper, N. M. (2002). Proteases in Biology and Medicine. In Essays in Biochemistry (London: Portland Press).
Hopfner, K. P., Lang, A., Karcher, A., Sichler, K., Kopetzki, E., Brandstetter, H., Huber, R., Bode, W., and Engh, R. A. (1999). Coagulation factor IXa: the relaxed conformation of Tyr99 blocks substrate binding. Structure 7, 989-996.
Huber, R., and Bode, W. (1978). Structural basis of the activation and action of trypsin. Acc. Chem. Res. 11, 114-122.
Huntington, J. A. (2008). How Na+ activates thrombin—a review of the functional and structural data. Biological chemistry 389, 1025-1035.
Kabat, E. A., Wu, T. T., Perry, H. M., Gottesman, K. S., and Foeller, C. (1991). Sequences of proteins of immunological interest, fifth edition. (National Institutes of Health, Bethesda Md.).
Kataoka, H., Miyata, S., Uchinokura, S., and Itoh, H. (2003a). Roles of hepatocyte growth factor (HGF) activator and HGF activator inhibitor in the pericellular activation of HGF/scatter factor. Cancer and Metastasis Reviews 22, 223-236.
Kataoka, H., Miyata, S., Uchinokura, S., and Itoh, H. (2003b). Roles of hepatocyte growth factor (HGF) activator and HGF activator inhibitor in the pericellular activation of HGF/scatter factor. Cancer metastasis reviews 22, 223-236.
Kawaguchi, M., Orikawa, H., Baba, T., Fukushima, T., and Kataoka, H. (2009). Hepatocyte growth factor activator is a serum activator of single-chain precursor macrophage-stimulating protein. The FEBS journal 276, 3481-3490.
Kirchhofer, D., Peek, M., L1, W., Stamos, J., Eigenbrot, C., Kadkhodayan, S., Elliott, J. M., Corpuz, R. T., Lazarus, R. A., and Moran, P. (2003). Tissue expression, protease specificity, and Kunitz domain functions of hepatocyte growth factor activator inhibitor-1B (HAI-1B), a new splice variant of HAI-1. J Biol Chem 278, 36341-36349.
Krauss, G. (2003). Biochemistry of Signal Transduction and Regulation. G. Krauss, ed. (New York: Wiley and Sons), pp. 89-114.
Lee, C. V., Liang, W. C., Dennis, M. S., Eigenbrot, C., Sidhu, S. S., and Fuh, G. (2004a). High-affinity human antibodies from phage-displayed synthetic Fab libraries with a single framework scaffold. J Mol Biol 340, 1073-1093.
Lee, C. V., Sidhu, S. S., and Fuh, G. (2004b). Bivalent antibody phage display mimics natural immunoglobulin. J Immunol Methods 284, 119-132.
Lee, G. M., and Craik, C. S. (2009). Trapping moving targets with small molecules. Science 324, 213-215.
Liang, W. C., Dennis, M. S., Stawicki, S., Chanthery, Y., Pan, Q., Chen, Y., Eigenbrot, C., Yin, J., Koch, A. W., Wu, X., et al. (2007). Function blocking antibodies to neuropilin-1 generated from a designed human synthetic antibody phage library. J Mol Biol 366, 815-829.
Luttun, A., Dewerchin, M., Collen, D., and Carmeliet, P. (2000). The role of proteinases in angiogenesis, heart development, restenosis, atherosclerosis, myocardial ischemia, and stroke: insights from genetic studies. Curr. Atheroscler. Rep. 2, 407-416.
McCoy, A. J., Grosse-Kunstleve, R. W., Storoni, L. C., and Read, R. J. (2005). Likelihood-enhanced fast translation functions. Acta Crystallogr D Biol Crystallogr 61, 458-464.
Miyazawa, K., Shimomura, T., Kitamura, A., Kondo, J., Morimoto, Y., and Kitamura, N. (1993). Molecular cloning and sequence analysis of the cDNA for a human serine protease responsible for activation of hepatocyte growth factor. Structural similarity of the protease precursor to blood coagulation factor XII. J Biol Chem 268, 10024-10028.
Monod, J. (1977). Chance and Necessity: Essay on the Natural Philosophy of Modern Biology (Penguin Books).
Olsen, O., and Persson, E. (2008). Cofactor-induced and mutational activity enhancement of coagulation factor VIIa. Cellular and Molecular Life Sciences (CMLS) 65, 953-963.
Otwinowski, Z., and Minor, W. (1997). Processing of X-ray diffraction data collected in oscillation mode. In: Macromolecular Crystallography Part A. Methods in Enzymol. 276, 307-326.
Parr, C., and Jiang, W. G. (2001). Expression of hepatocyte growth factor/scatter factor, its activator, inhibitors and the c-Met receptor in human cancer cells. International journal of oncology 19, 857-863.
Pellicena, P., and Kuriyan, J. (2006). Protein-protein interactions in the allosteric regulation of protein kinases. Current opinion in structural biology 16, 702-709.
Perutz, M. F. (1970). Stereochemistry of cooperative effects in haemoglobin. Nature 228, 726-739.
Peterson, J. R., and Golemis, E. A. (2004). Autoinhibited proteins as promising drug targets. J Cell Biochem 93, 68-73.
Raddatz, R., Schaffhauser, H., and Marino, M. J. (2007). Allosteric approaches to the targeting of G-protein-coupled receptors for novel drug discovery: a critical assessment. Biochemical pharmacology 74, 383-391.
Rawlings, N. D., Morton, F. R., Kok, C. Y., Kong, J., and Barrett, A. J. (2008). MEROPS: the peptidase database. Nucleic Acids Res 36, D320-325.
Segel, I. (1993). Enzyme Kinetics: Behavior and Analysis of Rapid Equilibrium and Steady-State Enzyme Systems, Vol pp 161-226 (New York: Wiley).
Shia, S., Stamos, J., Kirchhofer, D., Fan, B., Wu, J., Corpuz, R. T., Santell, L., Lazarus, R. A., and Eigenbrot, C. (2005). Conformational lability in serine protease active sites: structures of hepatocyte growth factor activator (HGFA) alone and with the inhibitory domain from HGFA inhibitor-1B. J Mol Biol 346, 1335-1349.
Shimomura, T., Miyazawa, K., Komiyama, Y., Hiraoka, H., Naka, D., Morimoto, Y., and Kitamura, N. (1995). Activation of hepatocyte growth factor by two homologous proteases, blood-coagulation factor XIIa and hepatocyte growth factor activator. Eur J Biochem 229, 257-261.
Sohn, J., Grant, R. A., and Sauer, R. T. (2007). Allosteric Activation of DegS, a Stress Sensor PDZ Protease. Cell 131, 572-583.
Spraggon, G., Hornsby, M., Shipway, A., Tully, D. C., Bursulaya, B., Danahay, H., Harris, J. L., and Lesley, S. A. (2009). Active site conformational changes of prostasin provide a new mechanism of protease regulation by divalent cations. Protein Sci 18, 1081-1094.
Swain, J. F., and Gierasch, L. M. (2006). The changing landscape of protein allostery. Current opinion in structural biology 16, 102-108.
Tereshko, V., Uysal, S., Koide, A., Margalef, K., Koide, S., and Kossiakoff, A. A. (2008). Toward chaperone-assisted crystallography: protein engineering enhancement of crystal packing and X-ray phasing capabilities of a camelid single-domain antibody (VHH) scaffold. Protein Sci 17, 1175-1187.
Tsai, C. J., Del Sol, A., and Nussinov, R. (2009). Protein allostery, signal transmission and dynamics: a classification scheme of allosteric mechanisms. Molecular bioSystems 5, 207-216.
Turk, B. (2006). Targeting proteases: successes, failures and future prospects. Nature reviews 5, 785-799.
Vajpai, N., Strauss, A., Fendrich, G., Cowan-Jacob, S. W., Manley, P. W., Grzesiek, S., and Jahnke, W. (2008). Solution conformations and dynamics of ABL kinase-inhibitor complexes determined by NMR substantiate the different binding modes of imatinib/nilotinib and dasatinib. J Biol Chem 283, 18292-18302.
Wells, C. M., and Di Cera, E. (1992). Thrombin is a Na(+)-activated enzyme. Biochemistry 31, 11721-11730.
Wu, Y., Eigenbrot, C., Liang, W.-C., Stawicki, S., Shia, S., Fan, B., Ganesan, R., Lipari, M. T., and Kirchhofer, D. (2007). Structural insight into distinct mechanisms of protease inhibition by antibodies. Proceedings of the National Academy of Sciences 104, 19784-19789.
Xu, Z., Horwich, A. L., and Sigler, P. B. (1997). The crystal structure of the asymmetric GroEL-GroES-(ADP)₇ chaperonin complex. Nature 388, 741-750.
Yu, E. W., and Koshland, D. E., Jr. (2001). Propagating conformational changes over long (and short) distances in proteins. Proceedings of the National Academy of Sciences of the United States of America 98, 9517-9520.

Table 7

Coordinates for HGFA complexed with an anti-HGFA antibody. Table 7 is submitted on a compact disc filed herewith, the contents of which are incorporated by record in its entirety.

Table 8

Coordinates for HGFA complexed with a Fab40. ΔTrp anti-HGFA antibody. Table 8 is submitted on a compact disc filed herewith, the contents of which are incorporated by record in its entirety.

Table 9

Coordinates for HGFA complexed with KQLR (SEQ ID NO: 21) and a Fab40. ΔTrp anti-HGFA antibody.

Table 9 is submitted on a compact disc filed herewith, the contents of which are incorporated by record in its entirety.

Although the foregoing invention has been described in some detail by way of illustration and example for purposes of clarity of understanding, the descriptions and examples should not be construed as limiting the scope of the invention.

What is claimed is:

1. A method of screening for a candidate inhibitor substance that allosterically inhibits HGFA activation of HGFA substrate, said method comprising: detecting binding, if any, of the candidate substance to a HGFA allosteric binding site, wherein the candidate substance is identified by a method comprising comparing amount of HGFA substrate activation in a sample with amount of HGFA substrate activation in a reference sample comprising similar amounts of HGFA and HGFA substrate as the first sample but that has not been contacted with said candidate substance, whereby a decrease in amount of HGFA substrate activation in the first sample compared to the reference sample indicates that the candidate substance is capable of inhibiting HGFA activation of HGFA substrate, wherein the sample comprises HGFA, the candidate substance and the HGFA substrate.

2. A method of screening for a candidate inhibitory substance that allosterically inhibits HGFA activation, the method comprising screening for a candidate inhibitory substance that binds HGFA allosteric binding site and inhibits HGFA activity.

3. The method of claim 2, wherein the method comprising selecting for a substance that binds to at least one, two, three, four, or any number up to all of residues 449, 450, 452, 453, 455, 480, 481, 482, 483, 484, 485, 486, 487, 488, 489, 490, 491, 496, 578, 579, 580, 636, 637, 640, 643, 644 of HGFA.

4. The method of claim 2, wherein the method comprises selecting for a substance that binds to at least one, two, three, four, or any number up to all of residues 449, 450, 452, 482, 484, 485, 486, 487, 488, 489, 490, 491 of HGFA.

5. The method of claim 2, wherein the method comprises selecting for a substance that screening for a substance that binds HGFA in the absence of a compound that blocks HGFA active (catalytic) site, but does not bind HGFA in the presence of the compound that blocks HGFA active site.

6. The method of any of claims 1-4, wherein HGFA in the sample is in an effective amount for activating said HGFA substrate.

7. The method of any of claims 1-6, wherein the HGFA substrate is a polypeptide comprising HGF or fragment thereof comprising a wild type form of the R494-V495 peptide linkage.

8. The method of claim 7, wherein the pro-HGF substrate comprises a cleavage site of human HGF that fits the consensus cleavage site of proteases wherein the cleavage site comprises basic residue at position P1 and two hydrophobic amino acid residues in positions P1′ and P2′.

9. An antagonist molecule that allosterically inhibits HGFA.

10. The antagonist molecule of claim 9, wherein the molecule comprises an antibody or fragment thereof.

11. The antagonist molecule of claim 9, wherein the molecule comprises an anti-HGFA antibody comprising the light chain and heavy chain variable regions of Mab40.

12. A crystal of HGFA complexed with an anti-HGFA antibody comprising a human HGFA comprising an amino acid sequence shown in Table 5 or conservative substitutions thereof complexed with an anti-HGFA antibody comprising (a) a light chain variable domain comprising the amino acid sequence of SEQ ID NO: 9 or conservative substitutions thereof, and (b) a heavy chain variable domain comprising the amino acid sequence of SEQ ID NO: 10 or conservative substitutions thereof.

13. Crystalline form of a complex of HGFA and an anti-HGFA antibody.

14. A crystal of a 1:1 complex of HGFA and an anti-HGFA antibody having a space group having a space group symmetry of P₁ and comprising a unit cell having the dimensions of a, b, and c, wherein a=38.94 Å, b=48.93 Å and c=96.03 Å and α=98.10 Å β=95.01 Å and γ=103.89 Å.

15. A cocrystal of HGFA with an anti-HGFA antibody having the three-dimensional coordinates of Table 7.

16. The crystal of claim 12, wherein the crystal diffracts X-rays for the determination of atomic coordinates to a resolution of 5 Å or better.

17. A composition comprising a crystal of any of claims 12-16, and a carrier.

18. A molecule or molecular complex comprising at least a portion of the allosteric binding site of HGFA comprising an amino acid sequence shown in Table 5 or conservative substitution thereof, wherein the binding site comprises at least one amino acid residue corresponding to residues 446, 449, 450, 452, 453, 455, 480, 481, 482, 483, 484, 485, 486, 487, 488, 489, 490, 491, 496, 499, 501, 578, 579, 580, 636, 637, 640, 643, 644 or mixtures thereof, the binding site defined by a set of points having a root mean square deviation of less than about 0.70 Å from points representing the backbone atoms of the amino acids as represented by the structure coordinates listed in Table 7.

19. A computer-implemented method for causing a display of a graphical three-dimensional representation of the structure of a portion of a crystal of HGFA complexed with an anti-HGFA antibody, or structural homologs thereof, wherein the method comprises:
causing said display of said graphical three-dimensional representation by a computer system programmed with instructions for transforming structure coordinates into said graphical three-dimensional representation of said structure and for displaying said graphical three-dimensional representation,
wherein said graphical three-dimensional representation is generated by transforming said structure coordinates into said graphical three-dimensional representation of said structure,
wherein said structure coordinates comprise structure coordinates of the backbone atoms of the portion of the crystal,
wherein the portion of the crystal comprises an HGFA allosteric binding site,
wherein the crystal has the space group symmetry P₁.

20. The computer-implemented method of claim 19, wherein the HGFA:anti-HGFA antibody crystal comprises a polypeptide comprising an amino acid sequence shown in Table 5 or conservative substitution thereof, and further comprises an antibody comprising (a) a light chain variable domain comprising the amino acid sequence of SEQ ID NO: 9 or conservative substitutions thereof, and (b) a heavy chain variable domain comprising the amino acid sequence of SEQ ID NO: 10 or conservative substitutions thereof.

21. The computer-implemented method of claim 19, wherein the structure coordinates are defined in Table 7.

22. The computer-implemented method of claim 19, wherein the structure coordinates comprise the structure coordinates of the backbone atoms of the amino acid residues corresponding to positions 449, 450, 452, 482, 484, 485, 486, 487, 488, 489, 490, 491 of the sequence shown in Table 4.

23. The computer-implemented method of claim 19, wherein the structure coordinates comprise the structure coordinates of the backbone atoms of the amino acid residues corresponding to positions 446, 449, 450, 452, 453, 455, 480, 481, 482, 483, 484, 485, 486, 487, 488, 489, 490, 491, 496, 499, 501, 578, 579, 580, 636, 637, 640, 643, 644 of the sequence shown in Table 4.

24. The computer-implemented method of claim 19, wherein the structure coordinates are determined by homology modeling.

25. A machine-readable data storage medium comprising a data storage material encoded with machine-readable instructions for:
(a) transforming data into a graphical three-dimensional representation for the structure of a portion of a crystal of HGFA complexed with an anti-HGFA antibody, or structural homologs thereof; and
(b) causing the display of said graphical three-dimensional representation;
wherein said data comprise structure coordinates of the backbone atoms of the amino acids defining an HGFA allosteric binding site; and wherein the crystal or structural homolog has the space group symmetry P₁.

26. A computer system for displaying a three-dimensional graphical representation for the structure of a portion of a crystal of HGFA complexed with an anti-HGFA antibody, or structural homologs thereof, comprising:
(a) a machine-readable data storage medium comprising a data storage material encoded with machine-readable data, wherein said data comprise structure coordinates of the backbone atoms of the amino acids defining an HGFA allosteric binding site, wherein the crystal or structural homolog has the space group symmetry P₁.;
(b) a working memory;
(c) a central processing unit coupled to said working memory and to said machine-readable data storage medium for processing said machine-readable data into sad three-dimensional graphical representation; and
(d) a display coupled to said central processing unit for displaying said three-dimensional graphical representation.

27. A method for obtaining structural information about a molecule or molecular complex comprising applying at least a portion of the structure coordinates of a HGFA complexed with an anti-HGFA antibody to an X-ray diffraction pattern of the molecule or molecular complex's crystal structure to cause the generation of a three-dimensional electron density map of at least a portion of the molecule or molecular complex;
wherein the HGFA:anti-HGFA antibody crystal comprises a polypeptide comprising an amino acid sequence shown in Table 5 or conservative substitution thereof, and further comprises an antibody comprising (a) a light chain variable domain comprising the amino acid sequence of SEQ ID NO: 9 or conservative substitutions thereof, and (b) a heavy chain variable domain comprising the amino acid sequence of SEQ ID NO: 10 or conservative substitutions thereof.;
wherein the HGFA:anti-HGFA antibody crystal diffracts x-rays for the determination of atomic coordinates to a resolution of 5 Å or better.

28. A method of screening for molecules that may be antagonists or agonists of HGFA comprising:
(a) computationally screening agents against a three-dimensional model to identify potential antagonists or agonists of HGFA;
wherein the three-dimensional model comprises a three-dimensional model of at least a portion of a crystal of a HGFA complexed with an anti-HGFA antibody;
wherein the three dimensional model is generated from at least a portion of the structure coordinates of the crystal by a computer algorithm for generating a three-dimensional model of the crystal useful for identifying agents that are potential antagonists or agonists of HGFA;
wherein the HGFA: anti-HGFA antibody crystal comprises a polypeptide comprising an amino acid sequence shown in Table 5 or conservative substitution thereof, and further comprises an antibody comprising (a) a light chain variable domain comprising the amino acid sequence of SEQ ID NO: 9 or conservative substitutions thereof, and (b) a heavy chain variable domain comprising the amino acid sequence of SEQ ID NO: 10 or conservative substitutions thereof; and
wherein the HGFA:anti-HGFA antibody crystal diffracts x-rays for the determination of atomic coordinates to a resolution of 5 Å or better.

29. An HGFA inhibitor comprising sequence Arg-Gln-Leu-Arg (SEQ ID NO: 22).

30. An HGFA inhibitor consisting essentially of sequence Arg-Gln-Leu-Arg (SEQ ID NO: 22).

31. An HGFA inhibitor consisting of sequence Arg-Gln-Leu-Arg (SEQ ID NO: 22).
